(19) United States
(12) Patent Application Publication
(10) Pub. No.: US 2009/0239865 A1
(43) Pub. Date: Sep. 24, 2009

(54) DIBENZYL AMINE COMPOUNDS AND DERIVATIVES

(76) Inventors: George Chang, Old Saybrook, CT (US); Mary Didiuk, Madison, CT (US); Peter Dorff, Griswold, CT (US); Ravi Garigipati, South Glastonbury, CT (US); Wenhua Jiao, Salem, CT (US); Bruce Lefker, Gales Ferry, CT (US); David Perry, Mystic, CT (US); Roger Ruggeri, Waterford, CT (US); Toby Underwood, Kent (GB)

Correspondence Address: PFIZER INC.;PATENT DEPARTMENT, Bld 114 M/S 114, EASTERN POINT ROAD, GROTON, CT, 06340, US

(21) Appl. No.: 11/719,885

(22) PCT Filed: Nov. 21, 2005
(86) PCT No.: PCT/IB05/03500

§ 371 Date: May 22, 2007

Related U.S. Application Data
(60) Provisional application No. 60/630,434, filed on Nov. 23, 2004.
(60) Provisional application No. 60/715,617, filed on Sep. 12, 2005.

(51) Int. Cl. A61K 31/5377; C07D 257/06; A61K 31/41; A61K 31/4439; C07D 401/14; C07D 413/14; A61P 9/10

(52) U.S. Cl. 514/236.2; 548/251; 514/381; 514/340; 546/268.4; 544/140

(57) ABSTRACT

Dibenzyl amine compounds and derivatives, pharmaceutical compositions containing such compounds and the use of such compounds to elevate certain plasma lipid levels, including high density lipoprotein-cholesterol and to lower certain other plasma lipid levels, such as LDL-cholesterol and triglycerides and accordingly to treat diseases which are exacerbated by low levels of HDL cholesterol and/or high levels of LDL-cholesterol and triglycerides, such as atherosclerosis and cardiovascular diseases in some mammals, including humans.

### BACKGROUND OF INVENTION

This invention relates to dibenzyl amine compounds and derivatives, pharmaceutical compositions containing such compounds and their use to elevate certain plasma lipid levels, including high density lipoprotein (HDL)-cholesterol and to lower certain other plasma lipid levels, such as low density lipoprotein (LDL)-cholesterol and triglycerides and accordingly to treat diseases which are affected by low levels of HDL cholesterol and/or high levels of LDL-cholesterol and triglycerides, such as atherosclerosis and cardiovascular diseases in certain mammals (i.e., those which have CETP in their plasma), including humans.

Atherosclerosis and its associated coronary artery disease (CAD) is the leading cause of mortality in the industrialized world. Despite attempts to modify secondary risk factors (smoking, obesity, lack of exercise) and treatment of dyslipidemia with dietary modification and drug therapy, coronary heart disease (CHD) remains the most common cause of death in the U.S., where cardiovascular disease accounts for 44% of all deaths, with 53% of these associated with atherosclerotic coronary heart disease.

Risk for development of this condition has been shown to be strongly correlated with certain plasma lipid levels. While elevated LDL-C may be the most recognized form of dyslipidemia, it is by no means the only significant lipid associated contributor to CHD. Low HDL-C is also a known risk factor for CHD (Gordon, D. J., et al., “High-density Lipoprotein Cholesterol and Cardiovascular Disease”, Circulation, (1989), 79: 8-15).

High LDL-cholesterol and triglyceride levels are positively correlated, while high levels of HDL-cholesterol are negatively correlated with the risk for developing cardiovascular diseases. Thus, dyslipidemia is not a unitary risk profile for CHD but may be comprised of one or more lipid aberrations.

Among the many factors controlling plasma levels of these disease dependent principles, cholesteryl ester transfer protein (CETP) activity affects all three. The role of this 70,000 dalton plasma glycoprotein found in a number of animal species, including humans, is to transfer cholesteryl ester and triglyceride between lipoprotein particles, including high density lipoproteins (HDL), low density lipoproteins (LDL), very low density lipoproteins (VLDL), and chylomicrons. The net result of CETP activity is a lowering of HDL cholesterol and an increase in LDL cholesterol. This effect on lipoprotein profile is believed to be pro-atherogenic, especially in subjects whose lipid profile constitutes an increased risk for CHD.

No wholly satisfactory HDL-elevating therapies are on the market today. Niacin can significantly increase HDL, but has serious toleration issues which reduce compliance. Fibrates and the HMG CoA reductase inhibitors raise HDL-C, but in some patients, the result is an increase of modest proportions (˜10-12%). As a result, there is an unmet medical need for an approved therapeutic agent that elevates plasma HDL levels, thereby reversing or slowing the progression of atherosclerosis.

Thus, although there are a variety of anti-atherosclerosis therapies, there is a continuing need and a continuing search in this field of art for alternative therapies.

### SUMMARY OF THE INVENTION

This invention is directed to compounds according to Formula I

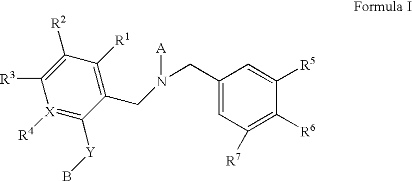

or a pharmaceutically acceptable salt of said compound, wherein

A is —COO(C₁-C₄)alkyl, cyano, —CHO, —CONH₂, —CO(C₁-C₄)alkyl, triazolyl, tetrazolyl, oxadiazolyl, isoxazolyl, pyrazolyl, or thiadiazolyl and A is optionally mono-, di- or tri-substituted with R⁰;

X is C or N, wherein if X is N, R⁴ is absent;

Y is a bond, —O—, —CR¹¹R¹²—, —CR¹¹R¹²—O—, or —O—CR¹¹R¹²—, wherein R¹¹ and R¹² are each independently hydrogen or (C₁-C₆)alkyl wherein said (C₁-C₆)alkyl is optionally substituted with one to nine halo, or R¹¹ and R¹² may be taken together to form a (C₃-C₆)cycloalkyl optionally substituted with one to nine halo;

B is aryl or heteroaryl wherein B is optionally mono-, di- or tri-substituted independently with (C₀-C₆)alkyl-NR⁸R⁹, (C₀-C₆)alkyl-CO—NR⁸R⁹, (C₀-C₆)alkyl-CO—OR¹⁰, (C₀-C₆)alkyl-NR¹³—CO—O—R¹⁰, (C₁-C₆)alkyl-O—CO—NR⁸R⁹, O—(C₁-C₆)alkyl-CO—O—R¹⁰, (C₀-C₆)alkyl-aryl, (C₀-C₆)alkyl-heteroaryl, O—(C₀-C₆)alkyl-aryl, O—(C₀-C₆)alkyl-heteroaryl, (C₀-C₆)alkyl-O-aryl, (C₀-C₆)alkyl-O-heteroaryl, halo, (C₂-C₆)alkenyl, (C₁-C₆)alkyl, hydroxy, (C₁-C₆)alkoxy, (C₁-C₄)alkylthio, nitro, cyano, oxo, (C₁-C₆)alkylcarbonyl, or (C₁-C₆)alkyloxycarbonyl wherein said (C₁-C₆)alkyl and (C₁-C₆)alkoxy substituents are each optionally substituted independently with one to nine halo, one or two hydroxy, one or two (C₁-C₆)alkoxy, one or two amino, one or two nitro, cyano, oxo, or carboxy, wherein R⁸ and R⁹ are each independently hydrogen, (C₁-C₆)alkyl, (C₁-C₆)alkoxy, or carboxy, R¹⁰ is hydrogen, (C₁-C₆)alkyl, or (C₁-C₆)alkoxy, and R¹³ is hydrogen or (C₁-C₆)alkyl wherein said (C₁-C₆)alkyl is optionally substituted with one to nine halo;

each R⁰ is independently hydrogen, halo, (C₁-C₆)alkyl, hydroxy, (C₁-C₆)alkoxy, amino, amido, cyano, oxo, carboxamoyl, carboxy, or (C₁-C₆)alkyloxycarbonyl, wherein said (C₁-C₆)alkyl substituent is optionally independently substituted with one or two oxo, one or two hydroxy, or one to nine halo; and

R¹, R², R³, R⁴, R⁵, R⁶, and R⁷ are each independently hydrogen, halo, cyano, hydroxy, (C₁-C₆)alkyl, (C₁-C₆)alkoxy, or (C₁-C₆)alkylthio wherein said (C₁-C₆)alkyl, (C₁-C₆)alkoxy, and (C₁-C₆)alkylthio substituents are each optionally substituted independently with one to nine halo, one or two cyano or one or two hydroxy.

The present invention is also directed to compounds of Formula I wherein

A is —COO(C₁-C₄)alkyl, cyano, —CHO, —CONH₂, —CO(C₁-C₄)alkyl, triazolyl, tetrazolyl, oxadiazolyl, isoxazolyl, pyrazolyl, or thiadiazolyl and A is mono-, di- or tri-substituted with R⁰;

X is C or N, wherein if X is N, R⁴ is absent;

Y is a bond, —O—, —CR¹¹R¹²—, —CR¹¹R¹²—O—, or —O—CR¹¹R¹²—, wherein R¹¹ and R¹² are each independently hydrogen or (C₁-C₆)alkyl wherein said (C₁-C₆)alkyl is optionally substituted with one to nine halo, or R¹¹ and R¹² may be taken together to form a (C₃-C₆)cycloalkyl optionally substituted with one to nine halo;

B is aryl or heteroaryl wherein B is optionally mono-, di- or tri-substituted independently with —(C₀-C₆)alkyl-NR⁸R⁹, —(C₀-C₆)alkyl-CO—NR⁸R⁹, —(C₀-C₆)alkyl-CO—OR¹⁰, —(C₀-C₆)alkyl-NR¹³—(C₀-C₆)alkyl-CO—O—R¹⁰, —(C₀-C₆)alkyl-NR¹³—(C₀-C₆)alkyl-CO—R¹⁴, —(C₀-C₆)alkyl-NR¹³—(C₀-C₆)alkyl-SO₂—R¹⁰, —(C₁-C₆)alkyl-O—CO—NR⁸R⁹, —O—C₁-C₆)alkyl-CO—O—R¹⁰, —(C₂-C₆)alkenyl-CO—O—R¹⁰, —(C₀-C₆)alkyl-aryl, —(C₀-C₆)alkyl-heteroaryl, —O—(C₀-C₆)alkyl-aryl, —O—(C₀-C₆)alkyl-heteroaryl, —(C₀-C₆)alkyl-O-aryl, —(C₀-C₆)alkyl-O-heteroaryl, —(C₀-C₆)alkyl-heterocycle, —O—(C₀-C₆)alkyl-heterocycle, —(C₀-C₆)alkyl-(C₃-C₆)cycloalkyl, —O—(C₀-C₆)alkyl-(C₃-C₆)cycloalkyl, —(C₀-C₆)alkyl-(C₃-C₆)cycloalkenyl, halo, (C₂-C₆)alkynyl, (C₂-C₆)alkenyl, (C₁-C₆)alkyl, hydroxy, (C₁-C₆)alkoxy, (C₁-C₄)alkylthio, nitro, cyano, oxo, —CO—(C₁-C₆)alkyl, or —CO—O—(C₁-C₆)alkyl wherein said aryl, heteroaryl, heterocycle, (C₃-C₆)cycloalkenyl, (C₃-C₆)cycloalkyl, (C₂-C₆)alkynyl, (C₂-C₆)alkenyl, (C₁-C₆)alkyl and (C₁-C₆)alkoxy substituents are each optionally substituted independently with one to nine halo, one or two hydroxy, one or two (C₁-C₆)alkoxy, one or two amino, one or two nitro, cyano, oxo, or carboxy, wherein R⁸ and R⁹ are each independently hydrogen, (C₁-C₆)alkyl, or (C₁-C₆)alkoxy, wherein said (C₁-C₆)alkyl is optionally substituted with one to nine halo; R¹⁰ is hydrogen, (C₁-C₆)alkyl, or (C₁-C₆)alkoxy, wherein said (C₁-C₆)alkyl is optionally substituted with one to nine halo; R¹³ is hydrogen or (C₁-C₆)alkyl wherein said (C₁-C₆)alkyl is optionally substituted with one to nine halo; and R¹⁴ is hydrogen, aryl, (C₁-C₆)alkyl, or (C₁-C₆)alkoxy wherein said (C₁-C₆)alkyl is optionally substituted with one to nine halo;

each R⁰ is independently hydrogen, halo, (C₁-C₆)alkyl, hydroxy, (C₁-C₆)alkoxy, amino, amido, cyano, oxo, carboxamoyl, carboxy, or (C₁-C₆)alkyloxycarbonyl, wherein said (C₁-C₆)alkyl substituent is optionally independently substituted with one or two oxo, one or two hydroxy, or one to nine halo; and

R¹, R², R³, R⁴, R⁵, R⁶, and R⁷ are each independently hydrogen, halo, cyano, hydroxy, (C₁-C₆)alkyl, (C₁-C₆)alkoxy, or (C₁-C₆)alkylthio wherein said (C₁-C₆)alkyl, (C₁-C₆)alkoxy, and (C₁-C₆)alkylthio substituents are each optionally substituted independently with one to nine halo, one or two cyano or one or two hydroxy.

The present invention is also directed to compounds of Formula V

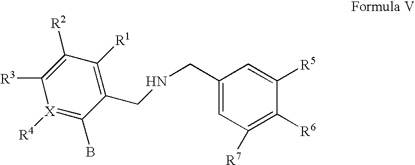

or a pharmaceutically acceptable salt of said compound; wherein
X is C or N, wherein if X is N, R⁴ is absent;

B is aryl or heteroaryl wherein B is optionally mono-, di- or tri-substituted independently with (C₀-C₆)alkyl-NR⁸R⁹, (C₀-C₆)alkyl-CO—NR⁸R⁹, (C₀-C₆)alkyl-CO—OR¹⁰, (C₀-C₆)alkyl-NR¹³—CO—O—R¹⁰, (C₁-C₆)alkyl-O—CO—NR⁸R⁹, O—(C₁-C₆)alkyl-CO—O—R¹⁰, (C₀-C₆)alkyl-aryl, (C₀-C₆)alkyl-heteroaryl, O—(C₀-C₆)alkyl-aryl, O—(C₀-C₆)alkyl-heteroaryl, (C₀-C₆)alkyl-O-aryl, (C₀-C₆)alkyl-O-heteroaryl, halo, (C₂-C₆)alkenyl, (C₁-C₆)alkyl, hydroxy, (C₁-C₆)alkoxy, (C₁-C₄)alkylthio, nitro, cyano, oxo, (C₁-C₆)alkylcarbonyl, or (C₁-C₆)alkyloxycarbonyl wherein said (C₁-C₆)alkyl and (C₁-C₆)alkoxy substituents are each optionally substituted independently with one to nine halo, one or two hydroxy, one or two (C₁-C₆)alkoxy, one or two amino, one or two nitro, cyano, oxo, or carboxy, wherein R⁸ and R⁹ are each independently hydrogen, (C₁-C₆)alkyl, (C₁-C₆)alkoxy, or carboxy, R¹⁰ is hydrogen, (C₁-C₆)alkyl, or (C₁-C₆)alkoxy, and R¹³ is hydrogen or (C₁-C₆)alkyl wherein said (C₁-C₆)alkyl is optionally substituted with one to nine halo;

each R⁰ is independently hydrogen, halo, (C₁-C₆)alkyl, hydroxy, (C₁-C₆)alkoxy, amino, amido, cyano, oxo, carboxamoyl, carboxy, or (C₁-C₆)alkyloxycarbonyl, wherein said (C₁-C₆)alkyl substituent is optionally independently substituted with one or two oxo, one or two hydroxy, or one to nine halo; and

R¹, R², R³, R⁴, R⁵, R⁶, and R⁷ are each independently hydrogen, halo, cyano, hydroxy, (C₁-C₆)alkyl, (C₁-C₆)alkoxy, or (C₁-C₆)alkylthio wherein said (C₁-C₆)alkyl, (C₁-C₆)alkoxy, and (C₁-C₆)alkylthio substituents are each optionally substituted independently with one to nine halo, one or two cyano or one or two hydroxy.

The present invention is also directed to compounds selected from the group consisting of:
N-[3,5-bis(trifluoromethyl)benzyl]-N-{[5′-isopropyl-2′-methoxy-4-(trifluoromethyl)biphenyl-2-yl]methyl}-2-methyl-2H-tetrazol-5-amine;
N-[3,5-bis(trifluoromethyl)benzyl]-N-{[2′-methoxy-5′-methyl-4-(trifluoromethyl)biphenyl-2-yl]methyl}-2-methyl-2H-tetrazol-5-amine;
Methyl-[3,5-bis(trifluoromethyl)benzyl]{[5′-isopropyl-2′-methoxy-4-(trifluoromethyl)biphenyl-2-yl]methyl}carbamate;
2′-{[[3,5-bis(trifluoromethyl)benzyl](2-methyl-2H-tetrazol-5-yl)amino]methyl}-6-methoxy-4′-(trifluoromethyl)biphenyl-3-carbaldehyde;
N-[3,5-bis(trifluoromethyl)benzyl]-N-{[2′-chloro-5′-methyl-4-(trifluoromethyl)biphenyl-2-yl]methyl}-2-methyl-2H-tetrazol-5-amine;
[2′-{[[3,5-bis(trifluoromethyl)benzyl](2-methyl-2H-tetrazol-5-yl)amino]methyl}-6-methoxy-4′-(trifluoromethyl)biphenyl-3-yl]acetonitrile;
N-[3,5-bis(trifluoromethyl)benzyl]-N-{[2′,5′-dimethoxy-4-(trifluoromethyl)biphenyl-2-yl]methyl}-2-methyl-2H-tetrazol-5-amine;
2′-{[[3,5-bis(trifluoromethyl)benzyl](2-methyl-2H-tetrazol-5-yl)amino]methyl}-6-methoxy-4′-(trifluoromethyl)biphenyl-3-carbonitrile;
N-[3,5-bis(trifluormethyl)benzyl]-N-{[5′-isopropyl-2′-methoxy-4-(trifluoromethyl)biphenyl-2-yl]methyl}acetamide;
N-[3,5-bis(trifluoromethyl)benzyl]-N-{[5′-fluoro-2′-methoxy-4-(trifluoromethyl)biphenyl-2-yl]methyl}-2-methyl-2H-tetrazol-5-amine;
N-[3,5-bis(trifluoromethyl)benzyl]-N-{[3′-isopropyl-4-(trifluoromethyl)biphenyl-2-yl]methyl}-2-methyl-2H-tetrazol-5-amine;
N-[3,5-bis(trifluoromethyl)benzyl]-N-{[2′-methoxy-4-(trifluoromethyl)biphenyl-2-yl]methyl}-2-methyl-2H-tetrazol-5-amine;
N-[3,5-bis(trifluoromethyl)benzyl]-N-{[2′-(methylthio)-4-(trifluoromethyl)biphenyl-2-yl]methyl}2-methyl-2H-tetrazol-5-amine;
N-[3,5-bis(trifluoromethyl)benzyl]-N-{[2′-(trifluoromethoxy)-4-(trifluoromethyl)biphenyl-2-yl]methyl}-2-methyl-2H-tetrazol-5-amine;
N-[3,5-bis(trifluoromethyl)benzyl]-N-{[2′-fluoro-5′-methyl-4-(trifluoromethyl)biphenyl-2-yl]methyl}-2-methyl-2H-tetrazol-5-amine;
N-[3,5-bis(trifluoromethyl)benzyl]-N-{[2′-methoxy-5′-[(4-methylpiperazin-1-yl)methyl]-4-(trifluoromethyl)biphenyl-2-yl]methyl}-2-methyl-2H-tetrazol-5-amine;
N-[3,5-bis(trifluoromethyl)benzyl]-N-[(5′-isopropyl-2′-methoxybiphenyl-2-yl)methyl]-2-methyl-2H-tetrazol-5-amine;
N-[3,5-bis(trifluoromethyl)benzyl]-N-{[5′-[(dimethylamino)methyl]-2′-methoxy-4-(trifluoromethyl)biphenyl-2-yl]methyl}-2-methyl-2H-tetrazol-5-amine;
Methyl-N-{[2′-{[[3,5-bis(trifluoromethyl)benzyl](2-methyl-2H-tetrazol-5-yl)amino]methyl}-6-methoxy-4′-(trifluoromethyl)biphenyl-3-yl]methyl}-N-methylglycinate;
N-[3,5-bis(trifluoromethyl)benzyl]-N-{[2′-ethoxy-4-(trifluoromethyl)biphenyl-2-yl]methyl}-2-methyl-2H-tetrazol-5-amine;
1-[2′-{[[3,5-bis(trifluoromethyl)benzyl](2-methyl-2H-tetrazol-5-yl)amino]methyl}-6-fluoro-4′-(trifluoromethyl)biphenyl-3-yl]ethanone; and
4-{1-[2-{[[3,5-bis(trifluoromethyl)benzyl](2-methyl-2H-tetrazol-5-yl)amino]methyl}-4-(trifluoromethyl)phenyl]propoxy}benzamide;
   or a prodrug thereof, or a pharmaceutically acceptable salt of said compound or of said prodrug.

In addition, the present invention provides pharmaceutical compositions which comprise a therapeutically effective amount of a compound of the present invention, or a pharmaceutically acceptable form of said compound and a pharmaceutically acceptable vehicle, diluent or carrier.

In addition, the present invention provides pharmaceutical compositions for the treatment of atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal which comprise a therapeutically effective amount of a compound of the present invention, or a pharmaceutically acceptable form of said compound and a pharmaceutically acceptable vehicle, diluent or carrier.

Moreover, the present invention provides pharmaceutical combination compositions comprising:

a therapeutically effective amount of a composition comprising

a first compound, said first compound being a compound of the present invention, or a pharmaceutically acceptable form of said compound;

a second compound, said second compound being an HMG CoA reductase inhibitor, an MTP/Apo B secretion inhibitor, a PPAR modulator, a bile acid reuptake inhibitor, a cholesterol absorption inhibitor, a cholesterol synthesis inhibitor, a fibrate, niacin, an antihypertensive, slow-release niacin, a combination of niacin and lovastatin, an ion-exchange resin, an antioxidant, an ACAT inhibitor or a bile acid sequestrant (preferably an HMG-CoA reductase inhibitor, a PPAR modulator, fenofibrate, gemfibrozil, lovastatin, simvastatin, pravastatin, fluvastatin, atorvastatin, rivastatin, rosuvastatin or pitavastatin); and

a pharmaceutical vehicle, diluent or carrier. This composition may be used to treat the aforementioned diseases, including atherosclerosis.

Also, the present invention provides a kit for achieving a therapeutic effect in a mammal comprising packaged in association a first therapeutic agent comprising a therapeutically effective amount of a compound of the present invention, a prodrug thereof, or a pharmaceutically acceptable salt of said compound or of said prodrug and a pharmaceutically acceptable carrier, a second therapeutic agent comprising a therapeutically effective amount of an HMG CoA reductase inhibitor, a PPAR modulator, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant and a pharmaceutically acceptable carrier and directions for administration of said first and second agents to achieve the therapeutic effect.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

Before the present compounds, compositions and methods are disclosed and described, it is to be understood that this invention is not limited to specific synthetic methods of making that may of course vary. It is also to be understood that the terminology used herein is for the purpose of describing particular embodiments only and is not intended to be limiting.

The present invention also relates to the pharmaceutically acceptable acid addition salts of compounds of the present invention. The acids which are used to prepare the pharmaceutically acceptable acid addition salts of the aforementioned base compounds of this invention are those which form non-toxic acid addition salts, (i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The invention also relates to base addition salts of the compounds of the present invention. The chemical bases that may be used as reagents to prepare pharmaceutically acceptable base salts of those compounds of the present invention that are acidic in nature are those that form non-toxic base salts with such compounds. Such non-toxic base salts include, but are not limited to those derived from such pharmacologically acceptable cations such as alkali metal cations (e, potassium and sodium) and alkaline earth metal cations (e calcium and magnesium), ammonium or water-soluble amine addition salts such as N-methylglucamine-(meglumine), and the lower alkanolammonium and other base salts of pharmaceutically acceptable organic amines.

The chemist of ordinary skill will recognize that certain compounds of this invention will contain one or more atoms which may be in a particular stereochemical or geometric configuration, giving rise to stereoisomers and configurational isomers. All such isomers and mixtures thereof are included in this invention. Hydrates and solvates of the compounds of this invention are also included.

Where the compounds of the present invention possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form.

The compounds of this invention may contain olefin-like double bonds. When such bonds are present, the compounds of the invention exist as cis and trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

Alpha and Beta refer to the orientation of a substituent with reference to the plane of the ring. Beta is above the plane of the ring and Alpha is below the plane of the ring.

This invention also includes isotopically-labeled compounds, which are identical to those described by formulas I and II, except for the fact that one or more atoms are replaced by one or more atoms having specific atomic mass or mass numbers. Examples of isotopes that can be incorporated into compounds of the invention include isotopes of hydrogen, carbon, nitrogen, oxygen, sulfur, fluorine, and chlorine such as ²H, ³H, ¹³C, ¹⁴C, ¹⁵N, ¹⁸O, ¹⁷O, ¹⁸F, and ³⁶Cl respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out the procedures disclosed in the schemes and/or in the Examples below, by substituting a readily available isotopically labeled reagent for a non-isotopically labeled reagent.

In this specification and in the claims that follow, reference will be made to a number of terms that shall be defined to have the following meanings:

As used herein, the term mammals is meant to refer to all mammals which contain CETP in their plasma, for example, rabbits and primates such as monkeys and humans, including males and females. Certain other mammals e.g., dogs, cats, cattle, goats, sheep and horses do not contain CETP in their plasma and so are not included herein.

The term “treating”, “treat” or “treatment” as used herein includes preventative (e.g., prophylactic) and palliative treatment.

By “pharmaceutically acceptable” is meant the carrier, diluent, excipients, and/or salt must be compatible with the other ingredients of the formulation, and not deleterious to the recipient thereof.

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. By “tautomers” is meant chemical compounds that may exist in two or more forms of different structure (isomers) in equilibrium, the forms differing, usually, in the position of a hydrogen atom. Various types of tautomerism can occur, including keto-enol, ring-chain and ring-ring tautomerism. The expression “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the compounds of the present invention include but are not limited to those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

The following paragraphs describe exemplary ring(s) for the generic ring descriptions contained herein.

By “halo” or “halogen” is meant chloro, bromo, iodo, or fluoro.

By “alkyl” is meant straight chain saturated hydrocarbon or branched chain saturated hydrocarbon. Exemplary of such alkyl groups (assuming the designated length encompasses the particular example) are methyl, ethyl, propyl, isopropyl, butyl, sec-butyl, tertiary butyl, isobutyl, pentyl, isopentyl, neopentyl, tertiary pentyl, 1-methylbutyl, 2-methylbutyl, 3-methylbutyl, hexyl, isohexyl, heptyl and octyl.

“Alkenyl” referred to herein may be linear or branched, and they may also be cyclic (e.g. cyclobutenyl, cyclopentenyl, cyclohexenyl) or bicyclic or contain cyclic groups. They contain 1-3 carbon-carbon double bonds, which can be cis or trans.

By “alkoxy” is meant straight chain saturated alkyl or branched chain saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

The term “aryl” means a carbocyclic aromatic system containing one, two or three rings wherein such rings may be fused. The term “fused” means that a second ring is present (ie, attached or formed) by having two adjacent atoms in common (ie, shared) with the first ring. The term “fused” is equivalent to the term “condensed”. The term “aryl” embraces aromatic radicals such as phenyl, naphthyl, tetrahydronaphthyl, indane and biphenyl.

The term “heteroaryl” means a carbocyclic aromatic system containing one, two, three or four heteroatoms selected independently from oxygen, nitrogen and sulfur and having one, two or three rings wherein such rings may be fused. The term “fused” means that a second ring is present (ie, attached or formed) by having two adjacent atoms in common (ie, shared) with the first ring. The term “fused” is equivalent to the term “condensed”. The term “heteroaryl” embraces aromatic radicals such as quinolinyl, benzofuranyl, benzodioxanyl, piprazinyl, imidazolyl, triazolyl, tetrazolyl, oxazolyl, oxadiazolyl, isoxazolyl, pyrazolyl, thiazolyl and thiadiazolyl.

The term “heterocycle” means a nonaromatic carbocyclic system containing one, two, three or four heteroatoms selected independently from oxygen, nitrogen and sulfur and having one, two or three rings wherein such rings may be fused, wherein fused is defined above. The term “heterocycle” includes but is not limited to lactones, lactams, cyclic ethers and cyclic amines, including the following exemplary ring systems: epoxide, tetrahydrofuran, tetrahydropyran, dioxane, aziridines, pyrrolidine, piperidine, and morpholine.

It is to be understood that if a carbocyclic or heterocylic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3- or 4-pyridyl, the term “thienyl” means 2- or 3-thienyl, and so forth.

As used herein, the expressions “reaction-inert solvent” and “inert solvent” refer to a solvent or a mixture thereof which does not interact with starting materials, reagents, intermediates or products in a manner which adversely affects the yield of the desired product.

In one embodiment of the compounds of the present invention, X is C.

In another embodiment, A is

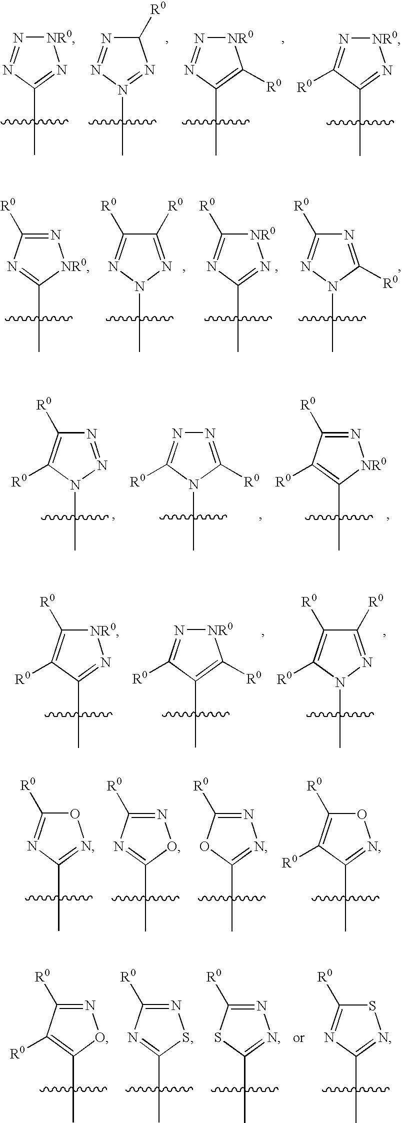

wherein each R⁰ is independently hydrogen, (C₁-C₃)alkyl, (C₁-C₃)alkoxy, hydroxy, or halo, wherein the alkyl or alkoxy is optionally independently substituted with one to nine halo or hydroxy.
In another embodiment, A is

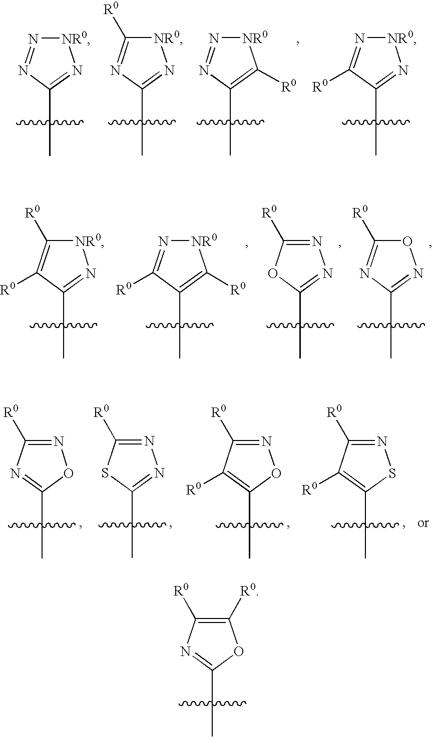

In another embodiment, A is

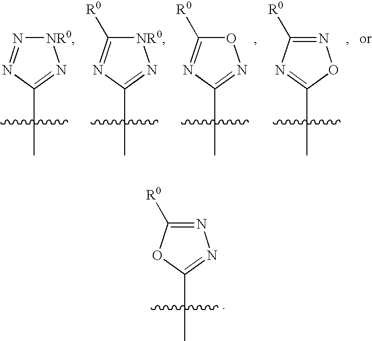

in another embodiment, A is —COOCH₂CH₃, —COOCH₃, cyano, —CHO, —CONH₂, —COCH₂CH₃, or —COCH₃

In another embodiment, R⁰ is independently hydrogen, (C₁-C₃)alkyl, or (C₁-C₃)alkoxy, wherein the alkyl or alkoxy is optionally independently substituted with one to nine halo or hydroxyl. In another embodiment, R⁰ is hydrogen, CH₃ or CF₃.
B is

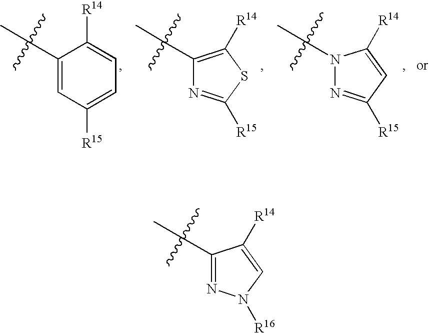

wherein R¹⁴ is halo, cyano, (C₁-C₆)alkyl or —O—(C₁-C₆)alkyl wherein said alkyl substituent is optionally substituted with one to four fluorines; R¹⁵ is —(C₀-C₆)alkyl-NR⁸R⁹, —(C₀-C₆)alkyl-CO—OR¹⁰, —(C₀-C₆)alkyl-NR¹³—(C₀-C₆)alkyl-CO—O—R¹⁰, —(C₁-C₆)alkyl-O—CO—NR⁸R⁹, —O—(C₁-C₆)alkyl-CO—O—R¹⁰, —(C₀-C₆)alkyl-heterocycle, —(C₀-C₆)alkyl-1-tetrazolyl, halo, (C₁-C₆)alkyl, (C₁-C₆)alkoxy, cyano, —CO—(C₁-C₆)alkyl, or —CO—O—(C₀-C₆)alkyl, wherein said alkyl and alkoxy substituents each optionally substituted independently with one to four fluorines or one or two hydroxyl; and R¹⁶ is —(C₀-C₆)alkyl-CO—OR¹⁰, —(C₂-C₆)alkyl-NR¹³—CO—O—R¹⁰, —(C₂-C₆)alkyl-O—CO—NR⁸R⁹, —(C₀-C₆)alkyl-1-tetrazolyl, (C₁-C₆)alkyl, or —CO—(C₁-C₆)alkyl, wherein said alkyl substituent is optionally substituted with one to four fluorines or one or two hydroxyl.

In another embodiment, B is phenyl or pyridyl optionally mono- or di-substituted independently with —(C₀-C₆)alkyl-NR⁸R⁹, —(C₀-C₆)alkyl-CO—OR¹⁰, —(C₀-C₆)alkyl-NR¹³—(C₀-C₆)alkyl-CO—O—R¹⁰, —(C₁-C₆)alkyl-O CO—NR⁸R⁹, —O—(C₁-C₆)alkyl-CO—O—R¹⁰, —(C₀-C₆)alkyl-heterocycle, —(C₀-C₆)alkyl-1-tetrazolyl, halo, (C₁-C₆)alkyl, (C₁-C₆)alkoxy, cyano, —CO—(C₁-C₆)alkyl, or —CO—O—(C₀-C₆)alkyl, wherein said alkyl and alkoxy substituents each optionally substituted independently with one to four fluorines or one or two hydroxyl.

In another embodiment, Y is a bond.

In another embodiment, R¹ and R⁶ are each hydrogen; R⁴ is absent or is hydrogen; and R², R³, R⁵, and R⁷ are each independently hydrogen, cyano, (C₁-C₆)alkyl or (C₁-C₆)alkoxy wherein said (C₁-C₆)alkyl and (C₁-C₆)alkoxy substituents each are optionally substituted independently with one to nine fluorines.

In another embodiment, R², R³, R⁵, and R⁷ are each hydrogen, methyl, cyano, or CF₃.

In another embodiment, X is C; R¹, R⁴, and R⁶ are each hydrogen; R², R³, R5, and R⁷ are each independently hydrogen, cyano, (C₁-C₆)alkyl or (C₁-C₆)alkoxy wherein said (C₁-C₆)alkyl and (C₁-C₆)alkoxy substituents each are optionally substituted independently with one to nine fluorines; and A is —COOCH₂CH₃, —COOCH₃, cyano, —CHO, —CONH₂, —COCH₂CH₃, —COCH₃, wherein each R⁰ is independently hydrogen, (C₁-C₃)alkyl, (C₁-C₃)alkoxy, hydroxy, or halo, wherein the alkyl or alkoxy is optionally independently substituted with one to nine halo or hydroxy.

In another embodiment, B is phenyl optionally mono- or di-substituted independently with NR⁸R⁹, (C₀-C₆)alkyl-CO—OR¹⁰, (C₀-C₆)alkyl-NR¹³—CO—O—R¹⁰, (C₀-C₆)alkyl-O—CO—NR⁸R⁹, O—(C₀-C₆)alkyl-CO—O—R¹⁰, (C₀-C₆)alkyl-1-tetrazolyl, halo, (C₁-C₆)alkyl, (C₁-C₆)alkoxy, cyano, (C₁-C₆)alkylcarbonyl, or (C₁-C₆)alkyloxycarbonyl, wherein said (C₁-C₆)alkyl and (C₁-C₆)alkoxy substituents each optionally substituted independently with one to four fluorines or one or two hydroxy; and A is —COOCH₂CH₃, —COOCH₃, cyano, —CHO, —CONH₂, —COCH₂CH₃, —COCH₃,

In one embodiment of the method of the present invention, atherosclerosis is treated.

In another embodiment of the method of the present invention, peripheral vascular disease is treated.

In another embodiment of the method of the present invention, dyslipidemia is treated.

In another embodiment of the method of the present invention, hyperbetalipoproteinemia is treated.

In another embodiment of the method of the present invention, hypoalphalipoproteinemia is treated.

In another embodiment of the method of the present invention, familial-hypercholesterolemia is treated.

In another embodiment of the method of the present invention, coronary artery disease is treated.

In another embodiment of the method of the present invention, myocardial infarction is treated.

In one embodiment of the combination or kit of the present invention, the second compound is an HMG-CoA reductase inhibitor or a PPAR modulator.

In another embodiment of the combination or kit of the present invention, the second compound is fenofibrate, gemfibrozil, lovastatin, simvastatin, pravastatin, fluvastatin, atorvastatin, rivastatin, rosuvastatin or pitavastatin.

In another embodiment of the combination or kit of the present invention, the combination further comprising a cholesterol absorption inhibitor, wherein the cholesterol absorption inhibitor may be ezetimibe.

In general, the compounds of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section.

Analogous processes are disclosed in the following U.S. patents, which are hereby incorporated by reference herein in their entirety: U.S. Pat. No. 6,140,342; U.S. Pat. No. 6,362,198; U.S. Pat. No. 6,147,090; U.S. Pat. No. 6,395,751; U.S. Pat. No. 6,147,089; U.S. Pat. No. 6,310,075; U.S. Pat. No. 6,197,786; U.S. Pat. No. 6,140,343; U.S. Pat. No. 6,489,478; and International Publication No. WO 00/17164.

The Reaction Schemes herein described are intended to provide a general description of the methodology employed in the preparation of many of the Examples given. However, it will be evident from the detailed descriptions given in the Experimental section that the modes of preparation employed extend further than the general procedures described herein. In particular, it is noted that the compounds prepared according to these Schemes may be modified further to provide new Examples within the scope of this invention. For example, an ester functionality may be reacted further using procedures well known to those skilled in the art to give another ester, an amide, a carbinol or a ketone.

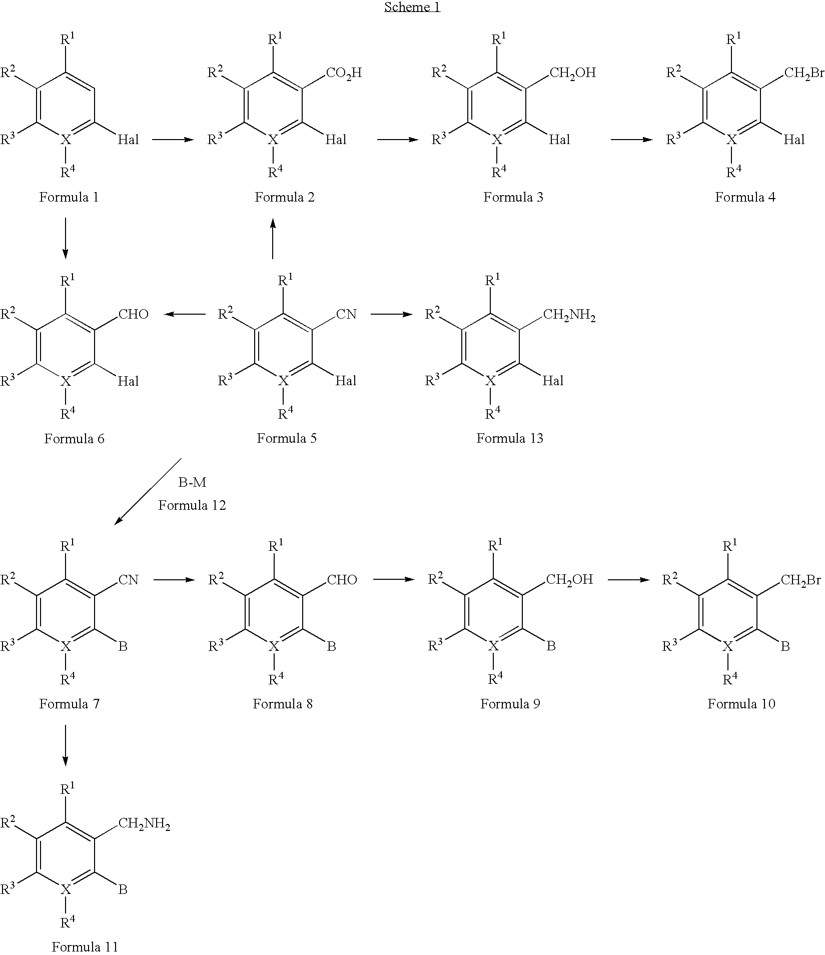

According to reaction Scheme 1, desired intermediate compounds in Scheme 1, wherein Hal is a halogen, and B, X, R¹, R², R³, and R⁴ are as described above may be prepared from compounds of Formula 2 and Formula 6, which are commercially available. Compounds of Formulas 2 and 6 may be prepared by methods known to those skilled in the art such as by directed metallation chemistry and trapping with a suitable electrophile such as carbon dioxide, dimethyl formamide (DMF), or N-formylmorpholine. More specifically, treatment of compounds of Formula 1 with 1-lithium-2,2,6,6-tetramethylpiperidine and quenching with Carbon Dioxide (F. Mongin, O. Desponds, M. Schlosser* (Tetrahedron Letters, Vol. 37, No 16, pp 2767-2770, 1996) at low temperature, preferably between −100° C. and −78° C., in a polar aprotic solvent such as ether or tetrahydrofuran (THF), preferably THF at −100° C. yields compounds of Formulas 2 and 6. Alternatively, compounds of Formulas 2 and 6 may be prepared by hydrolysis of compounds of Formula 5, which are commercially available, or may be prepared by methods known to those skilled in the art, with a suitable acid such as sulphuric acid.

As shown in scheme 1, compounds of Formula 7 may be prepared by transition metal cross coupling of compounds with Formula 5 and Formula 12 using a variety of conditions wherein M in Formula 12 refer to species such as —B(OH)₂, —B(OR)₂, Zn-halides, and —SnR₃. Suzuki cross coupling with aryl boronic acids is preferred. For general reference see A. Suzuki, H. C. Brown. Organic Syntheses via Boranes. Vol 3, Suzuki Coupling. Aldrich Chemical Company© 2003. When Hal=Cl, it is preferable to use modified Suzuki conditions (see Fu et al. J. Am. Chem. Soc., 2000, 122, 4020-4028). The preferred catalyst is tris(dibenzylideneaceton)dipalladium(0) with tert-butylphosphine.tetrafluoroborate adduct. The preferred solvent is dioxan with potassium fluoride as the preferred base at a temperature between 20° C. and 120° C. preferably between 60° C. and 110° C.

As shown in scheme 1, compounds of Formula 8 may be prepared by reduction of compounds of Formula 7 with a suitable hydride reducing agent, preferably diisobutylaluminum hydride in a suitable solvent such as THF, dioxane, methylene chloride. The preferred solvent is THF at a temperature between −78° C. and 68° C., preferably −10-20° C.

As shown in scheme 1, compounds of Formulas 3 and 9 may prepared by reduction of the compounds of Formula 2, Formula 6, or compounds of Formula 8 with a suitable reducing agent such as lithium aluminium hydride (LAH), sodium borohydride or borane-tetrahydrofuran complex in a solvent such as dioxan, methylene chloride, ethanol or THF. The preferred reducing agent for reduction of compounds of Formula 2 was Borane-Tetrahydrofuran complex, and the preferred solvent THF at a temperature between −78 and 100° C. preferably at 0-50° C. The preferred reducing agent of compounds of Formulas 6 and 8 is sodium borohydride, and preferred solvent is ethanol at a temperature between −78 and 100° C., preferably 0-50° C.

As shown in scheme 1, compounds of Formulas 4 and 10 may be prepared by brominating compounds of Formulas 3 or 9 respectively using a suitable brominating agent such as tribromophosphine or a combination of carbon tetrabromide and triphenylphosphine in an inert solvent such as methylene chloride, THF, or dioxan. The preferred brominating agent is a combination of carbon tetrabromide and triphenylphosphine, and the preferred solvent is methylene chloride at a temperature between −78° C. and 100° C., preferably −10° C.-20° C.

As shown in scheme 1, compounds of Formulas 13 and 11 may be prepared by reduction or hydrogenation of compounds of Formulas 5 or 7 respectively using a suitable reducing agent such as LAH, or a suitable hydrogenation catalyst such as palladium on carbon or palladium hydroxide. The reducing agent of choice is LAH in a suitable solvent such as THF, methylene chloride, or dioxan. The solvent of choice is THF at a temperature between −78° C. and 68° C., preferably −78° C.-40° C.

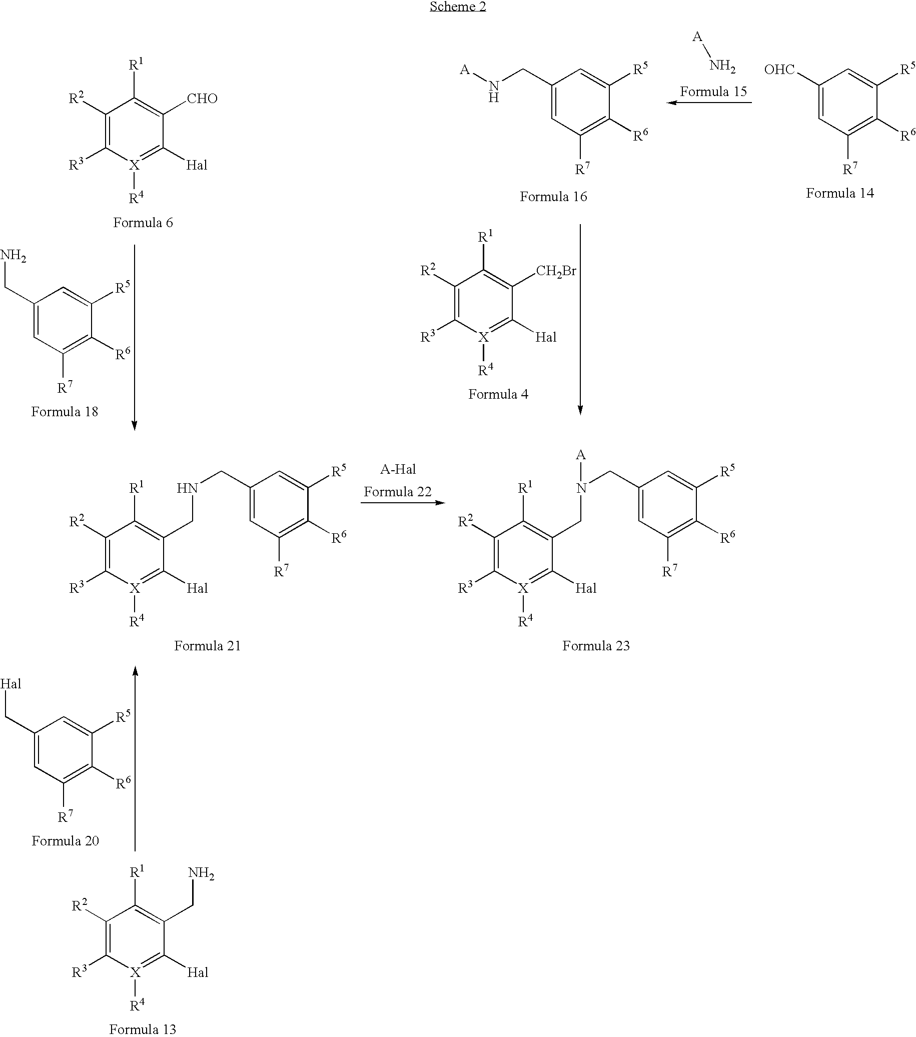

According to reaction Scheme 2, desired compounds depicted as Formula 23 in Scheme 2, wherein Hal is a halogen and A, X, R¹, R², R³, R⁴, R⁵, R⁶ and R⁷ are as described above may be prepared by alkylation of compounds of Formula 16 and Compounds of Formula 4 with a suitable base such as sodium hydride, potassium-tert-butoxide or metallated hexamethyldisilazine in a suitable polar solvent such as THF, dimethylformamide, or N-methylpyrrolidinone. The base of choice is potassium-tert-butoxide, and the preferred solvent is THF at a temperature between 0° C. and 67° C., preferably 20° C.-67° C.

Compounds of Formula 16 may be prepared by reductive amination of compounds of aldehydes of Formula 14 with amines of Formula 15 and a suitable reducing agent such as sodium borohydride, sodium triacetoxyborohydride, or sodium cyanoborohydride, in a suitable solvent such as THF, methylene chloride, dioxan, or toluene. The method of choice is imine formation in the presence of 4 Å Molecular Sieves in toluene at a temperature between 20° C. and 111° C., preferably 100° C.-111° C., followed by removal of the solvent, dissolution of the residue in a polar solvent, preferably ethanol, then reduction with a suitable hydride reducing agent, preferably sodium borohydride, at a temperature between 0° C. and 78° C., preferably 20° C.-50° C.

Alternatively, compounds of Formula 23 may be prepared by alkylation or acylation of compounds of Formula 21 with compounds of Formula 22 using a suitable base such as triethylamine, diisopropylethylamine, potassium carbonate, or sodium carbonate. The preferred base is diisopropylethylamine in a suitable inert solvent such as THF, methylene chloride, or dioxan. The preferred solvent is methylene chloride at a temperature between −40° C. and 40° C., preferably 0-20° C.

Compounds of Formula 21 may be prepared by reductive amination of compounds of Formula 6 and compounds of Formula 18 with a suitable reducing agent such as sodium borohydride, sodium triacetoxyborohydride, or sodium cyanoborohydride. The preferable reducing agent is sodium borohydride in a suitable solvent such as ethanol, THF, methylene chloride, dioxan, or toluene. The preferred solvent is ethanol at a temperature of −78° C. and 67° C. preferably 0-50° C.

Alternatively, compounds of Formula 21 may be prepared by alkylation of compounds of Formula 13 and compounds of Formula 20 using a suitable base such as triethylamine, diisopropylethylamine, potassium carbonate, or sodium carbonate. The preferred base is diisopropylethylamine in a suitable inert solvent such as THF, methylene chloride, or dioxan. The preferred solvent is methylene chloride at a temperature between −40° C. and 40° C., preferably 0-20° C.

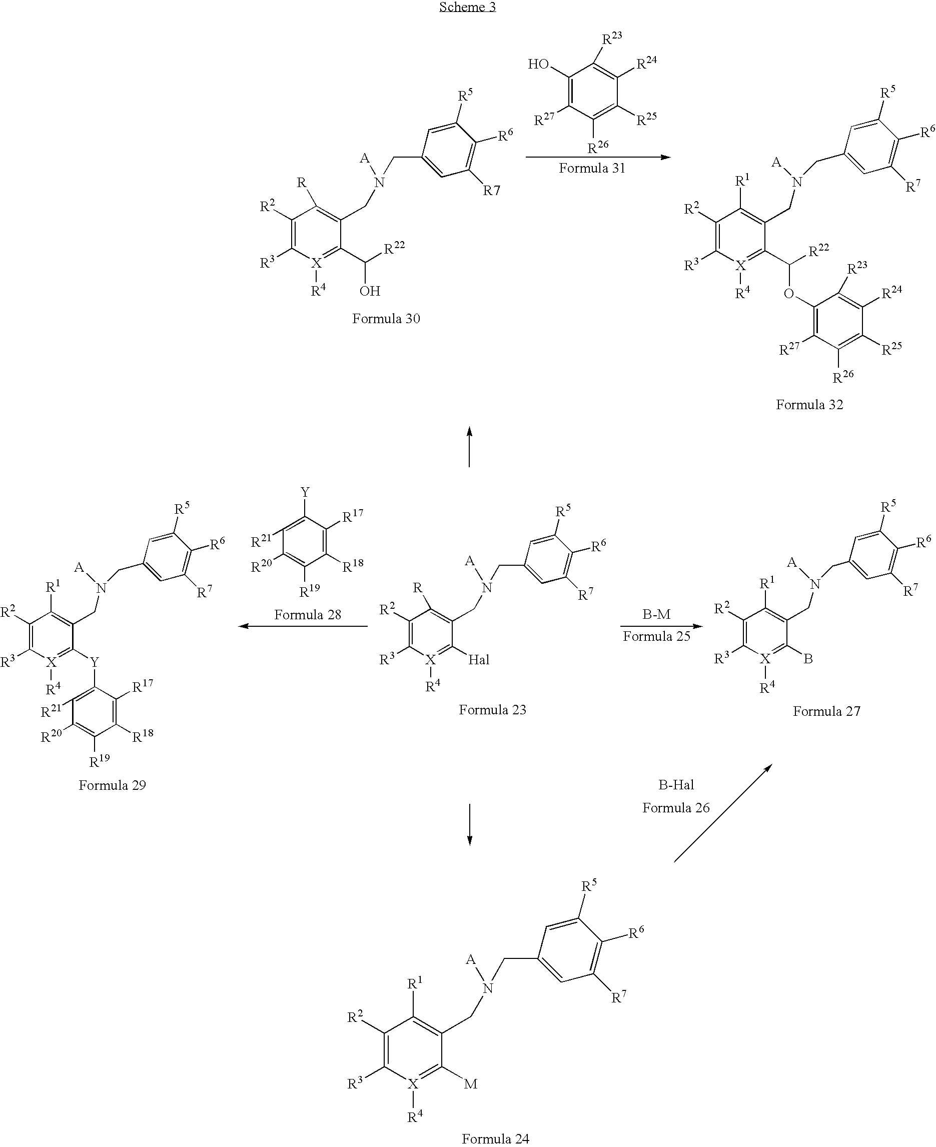

According to reaction Scheme 3, desired compounds depicted as Formula 24, wherein A, B, X, Y, R¹, R², R³, R⁴, R⁵, R⁶ and R⁷ are as described above, R²² is R¹¹ or R¹² as described above, and R¹⁷, R¹⁸, R¹⁹, R²⁰, R²¹, R²³, R²⁴, R²⁵, R²⁶, and R²⁷ are the optional substitutents of B as described above may be prepared from compounds of Formula 23 by a range of metallations by those skilled in the art, such as lithium-halogen exchange with ^{t}BuLi, ⁿBuLi or ^{l}PrMgCl followed by quenching with trimethylborate and acid hydrolysis to the boronic acid. Alternatively, transition metal assisted coupling can be employed. The method of choice is coupling of bis(pinacolato)diboron using a suitable catalyst such as Pd(OAc)₂, Pd₂ dba₃ or PdCl₂(dppf)₂, preferably PdCl₂(dppf)₂ in a suitable solvent such as dioxan, dimethyl sulfoxide, DMF, NMP preferably dimethyl sulfoxide with a suitable base such as KOAc, Na₂CO₃ or K₂CO₃ preferably KOAc as described in Miyaura et al, JOC, 1995, 60, p 7508.

Compounds of Formula 27 may be prepared by transition metal cross coupling of compounds with Formula 23 and Formula 24 with compounds of Formula 25 and Formula 26 respectively using a variety of conditions where B in compounds of Formula 25 and Formula 26 wherein Hal is a halogen and M refer to species such as —B(OH)₂, —B(OR)₂, Zn-halides, —SnR₃. Suzuki cross coupling with aryl boronic acids is preferred as described in A. Suzuki, H. C. Brown. Organic Syntheses via Boranes. Vol 3, Suzuki Coupling. Aldrich Chemical Company© 2003. The preferred catalyst is tetrakis(triphenylphosphine)palladium(0). The preferred solvent is dioxan/ethanol 2:1 with sodium carbonate in water as the preferred base at a temperature between 20° C. and 102° C., preferably between 60° C. and 102° C.

As shown in scheme 3, compounds of Formula 29 may be prepared by synthesis from compounds of Formula 23 and compounds of Formula 28 with a suitable base such as potassium carbonate, sodium carbonate or cesium carbonate. The preferred base where Y is an oxygen linker is cesium carbonate in a suitable solvent such as dimethylforamide or N-methylpyrrolidinone. The solvent of choice is dimethylformamide at a temperature between 20° C. and 153° C., preferably 40-110° C. As shown in scheme 3, compounds of Formula 30 may be prepared by metal halogen exchange of compounds of Formula 23 using a suitable metalating agent such as butyl lithium, magnesium or isopropyl magnesium chloride. The metalating agent of choice is isopropyl magnesium chloride in a suitable inert solvent such as THF, ether or Dioxan. The preferred solvent is THF at a temperature of −78° C. to 67° C., preferably 0-20° C. as described by Garst et al Coordination Chemistry Reviews 248 (2004) 623-652.

As shown in scheme 3, compounds of Formula 32 may be prepared by ether formation of compounds of Formula 30 and Formula 31 using Mitsunobu conditions as described by O. Mitsunobo Synthesis vol 1, (1981) 1-29. The preferred reagents are a combination of triphenylphosphine and diisopropylcarbodiimide in a suitable solvent such as ether, THF, or dioxan. The preferred solvent is THF at a temperature between −10° C. and 67° C., preferably 0-20° C.

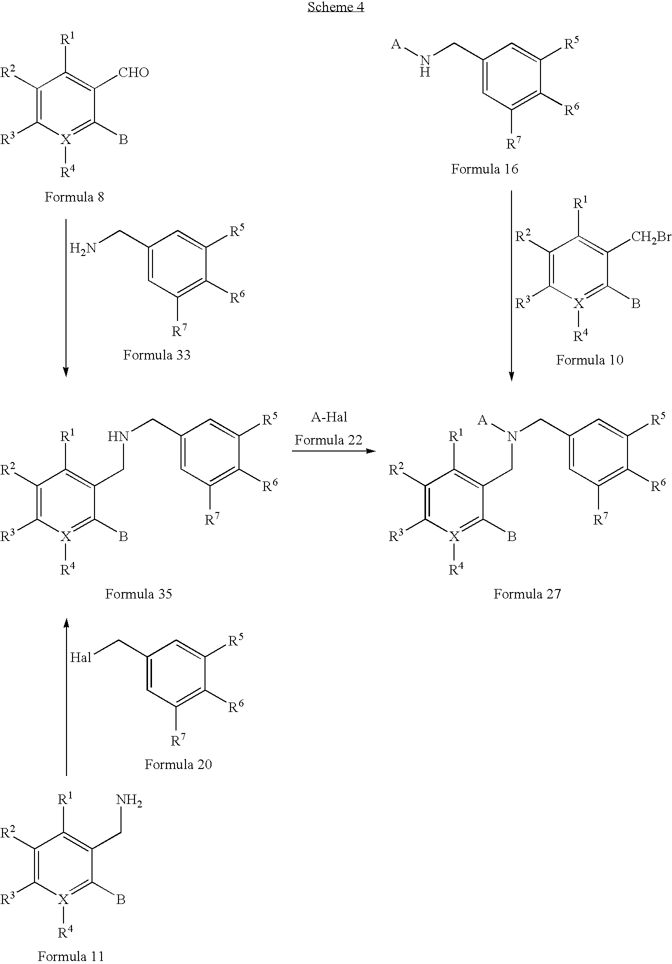

According to reaction Scheme 4, desired compounds of Formula 35, wherein A, B, X, R¹, R², R³, R⁴, R⁵, R⁶ and R⁷ are as described above may be prepared by reductive aminations of compounds of Formula 8 and Formula 33 using a suitable reducing agent such as sodium borohydride, sodium triacetoxyborohydride, or sodium cyanoborohydride. The preferable reducing agent is sodium borohydride in a suitable solvent such as ethanol, THF, methylene chloride, dioxan, or toluene. The preferred solvent is ethanol at a temperature of −78° C. and 67° C. preferably 0-50° C.

Alternatively, compounds of Formula 35 may be prepared by alkylation of compounds of Formula 11 with compounds of Formula 20 using a suitable base such as triethylamine, diisopropylethylamine, potassium carbonate, or sodium carbonate. The preferred base is diisopropylethylamine in a suitable inert solvent such as THF, methylene chloride, or dioxan. The preferred solvent is methylene chloride at a temperature between −40° C. and 40° C., preferably 0-20° C.

Compounds of Formula 27 may be prepared by alkylation of compounds of Formula 10 with compounds of Formula 16 using a suitable base such as sodium hydride, potassium-tert-butoxide or metallated hexamethyldisilazine in a suitable polar solvent such as THF, dimethylformamide, N-methylpyrrolidinone. The base of choice is potassium-tert-butoxide, and the preferred solvent is THF at a temperature between 0° C. and 67° C., preferably 20° C.-67° C.

Alternatively, compounds of Formula 27 may be prepared by alkylation or acylation of compounds of Formula 35 and compounds of Formula 22 using a suitable base such as triethylamine, diisopropylethylamine, potassium carbonate, or sodium carbonate. The preferred base is diisopropylethylamine in a suitable inert solvent such as THF, methylene chloride, or dioxan. The preferred solvent is methylene chloride at a temperature between 40° C. and 40° C., preferably 0-20° C.

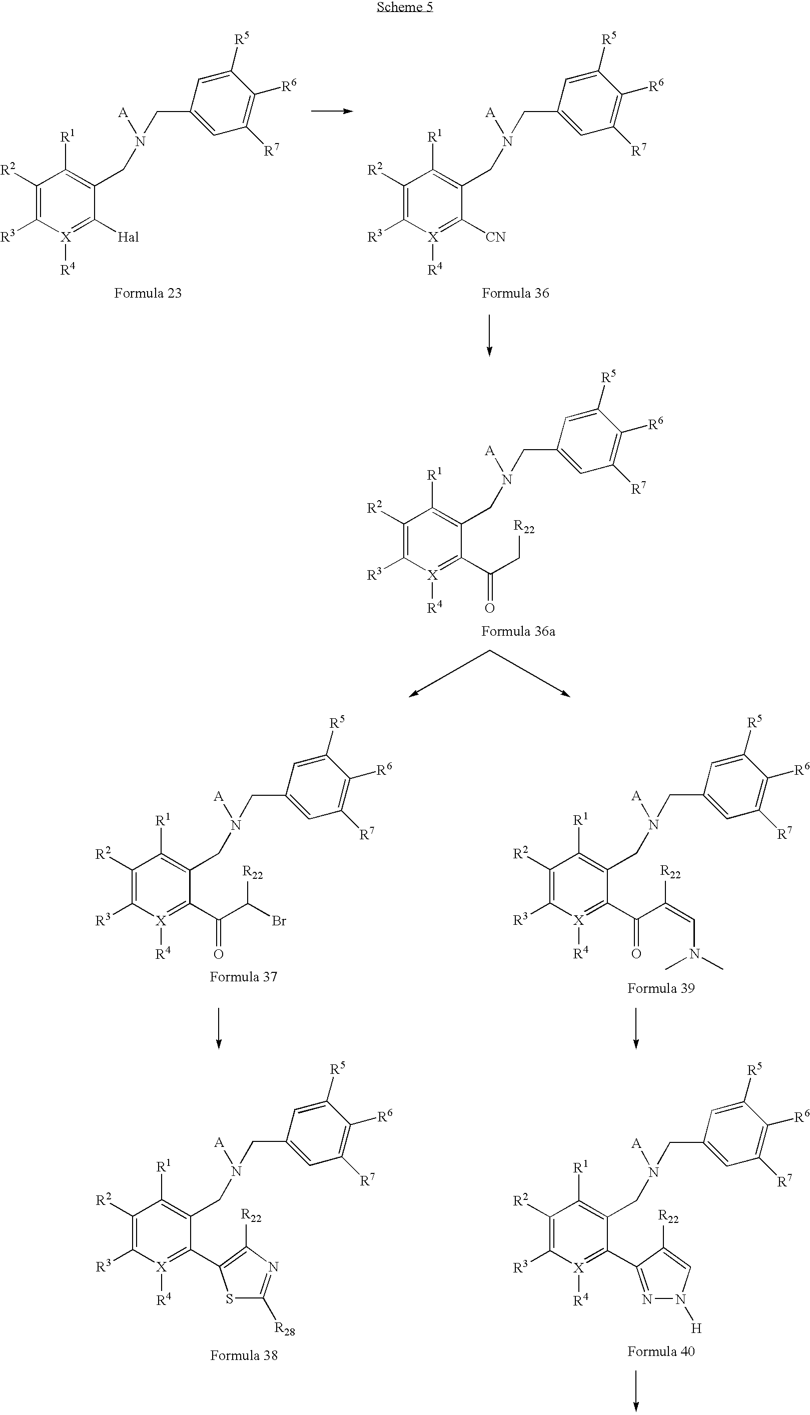

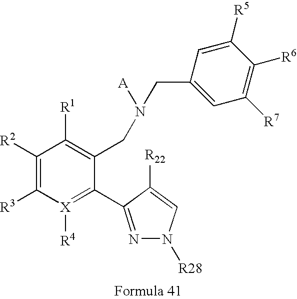

According to reaction Scheme 5, desired compounds of Formula 41, wherein A, X, R¹, R², R³, R⁴, R⁵, R⁶ and R⁷ are as described above, may be prepared by reacting compounds of Formula 23 and copper(l) cyanide in a suitable solvent such a dimethylformamide or N-methylpyrrolidinone to afford compounds of Formula 36. The solvent of choice is DMF at a temperature between 10° C. and 170° C., preferably 170° C.

The nitrile of Formula 36 may be converted into the ketone of Formula 36a, and subsequently to the ketone of Formula 39 by the addition of a Grignard reagent such as ethyl, n-propyl or butyl magnesium chloride in a suitable inert solvent such as THF or ether. In a microwave reactor, the preferred solvent is THF at a temperature between 40° C. and 60° C., preferably 60° C.

The corresponding ketone of Formula 39 may be treated with dimethylformamide-dimethylacetal (DMF-DMA) at a temperature between 40-110° C., preferably 110° C. for 1 to 12 hours to form compounds of Formula 40. Reacting compounds of Formula 40 with the addition alkyl or aryl hydrazine in a polar solvent such as methanol or ethanol at a temperature of 55-95° C., preferably 95° C., for 1 to 3.5 hours affords the compounds of Formula 41 where R²² and R²⁸ are optional substitutents of B as described herein.

Alternatively, the ketone of Formula 36a may be converted to the compounds of Formula 38 by addition of a solvent such as methylene chloride or chloroform to a solution of refluxing CuBr₂ in a solvent such as ethyl acetate for 1 to 6 hours, preferably 2 hours to afford the alpha bromo ketone of Formula 37. The alpha bromo ketone of Formula 37 is then dissolved in methanol or ethanol, preferably ethanol, and added to the corresponding thioacetamide. The reaction mixture may be heated to 50-90° C., preferably 90° C. for 6-12 hours, preferably 12 hours to afford the compounds of Formula 38 where R²² and R²⁸ are optional substitutents of B as described herein.

As an initial note, in the preparation of compounds, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, in the reaction schemes, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the compound.

Prodrugs of the compounds of the present invention may be prepared according to methods known to those skilled in the art. Exemplary processes are described below.

Prodrugs of this invention where a carboxyl group in a carboxylic acid of the compounds is replaced by an ester may be prepared by combining the carboxylic acid with the appropriate alkyl halide in the presence of a base such as potassium carbonate in an inert solvent such as dimethylformamide at a temperature of about 0 to 100° C. for about 1 to about 24 hours. Alternatively the acid is combined with an appropriate alcohol as solvent in the presence of a catalytic amount of acid such as concentrated sulfuric acid at a temperature of about 20 to 100° C., preferably at a reflux, for about 1 hour to about 24 hours. Another method is the reaction of the acid with a stoichiometric amount of the alcohol in the presence of a catalytic amount of acid in an inert solvent such as toluene or tetrahydrofuran, with concomitant removal of the water being produced by physical (e.g., Dean-Stark trap) or chemical (e.g., molecular sieves) means.

Prodrugs of this invention where an alcohol function has been derivatized as an ether may be prepared by combining the alcohol with the appropriate alkyl bromide or iodide in the presence of a base such as potassium carbonate in an inert solvent such as dimethylformamide at a temperature of about 0 to 100° C. for about 1 to about 24 hours. Alkanoylaminomethyl ethers may be obtained by reaction of the alcohol with a bis-alkanoylamino)methane in the presence of a catalytic amount of acid in an inert solvent such as tetrahydrofuran, according to a method described in U.S. Pat. No. 4,997,984. Alternatively, these compounds may be prepared by the methods described by Hoffman et al. in J. Org. Chem. 1994, 59, 3530.

Glycosides are prepared by reaction of the alcohol and a carbohydrate in an inert solvent such as toluene in the presence of acid. Typically the water formed in the reaction is removed as it is being formed as described above. An alternate procedure is the reaction of the alcohol with a suitably protected glycosyl halide in the presence of base followed by deprotection.

N-(1-hydroxyalkyl)amides, N-(1-hydroxy-1-(alkoxycarbonyl)methyl)amides may be prepared by the reaction of the parent amide with the appropriate aldehyde under neutral or basic conditions (e.g., sodium ethoxide in ethanol) at temperatures between 25 and 70° C. N-alkoxymethyl or N-1-(alkoxy)alkyl derivatives can be obtained by reaction of the N-unsubstituted compound with the necessary alkyl halide in the presence of a base in an inert solvent.

The compounds of this invention may also be used in conjunction with other pharmaceutical agents (e.g., LDL-cholesterol lowering agents, triglyceride lowering agents) for the treatment of the disease/conditions described herein. For example, they may be used in combination with a HMG-CoA reductase inhibitor, a cholesterol synthesis inhibitor, a cholesterol absorption inhibitor, another CETP inhibitor, a MTP/Apo B secretion inhibitor, a PPAR modulator and other cholesterol lowering agents such as a fibrate, niacin, an ion-exchange resin, an antioxidant, an ACAT inhibitor, and a bile acid sequestrant. Other pharmaceutical agents would also include the following: a bile acid reuptake inhibitor, an ileal bile acid transporter inhibitor, an ACC inhibitor, an antihypertensive (such as NORVASC®), a selective estrogen receptor modulator, a selective androgen receptor modulator, an antibiotic, an antidiabetic (such as metformin, a PPARγ activator, a sulfonylurea, insulin, an aldose reductase inhibitor (ARI) and a sorbitol dehydrogenase inhibitor (SDI)), and aspirin (acetylsalicylic acid or a nitric oxide releasing asprin). A slow-release form of niacin is available and is known as Niaspan. Niacin may also be combined with other therapeutic agents such as statins, i.e. lovastatin, which is an HMG-CoA reductase inhibitor and described further below. This combination therapy is known as ADVICOR® (Kos Pharmaceuticals Inc.) In combination therapy treatment, both the compounds of this invention and the other drug therapies are administered to mammals (e.g., humans, male or female) by conventional methods.

Any HMG-CoA reductase inhibitor may be used in the combination aspect of this invention. The term HMG-CoA reductase inhibitor refers to compounds which inhibit the bioconversion of hydroxymethylglutaryl-coenzyme A to mevalonic acid catalyzed by the enzyme HMG-CoA reductase. Such inhibition is readily determined by those skilled in the art according to standard assays (e.g., Meth. Enzymol. 1981; 71:455-509 and references cited therein). A variety of these compounds are described and referenced below however other HMG-CoA reductase inhibitors will be known to those skilled in the art. U.S. Pat. No. 4,231,938 (the disclosure of which is hereby incorporated by reference) discloses certain compounds isolated after cultivation of a microorganism belonging to the genus Aspergillus, such as lovastatin. Also, U.S. Pat. No. 4,444,784 (the disclosure of which is hereby incorporated by reference) discloses synthetic derivatives of the aforementioned compounds, such as simvastatin. Also, U.S. Pat. No. 4,739,073 (the disclosure of which is incorporated by reference) discloses certain substituted indoles, such as fluvastatin. Also, U.S. Pat. No. 4,346,227 (the disclosure of which is incorporated by reference) discloses ML-236B derivatives, such as pravastatin. Also, EP-491226A (the disclosure of which is incorporated by reference) discloses certain pyridyldihydroxyheptenoic acids, such as cerivastatin. In addition, U.S. Pat. No. 5,273,995 (the disclosure of which is incorporated by reference) discloses certain 6-[2-(substituted-pyrrol-1-yl)alkyl]pyran-2-ones such as atorvastatin and any pharmaceutically acceptable form thereof (i.e. LIPITOR®). Additional HMG-CoA reductase inhibitors include rosuvastatin and pitavastatin. Statins also include such compounds as rosuvastatin disclosed in U.S. RE37,314 E, pitavastatin disclosed in EP 304063 B1 and U.S. Pat. No. 5,011,930; mevastatin, disclosed in U.S. Pat. No. 3,983,140, which is incorporated herein by reference; velostatin, disclosed in U.S. Pat. No. 4,448,784 and U.S. Pat. No. 4,450,171, both of which are incorporated herein by reference; compactin, disclosed in U.S. Pat. No. 4,804,770, which is incorporated herein by reference; dalvastatin, disclosed in European Patent Application Publication No. 738510 A2; fluindostatin, disclosed in European Patent Application Publication No. 363934 A1; and dihydrocompactin, disclosed in U.S. Pat. No. 4,450,171, which is incorporated herein by reference.

Any PPAR modulator may be used in the combination aspect of this invention. The term PPAR modulator refers to compounds which modulate peroxisome proliferator activator receptor (PPAR) activity in mammals, particularly humans. Such modulation is readily determined by those skilled in the art according to standard assays known in the literature. It is believed that such compounds, by modulating the PPAR receptor, regulate transcription of key genes involved in lipid and glucose metabolism such as those in fatty acid oxidation and also those involved in high density lipoprotein (HDL) assembly (for example, apolipoprotein Al gene transcription), accordingly reducing whole body fat and increasing HDL cholesterol. By virtue of their activity, these compounds also reduce plasma levels of triglycerides, VLDL cholesterol, LDL cholesterol and their associated components such as apolipoprotein B in mammals, particularly humans, as well as increasing HDL cholesterol and apolipoprotein Al. Hence, these compounds are useful for the treatment and correction of the various dyslipidemias observed to be associated with the development and incidence of atherosclerosis and cardiovascular disease, including hypoalphalipoproteinemia and hypertriglyceridemia. A variety of these compounds are described and referenced below, however, others will be known to those skilled in the art. International Publication Nos. WO 02/064549 and 02/064130 and U.S. patent application Ser. No. 10/720,942, filed Nov. 24, 2003, U.S. patent application 60/552,114 filed Mar. 10, 2004 and U.S. patent application 60/583,721 filed Jun. 29, 2004 (the disclosures of which are hereby incorporated by reference) disclose certain compounds which are PPARα activators.

Any other PPAR modulator may be used in the combination aspect of this invention. In particular, modulators of PPARβ and/or PPARγ may be useful in combination with compounds of the present invention. An example PPAR inhibitor is described in US2003/0225158 as {5-Methoxy-2-methyl-4-[4-(4-trifluoromethyl-benzyloxy)-benzylsulfany]-phenoxy}-acetic acid.

Any MTP/Apo B (microsomal triglyceride transfer protein and or apolipoprotein B) secretion inhibitor may be used in the combination aspect of this invention. The term MTP/Apo B secretion inhibitor refers to compounds which inhibit the secretion of triglycerides, cholesteryl ester, and phospholipids. Such inhibition is readily determined by those skilled in the art according to standard assays (e.g., Wetterau, J. R. 1992; Science 258:999). A variety of these compounds are described and referenced below however other MTP/Apo B secretion inhibitors will be known to those skilled in the art, including imputapride (Bayer) and additional compounds such as those disclosed in WO 96/40640 and WO 98/23593, (two exemplary publications).

For example, the following MTP/Apo B secretion inhibitors are particularly useful:
4′-trifluoromethyl-biphenyl-2-carboxylic acid [2-(1H-[1,2,4]triazol-3-ylmethyl)-1,2,3,4-tetrahydro-isoquinolin-6-yl]-amide;
4′-trifluoromethyl-biphenyl-2-carboxylic acid [2-(2-acetylamino-ethyl)-1,2,3,4-tetrahydro-isoquinolin-6-yl]-amide;
(2-{6-[(4′-trifluoromethyl-biphenyl-2-carbonyl)-amino]-3,4-dihydro-1H-isoquinolin-2-yl}-ethyl)-carbamic acid methyl ester;
4′-trifluoromethyl-biphenyl-2-carboxylic acid [2-(1H-imidazol-2-ylmethyl)-1,2,3,4-tetrahydro-isoquinolin-6-yl]-amide;
4′-trifluoromethyl-biphenyl-2-carboxylic acid [2-(2,2-diphenyl-ethyl)-1,2,3,4-tetrahydro-isoquinolin-6-yl]-amide;
4′-trifluoromethyl-biphenyl-2-carboxylic acid [2-(2-ethoxy-ethyl)-1,2,3,4-tetrahydro-isoquinolin-6-yl]-amide;
(S)—N-{2-[benzyl(methyl)amino]-2-oxo-1-phenylethyl}-1-methyl-5-[4′-(trifluoromethyl)[1,1′-biphenyl]-2-carboxamido]-1H-indole-2-carboxamide;
(S)-2-[(4′-trifluoromethyl-biphenyl-2-carbonyl)-amino]-quinoline-6-carboxylic acid (pentylcarbamoyl-phenyl-methyl)amide;
1H-indole-2-carboxamide, 1-methyl-N-[(1S)-2-[methyl(phenylmethyl)amino]-2-oxo-1-phenylethyl]-5-[[[4′-(trifluoromethyl)[1,1′-biphenyl]-2-yl]carbonyl]amino]; and
N-[(1s)-2-(benzylmethylamino)-2-oxo-1-phenylethyl]-1-methyl-5-[[[4′-(trifluoromethyl)biphenyl-2-yl]carbonyl]amino]-1H-indole-2-carboxamide.

Any HMG-CoA synthase inhibitor may be used in the combination aspect of this invention. The term HMG-CoA synthase inhibitor refers to compounds which inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by those skilled in the art according to standard assays (Meth Enzymol. 1975; 35:155-160: Meth. Enzymol. 1985; 110:19-26 and references cited therein). A variety of these compounds are described and referenced below, however other HMG-CoA synthase inhibitors will be known to those skilled in the art. U.S. Pat. No. 5,120,729 (the disclosure of which is hereby incorporated by reference) discloses certain beta-lactam derivatives. U.S. Pat. No. 5,064,856 (the disclosure of which is hereby incorporated by reference) discloses certain spiro-lactone derivatives prepared by culturing a microorganism (MF5253). U.S. Pat. No. 4,847,271 (the disclosure of which is hereby incorporated by reference) discloses certain oxetane compounds such as 11-(3-hydroxymethyl-4-oxo-2-oxetayl)-3,5,7-trimethyl-2,4-undeca-dienoic acid derivatives.

Any compound that decreases HMG-CoA reductase gene expression may be used in the combination aspect of this invention. These agents may be HMG-CoA reductase transcription inhibitors that block the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such compounds may cause this effect by decreasing levels of SREBP (sterol receptor binding protein) by inhibiting the activity of site-1 protease (SI P) or agonizing the oxzgenal receptor or SCAP. Such regulation is readily determined by those skilled in the art according to standard assays (Meth. Enzymol. 1985; 110:9-19). Several compounds are described and referenced below, however other inhibitors of HMG-CoA reductase gene expression will be known to those skilled in the art. U.S. Pat. No. 5,041,432 (the disclosure of which is incorporated by reference) discloses certain 15-substituted lanosterol derivatives. Other oxygenated sterols that suppress synthesis of HMG-CoA reductase are discussed by E. I. Mercer (Prog. Lip. Res. 1993; 32:357-416).

Any additional compound having activity as a CETP inhibitor can serve as the second compound in the combination therapy aspect of the present invention. The term CETP inhibitor refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art, for example, those disclosed in commonly assigned U.S. Pat. No. 6,140,343 and commonly assigned U.S. Pat. No. 6,197,786. CETP inhibitors disclosed in these patents include compounds, such as [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, which is also known as torcetrapib. CETP inhibitors are also described in U.S. Pat. No. 6,723,752, which includes a number of CETP inhibitors including (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol. Moreover, CETP inhibitors included herein are also described in U.S. patent application Ser. No. 10/807,838 filed Mar. 23, 2004. U.S. Pat. No. 5,512,548 discloses certain polypeptide derivatives having activity as CETP inhibitors, while certain CETP-inhibitory rosenonolactone derivatives and phosphate-containing analogs of cholesteryl ester are disclosed in J. Antibiot., 49 (8): 815-816 (1996), and Bioorg. Med. Chem. Lett.; 6:1951-1954 (1996), respectively.

Any squalene synthetase inhibitor may be used in the combination aspect of this invention. The term squalene synthetase inhibitor refers to compounds which inhibit the condensation of 2 molecules of farnesylpyrophosphate to form squalene, catalyzed by the enzyme squalene synthetase. Such inhibition is readily determined by those skilled in the art according to standard assays (Meth. Enzymol. 1969; 15: 393-454 and Meth. Enzymol. 1985; 110:359-373 and references contained therein). A variety of these compounds are described in and referenced below however other squalene synthetase inhibitors will be known to those skilled in the art. U.S. Pat. No. 5,026,554 (the disclosure of which is incorporated by reference) discloses fermentation products of the microorganism MF5465 (ATCC 74011) including zaragozic acid. A summary of other patented squalene synthetase inhibitors has been compiled (Curr. Op. Ther. Patents (1993) 861-4).

Any squalene epoxidase inhibitor may be used in the combination aspect of this invention. The term squalene epoxidase inhibitor refers to compounds which inhibit the bioconversion of squalene and molecular oxygen into squalene-2,3-epoxide, catalyzed by the enzyme squalene epoxidase. Such inhibition is readily determined by those skilled in the art according to standard assays (Biochim. Biophys. Acta 1984; 794:466-471). A variety of these compounds are described and referenced below, however other squalene epoxidase inhibitors will be known to those skilled in the art. U.S. Pat. Nos. 5,011,859 and 5,064,864 (the disclosures of which are incorporated by reference) disclose certain fluoro analogs of squalene. EP publication 395,768 A (the disclosure of which is incorporated by reference) discloses certain substituted allylamine derivatives. PCT publication WO 9312069 A (the disclosure of which is hereby incorporated by reference) discloses certain amino alcohol derivatives. U.S. Pat. No. 5,051,534 (the disclosure of which is hereby incorporated by reference) discloses certain cyclopropyloxy-squalene derivatives.

Any squalene cyclase inhibitor may be used as the second component in the combination aspect of this invention. The term squalene cyclase inhibitor refers to compounds which inhibit the bioconversion of squalene-2,3-epoxide to lanosterol, catalyzed by the enzyme squalene cyclase. Such inhibition is readily determined by those skilled in the art according to standard assays (FEBS Lett. 1989; 244:347-350.). In addition, the compounds described and referenced below are squalene cyclase inhibitors, however other squalene cyclase inhibitors will also be known to those skilled in the art. PCT publication WO9410150 (the disclosure of which is hereby incorporated by reference) discloses certain 1,2,3,5,6,7,8,8a-octahydro-5,5,8(beta)-trimethyl-6-isoquinolineamine derivatives, such as N-trifluoroacetyl-1,2,3,5,6,7,8,8a-octahydro-2-allyl-5,5,8(beta)trimethyl-6(beta)-isoquinolineamine. French patent publication 2697250 (the disclosure of which is hereby incorporated by reference) discloses certain beta, beta-dimethyl-4-piperidine ethanol derivatives such as 1-(1,5,9-trimethyldecyl)-beta, beta-dimethyl-4-piperidineethanol

Any combined squalene epoxidase/squalene cyclase inhibitor may be used as the second component in the combination aspect of this invention. The term combined squalene epoxidase/squalene cyclase inhibitor refers to compounds that inhibit the bioconversion of squalene to lanosterol via a squalene-2,3-epoxide intermediate. In some assays it is not possible to distinguish between squalene epoxidase inhibitors and squalene cyclase inhibitors, however, these assays are recognized by those skilled in the art. Thus, inhibition by combined squalene epoxidase/squalene cyclase inhibitors is readily determined by those skilled in art according to the aforementioned standard assays for squalene cyclase or squalene epoxidase inhibitors. A variety of these compounds are described and referenced below, however other squalene epoxidase/squalene cyclase inhibitors will be known to those skilled in the art. U.S. Pat. Nos. 5,084,461 and 5,278,171 (the disclosures of which are incorporated by reference) disclose certain azadecalin derivatives. EP publication 468,434 (the disclosure of which is incorporated by reference) discloses certain piperidyl ether and thio-ether derivatives such as 2-(1-piperidyl)pentyl isopentyl sulfoxide and 2-(1-piperidyl)ethyl sulfide. PCT publication WO 9401404 (the disclosure of which is hereby incorporated by reference) discloses certain acyl-piperidines such as 1-(1-oxopentyl-5-phenylthio)-4-(2-hydroxy-1-methyl)ethyl)piperidine. U.S. Pat. No. 5,102,915 (the disclosure of which is hereby incorporated by reference) discloses certain cyclopropyloxy-squalene derivatives.

The compounds of the present invention can also be administered in combination with naturally occurring compounds that act to lower plasma cholesterol levels. These naturally occurring compounds are commonly called nutraceuticals and include, for example, garlic extract and niacin. A slow-release form of niacin is available and is known as Niaspan. Niacin may also be combined with other therapeutic agents such as lovastatin, or another is an HMG-CoA reductase inhibitor. This combination therapy with lovastatin is known as ADVICOR™ (Kos Pharmaceuticals Inc.).

Any cholesterol absorption inhibitor can be used as an additional in the combination aspect of the present invention. The term cholesterol absorption inhibition refers to the ability of a compound to prevent cholesterol contained within the lumen of the intestine from entering into the intestinal cells and/or passing from within the intestinal cells into the lymph system and/or into the blood stream. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480. An example of a recently approved cholesterol absorption inhibitor is ZETIA™ (ezetimibe) (Schering-Plough/Merck).

Any ACAT inhibitor may be used in the combination therapy aspect of the present invention. The term ACAT inhibitor refers to compounds that inhibit the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research., 24:1127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity. Examples of ACAT inhibitors include compounds such as Avasimibe (Pfizer), CS-505 (Sankyo) and Eflucimibe (Eli Lilly and Pierre Fabre).

A lipase inhibitor may be used in the combination therapy aspect of the present invention. A lipase inhibitor is a compound that inhibits the metabolic cleavage of dietary triglycerides or plasma phospholipids into free fatty acids and the corresponding glycerides (e.g. El, hi, etc.). Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a glyceride and fatty acid. In the intestine, the resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol. 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92,125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art. Preferred lipase inhibitors are those inhibitors that are selected from the group consisting of lipstatin, tetrahydrolipstatin (orlistat), valilactone, esterastin, ebelactone A, and ebelactone B. The compound tetrahydrolipstatin is especially preferred. The lipase inhibitor, N-3-trifluoromethylphenyl-N′-3-chloro-4′-trifluoromethylphenylurea, and the various urea derivatives related thereto, are disclosed in U.S. Pat. No. 4,405,644. The lipase inhibitor, esteracin, is disclosed in U.S. Pat. Nos. 4,189,438 and 4,242,453. The lipase inhibitor, cyclo-O, O′-[(1,6-hexanediyl)-bis-(iminocarbonyl)]dioxime, and the various bis(iminocarbonyl)dioximes related thereto may be prepared as described in Petersen et al., Liebig's Annalen, 562, 205-229 (1949).

A variety of pancreatic lipase inhibitors are described herein below. The pancreatic lipase inhibitors lipstatin, (2S,3S,5S,7Z,10Z)₅-[(S)-2-formamido-4-methyl-valeryloxy]-2-hexyl-3-hydroxy-7,10-hexadecanoic acid lactone, and tetrahydrolipstatin (orlistat), (2S,3S,5S)-5-[S)-2-formamido-4-methyl-valeryloxy]-2-hexyl-3-hydroxy-hexadecanoic 1,3 acid lactone, and the variously substituted N-formylleucine derivatives and stereoisomers thereof, are disclosed in U.S. Pat. No. 4,598,089. For example, tetrahydrolipstatin is prepared as described in, e.g., U.S. Pat. Nos. 5,274,143; 5,420,305; 5,540,917; and 5,643,874. The pancreatic lipase inhibitor, FL-386, 1-[4-(2-methylpropyl)cyclohexyl]-2-[(phenylsulfonyl)oxy]-ethanone, and the variously substituted sulfonate derivatives related thereto, are disclosed in U.S. Pat. No. 4,452,813. The pancreatic lipase inhibitor, WAY-121898, 4-phenoxyphenyl-4-methylpiperidin-1-yl-carboxylate, and the various carbamate esters and pharmaceutically acceptable salts related thereto, are disclosed in U.S. Pat. Nos. 5,512,565; 5,391,571 and 5,602,151. The pancreatic lipase inhibitor, valilactone, and a process for the preparation thereof by the microbial cultivation of Actinomycetes strain MG147-CF₂, are disclosed in Kitahara, et al., J. Antibiotics, 40 (11), 1647-1650 (1987). The pancreatic lipase inhibitors, ebelactone A and ebelactone B, and a process for the preparation thereof by the microbial cultivation of Actinomycetes strain MG7-G1, are disclosed in Umezawa, et al., J. Antibiotics, 33, 1594-1596 (1980). The use of ebelactones A and B in the suppression of monoglyceride formation is disclosed in Japanese Kokal 08-143457, published Jun. 4, 1996.

Other compounds that are marketed for hyperlipidemia, including hypercholesterolemia and which are intended to help prevent or treat atherosclerosis include bile acid sequestrants, such as Welchol®, Colestid®, LoCholest® and Questran®; and fibric acid derivatives, such as Atromid®, Lopid® and Tricor®.

Diabetes can be treated by administering to a patient having diabetes (especially Type II), insulin resistance, impaired glucose tolerance, metabolic syndrome, or the like, or any of the diabetic complications such as neuropathy, nephropathy, retinopathy or cataracts, a therapeutically effective amount of a compound of the present invention in combination with other agents (e.g., insulin) that can be used to treat diabetes. This includes the classes of anti-diabetic agents (and specific agents) described herein.

Any glycogen phosphorylase inhibitor can be used as the second agent in combination with a compound of the present invention. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays (e.g., J. Med. Chem. 41 (1998) 2934-2938). A variety of glycogen phosphorylase inhibitors are known to those skilled in the art including those described in WO 96/39384 and WO 96/39385.

Any aldose reductase inhibitor can be used in combination with a compound of the present invention. The term aldose reductase inhibitor refers to compounds that inhibit the bioconversion of glucose to sorbitol, which is catalyzed by the enzyme aldose reductase. Aldose reductase inhibition is readily determined by those skilled in the art according to standard assays (e.g., J. Malone, Diabetes, 29:861-864 (1980). “Red Cell Sorbitol, an indicator of Diabetic Control”). A variety of aldose reductase inhibitors are known to those skilled in the art.

Any sorbitol dehydrogenase inhibitor can be used in combination with a compound of the present invention. The term sorbitol dehydrogenase inhibitor refers to compounds that inhibit the bioconversion of sorbitol to fructose which is catalyzed by the enzyme sorbitol dehydrogenase. Such sorbitol dehydrogenase inhibitor activity is readily determined by those skilled in the art according to standard assays (e.g., Analyt. Biochem (2000) 280: 329-331). A variety of sorbitol dehydrogenase inhibitors are known, for example, U.S. Pat. Nos. 5,728,704 and 5,866,578 disclose compounds and a method for treating or preventing diabetic complications by inhibiting the enzyme sorbitol dehydrogenase.

Any glucosidase inhibitor can be used in combination with a compound of the present invention. A glucosidase inhibitor inhibits the enzymatic hydrolysis of complex carbohydrates by glycoside hydrolases, for example amylase or maltase, into bioavailable simple sugars, for example, glucose. The rapid metabolic action of glucosidases, particularly following the intake of high levels of carbohydrates, results in a state of alimentary hyperglycemia which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known chyme remaining in the stomach promotes the production of gastric juice, which initiates or favors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969) 8: 4214).

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethyl)-D-epi-inositol, and the various N-substituted pseudo-aminosugars related thereto, are disclosed in U.S. Pat. No. 4,701,559. The glucosidase inhibitor, miglitol, (2R,3R,4R,5S)-1-(2-hydroxyethyl)-2-(hydroxymethyl)-3,4,5-piperidinetriol, and the various 3,4,5-trihydroxypiperidines related thereto, are disclosed in U.S. Pat. No. 4,639,436. The glucosidase inhibitor, emiglitate, ethyl p-[2-[(2R,3R,4R,5S)-3,4,5-trihydroxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-O-β-D-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-[(2R,3R,4R,5S-3,4,5-trihydroxy-2-(hydroxymethyl)piperidino]-α-D-glucopyranoside sesquihydrate, the deoxy-nojirimycin derivatives related thereto, the various pharmaceutically acceptable salts thereof and synthetic methods for the preparation thereof, are disclosed in U.S. Pat. Nos. 5,157,116 and 5,504,078. The glycosidase inhibitor, salbostatin and the various pseudosaccharides related thereto, are disclosed in U.S. Pat. No. 5,091,524.

A variety of amylase inhibitors are known to one of ordinary skill in the art. The amylase inhibitor, tendamistat and the various cyclic peptides related thereto, are disclosed in U.S. Pat. No. 4,451,455. The amylase inhibitor Al-3688 and the various cyclic polypeptides related thereto are disclosed in U.S. Pat. No. 4,623,714. The amylase inhibitor, trestatin, consisting of a mixture of trestatin A, trestatin B and trestatin C and the various trehalose-containing aminosugars related thereto are disclosed in U.S. Pat. No. 4,273,765.

Additional anti-diabetic compounds, which can be used as the second agent in combination with a compound of the present invention, include, for example, the following: biguanides (e.g., metformin), insulin secretagogues (e.g., sulfonylureas and glinides), glitazones, non-glitazone PPARγ agonists, PPARβ agonists, inhibitors of DPP-IV, inhibitors of PDE5, inhibitors of GSK-3, glucagon antagonists, inhibitors of f-1,6-BPase (Metabasis/Sankyo), GLP-1/analogs (AC 2993, also known as exendin-4), insulin and insulin mimetics (Merck natural products). Other examples would include PKC-β inhibitors and AGE breakers.

The compounds of the present invention can be used in combination with anti-obesity agents. Any anti-obesity agent can be used as the second agent in such combinations and examples are provided herein. Such anti-obesity activity is readily determined by those skilled in the art according to standard assays known in the art.

Suitable anti-obesity agents include phenylpropanolamine, ephedrine, pseudoephedrine, phentermine, β₃ adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine), sympathomimetic agents, serotoninergic agents, cannabinoid receptor (CB-1) antagonists (e.g., rimonabant described in U.S. Pat. No. 5,624,941 (SR-141,716A), purine compounds, such as those described in US Patent Publication No. 2004/0092520; pyrazolo[1,5-a][1,3,5]triazine compounds, such as those described in U.S. Non-Provisional patent application Ser. No. 10/763,105 filed on Jan. 21, 2004; and bicyclic pyrazolyl and imidazolyl compounds, such as those described in U.S. Provisional Application No. 60/518,280 filed on Nov. 7, 2003), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelin receptor antagonists, histamine 3 receptor antagonists or inverse agonists, neuromedin U receptor agonists, and the like.

Any thyromimetic can be used as the second agent in combination with a compound of the present invention. Such thyromimetic activity is readily determined by those skilled in the art according to standard assays (e.g., Atherosclerosis (1996) 126: 53-63). A variety of thyromimetic agents are known to those skilled in the art, for example those disclosed in U.S. Pat. Nos. 4,766,121; 4,826,876; 4,910,305; 5,061,798; 5,284,971; 5,401,772; 5,654,468; and 5,569,674. Other antiobesity agents include sibutramine which can be prepared as described in U.S. Pat. No. 4,929,629. and bromocriptine which can be prepared as described in U.S. Pat. Nos. 3,752,814 and 3,752,888.

The compounds of the present invention can also be used in combination with other antihypertensive agents. Any anti-hypertensive agent can be used as the second agent in such combinations and examples are provided herein. Such antihypertensive activity is readily determined by those skilled in the art according to standard assays (e.g., blood pressure measurements).

Examples of presently marketed products containing antihypertensive agents include calcium channel blockers, such as Cardizem®, Adalat®, Calan®, Cardene®, Covera®, Dilacor®, DynaCirc®, Procardia XL®, Sular®, Tiazac®, Vascor®, Verelan®, Isoptin®, Nimotop®, Norvasc®, and Plendil®; angiotensin converting enzyme (ACE) inhibitors, such as Accupril®, Altace®, Captopril®, Lotensin®, Mavik®, Monopril®, Prinivil®, Univasc®, Vasotec® and Zestril®.

Amlodipine and related dihydropyridine compounds are disclosed in U.S. Pat. No. 4,572,909, which is incorporated herein by reference, as potent anti-ischemic and antihypertensive agents. U.S. Pat. No. 4,879,303, which is incorporated herein by reference, discloses amlodipine benzenesulfonate salt (also termed amlodipine besylate). Amlodipine and amlodipine besylate are potent and long lasting calcium channel blockers. As such, amlodipine, amlodipine besylate, amlodipine maleate and other pharmaceutically acceptable acid addition salts of amlodipine have utility as antihypertensive agents and as antiischemic agents. Amlodipine besylate is currently sold as Norvasc®. Amlodipine has the formula

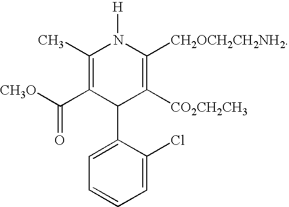

Calcium channel blockers which are within the scope of this invention include, but are not limited to: bepridil, which may be prepared as disclosed in U.S. Pat. No. 3,962,238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, which may be prepared as disclosed in U.S. Pat. No. 4,808,605; prenylamine, which may be prepared as disclosed in U.S. Pat. No. 3,152,173; semotiadil, which may be prepared as disclosed in U.S. Pat. No. 4,786,635; terodiline, which may be prepared as disclosed in U.S. Pat. No. 3,371,014; verapamil, which may be prepared as disclosed in U.S. Pat. No. 3,261,859; aranipine, which may be prepared as disclosed in U.S. Pat. No. 4,572,909; bamidipine, which may be prepared as disclosed in U.S. Pat. No. 4,220,649; benidipine, which may be prepared as disclosed in European Patent Application Publication No. 106,275; cilnidipine, which may be prepared as disclosed in U.S. Pat. No. 4,672,068; efonidipine, which may be prepared as disclosed in U.S. Pat. No. 4,885,284; elgodipine, which may be prepared as disclosed in U.S. Pat. No. 4,952,592; felodipine, which may be prepared as disclosed in U.S. Pat. No. 4,264,611; isradipine, which may be prepared as disclosed in U.S. Pat. No. 4,466,972; lacidipine, which may be prepared as disclosed in U.S. Pat. No. 4,801,599; lercanidipine, which may be prepared as disclosed in U.S. Pat. No. 4,705,797; manidipine, which may be prepared as disclosed in U.S. Pat. No. 4,892,875; nicardipine, which may be prepared as disclosed in U.S. Pat. No. 3,985,758; nifedipine, which may be prepared as disclosed in U.S. Pat. No. 3,485,847; nilvadipine, which may be prepared as disclosed in U.S. Pat. No. 4,338,322; nimodipine, which may be prepared as disclosed in U.S. Pat. No. 3,799,934; nisoldipine, which may be prepared as disclosed in U.S. Pat. No. 4,154,839; nitrendipine, which may be prepared as disclosed in U.S. Pat. No. 3,799,934; cinnarizine, which may be prepared as disclosed in U.S. Pat. No. 2,882,271; flunarizine, which may be prepared as disclosed in U.S. Pat. No. 3,773,939; lidoflazine, which may be prepared as disclosed in U.S. Pat. No. 3,267,104; lomerizine, which may be prepared as disclosed in U.S. Pat. No. 4,663,325; bencyclane, which may be prepared as disclosed in Hungarian Patent No. 151,865; etafenone, which may be prepared as disclosed in German Patent No. 1,265,758; and perhexyline, which may be prepared as disclosed in British Patent No. 1,025,578. The disclosures of all such U.S. patents are incorporated herein by reference.

Angiotensin Converting Enzyme Inhibitors (ACE-inhibitors) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361. The disclosures of all such U.S. patents are incorporated herein by reference.

Angiotensin-II receptor antagonists (A-II antagonists) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; and valsartan, which may be prepared as disclosed in U.S. Pat. No. 5,399,578. The disclosures of all such U.S. patents are incorporated herein by reference.

Beta-adrenergic receptor blockers (beta- or β-blockers) which are within the scope of this invention include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. No. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may be prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071; buprandolol, which may be prepared as disclosed in U.S. Pat. No. 3,309,406; butiridine hydrochloride, which may be prepared as disclosed in French Patent No. 1,390,056; butofilolol, which may be prepared as disclosed in U.S. Pat. No. 4,252,825; carazolol, which may be prepared as disclosed in German Patent No. 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; cloranolol, which may be prepared as disclosed in German Patent No. 2,213,044; dilevalol, which may be prepared as disclosed in Clifton et al., Journal of Medicinal Chemistry, 1982, 25, 670; epanolol, which may be prepared as disclosed in European Patent Publication Application No. 41,491; indenolol, which may be prepared as disclosed in U.S. Pat. No. 4,045,482; labetalol, which may be prepared as disclosed in U.S. Pat. No. 4,012,444; levobunolol, which may be prepared as disclosed in U.S. Pat. No. 4,463,176; mepindolol, which may be prepared as disclosed in Seeman et al., Helv. Chim. Acta, 1971, 54, 241; metipranolol, which may be prepared as disclosed in Czechoslovakian Patent Application No. 128,471; metoprolol, which may be prepared as disclosed in U.S. Pat. No. 3,873,600; moprolol, which may be prepared as disclosed in U.S. Pat. No. 3,501,769I; nadolol, which may be prepared as disclosed in U.S. Pat. No. 3,935,267; nadoxolol, which may be prepared as disclosed in U.S. Pat. No. 3,819,702; nebivalol, which may be prepared as disclosed in U.S. Pat. No. 4,654,362; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,357; propranolol, which may be prepared as disclosed in U.S. Pat. Nos. 3,337,628 and 3,520,919; sotalol, which may be prepared as disclosed in Uloth et al., Journal of Medicinal Chemistry, 1966, 9, 88; sufinalol, which may be prepared as disclosed in German Patent No. 2,728,641; talindol, which may be prepared as disclosed in U.S. Pat. Nos. 3,935,259 and 4,038,313; tertatolol, which may be prepared as disclosed in U.S. Pat. No. 3,960,891; tillsolol, which may be prepared as disclosed in U.S. Pat. No. 4,129,565; timolol, which may be prepared as disclosed in U.S. Pat. No. 3,655,663; toliprolol, which may be prepared as disclosed in U.S. Pat. No. 3,432,545; and xibenolol, which may be prepared as disclosed in U.S. Pat. No. 4,018,824. The disclosures of all such U.S. patents are incorporated herein by reference.

Alpha-adrenergic receptor blockers (alpha- or α-blockers) which are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may be prepared as disclosed in U.S. Pat. No. 3,669,968; and yohimbine, which may be isolated from natural sources according to methods well known to those skilled in the art. The disclosures of all such U.S. patents are incorporated herein by reference.

The term ‘vasodilator,’ where used herein, is meant to include cerebral vasodilators, coronary vasodilators and peripheral vasodilators. Cerebral vasodilators within the scope of this invention include, but are not limited to: bencyclane; cinnarizine; citicoline, which may be isolated from natural sources as disclosed in Kennedy et al., Journal of the American Chemical Society, 1955, 77, 250 or synthesized as disclosed in Kennedy, Journal of Biological Chemistry, 1956, 222, 185; cyclandelate, which may be prepared as disclosed in U.S. Pat. No. 3,663,597; ciclonicate, which may be prepared as disclosed in German Patent No. 1,910,481; diisopropylamine dichloroacetate, which may be prepared as disclosed in British Patent No. 862,248; eburnamonine, which may be prepared as disclosed in Hermann et al., Journal of the American Chemical Society, 1979, 101, 1540; fasudil, which may be prepared as disclosed in U.S. Pat. No. 4,678,783; fenoxedil, which may be prepared as disclosed in U.S. Pat. No. 3,818,021; flunarizine, which may be prepared as disclosed in U.S. Pat. No. 3,773,939; ibudilast, which may be prepared as disclosed in U.S. Pat. No. 3,850,941; ifenprodil, which may be prepared as disclosed in U.S. Pat. No. 3,509,164; lomerizine, which may be prepared as disclosed in U.S. Pat. No. 4,663,325; nafronyl, which may be prepared as disclosed in U.S. Pat. No. 3,334,096; nicametate, which may be prepared as disclosed in Blicke et al., Journal of the American Chemical Society, 1942, 64, 1722; nicergoline, which may be prepared as disclosed above; nimodipine, which may be prepared as disclosed in U.S. Pat. No. 3,799,934; papaverine, which may be prepared as reviewed in Goldberg, Chem. Prod. Chem. News, 1954, 17 371; pentifylline, which may be prepared as disclosed in German Patent No. 860,217; tinofedrine, which may be prepared as disclosed in U.S. Pat. No. 3,563,997; vincamine, which may be prepared as disclosed in U.S. Pat. No. 3,770,724; vinpocetine, which may be prepared as disclosed in U.S. Pat. No. 4,035,750; and viquidil, which may be prepared as disclosed in U.S. Pat. No. 2,500,444. The disclosures of all such U.S. patents are incorporated herein by reference.

Coronary vasodilators within the scope of this invention include, but are not limited to: amotriphene, which may be prepared as disclosed in U.S. Pat. No. 3,010,965; bendazol, which may be prepared as disclosed in J. Chem. Soc. 1958, 2426; benfurodil hemisuccinate, which may be prepared as disclosed in U.S. Pat. No. 3,355,463; benziodarone, which may be prepared as disclosed in U.S. Pat. No. 3,012,042; chloracizine, which may be prepared as disclosed in British Patent No. 740,932; chromonar, which may be prepared as disclosed in U.S. Pat. No. 3,282,938; clobenfural, which may be prepared as disclosed in British Patent No. 1,160,925; clonitrate, which may be prepared from propanediol according to methods well known to those skilled in the art, e.g., see Annalen, 1870, 155, 165; cloricromen, which may be prepared as disclosed in U.S. Pat. No. 4,452,811; dilazep, which may be prepared as disclosed in U.S. Pat. No. 3,532,685; dipyridamole, which may be prepared as disclosed in British Patent No. 807,826; droprenilamine, which may be prepared as disclosed in German Patent No. 2,521,113; efloxate, which may be prepared as disclosed in British Patent Nos. 803,372 and 824,547; erythrityl tetranitrate, which may be prepared by nitration of erythritol according to methods well-known to those skilled in the art; etafenone, which may be prepared as disclosed in German Patent No. 1,265,758; fendiline, which may be prepared as disclosed in U.S. Pat. No. 3,262,977; floredil, which may be prepared as disclosed in German Patent No. 2,020,464; ganglefene, which may be prepared as disclosed in U.S.S.R. Patent No. 115,905; hexestrol, which may be prepared as disclosed in U.S. Pat. No. 2,357,985; hexobendine, which may be prepared as disclosed in U.S. Pat. No. 3,267,103; itramin tosylate, which may be prepared as disclosed in Swedish Patent No. 168,308; khellin, which may be prepared as disclosed in Baxter et al., Journal of the Chemical Society, 1949, S 30; lidoflazine, which may be prepared as disclosed in U.S. Pat. No. 3,267,104; mannitol hexanitrate, which may be prepared by the nitration of mannitol according to methods well-known to those skilled in the art; medibazine, which may be prepared as disclosed in U.S. Pat. No. 3,119,826; nitroglycerin; pentaerythritol tetranitrate, which may be prepared by the nitration of pentaerythritol according to methods well-known to those skilled in the art; pentrinitrol, which may be prepared as disclosed in German Patent No. 638, 422-3; perhexylline, which may be prepared as disclosed above; pimethylline, which may be prepared as disclosed in U.S. Pat. No. 3,350,400; prenylamine, which may be prepared as disclosed in U.S. Pat. No. 3,152,173; propatyl nitrate, which may be prepared as disclosed in French Patent No. 1,103,113; trapidil, which may be prepared as disclosed in East German Patent No. 55,956; tricromyl, which may be prepared as disclosed in U.S. Pat. No. 2,769,015; trimetazidine, which may be prepared as disclosed in U.S. Pat. No. 3,262,852; trolnitrate phosphate, which may be prepared by nitration of triethanolamine followed by precipitation with phosphoric acid according to methods well-known to those skilled in the art; visnadine, which may be prepared as disclosed in U.S. Pat. Nos. 2,816,118 and 2,980,699. The disclosures of all such U.S. patents are incorporated herein by reference.

Peripheral vasodilators within the scope of this invention include, but are not limited to: aluminum nicotinate, which may be prepared as disclosed in U.S. Pat. No. 2,970,082; bamethan, which may be prepared as disclosed in Corrigan et al., Journal of the American Chemical Society, 1945, 67, 1894; bencyclane, which may be prepared as disclosed above; betahistine, which may be prepared as disclosed in Walter et al.; Journal of the American Chemical Society, 1941, 63, 2771; bradykinin, which may be prepared as disclosed in Hamburg et al., Arch. Biochem. Biophys., 1958, 76, 252; brovincamine, which may be prepared as disclosed in U.S. Pat. No. 4,146,643; bufeniode, which may be prepared as disclosed in U.S. Pat. No. 3,542,870; buflomedil, which may be prepared as disclosed in U.S. Pat. No. 3,895,030; butalamine, which may be prepared as disclosed in U.S. Pat. No. 3,338,899; cetiedil, which may be prepared as disclosed in French Patent Nos. 1,460,571; ciclonicate, which may be prepared as disclosed in German Patent No. 1,910,481; cinepazide, which may be prepared as disclosed in Belgian Patent No. 730,345; cinnarizine, which may be prepared as disclosed above; cyclandelate, which may be prepared as disclosed above; diisopropylamine dichloroacetate, which may be prepared as disclosed above; eledoisin, which may be prepared as disclosed in British Patent No. 984,810; fenoxedll, which may be prepared as disclosed above; flunarizine, which may be prepared as disclosed above; hepronicate, which may be prepared as disclosed in U.S. Pat. No. 3,384,642; ifenprodil, which may be prepared as disclosed above; iloprost, which may be prepared as disclosed in U.S. Pat. No. 4,692,464; inositol niacinate, which may be prepared as disclosed in Badgett et al., Journal of the American Chemical Society, 1947, 69, 2907; isoxsuprine, which may be prepared as disclosed in U.S. Pat. No. 3,056,836; kallidin, which may be prepared as disclosed in Biochem. Biophys. Res. Commun., 1961, 6, 210; kallikrein, which may be prepared as disclosed in German Patent No. 1,102,973; moxisylyte, which may be prepared as disclosed in German Patent No. 905,738; nafronyl, which may be prepared as disclosed above; nicametate, which may be prepared as disclosed above; nicergoline, which may be prepared as disclosed above; nicofuranose, which may be prepared as disclosed in Swiss Patent No. 366,523; nylidrin, which may be prepared as disclosed in U.S. Pat. Nos. 2,661,372 and 2,661,373; pentifylline, which may be prepared as disclosed above; pentoxifylline, which may be prepared as disclosed in U.S. Pat. No. 3,422,107; piribedil, which may be prepared as disclosed in U.S. Pat. No. 3,299,067; prostaglandin E1, which may be prepared by any of the methods referenced in the Merck Index, Twelfth Edition, Budaveri, Ed., N.J., 1996, p. 1353; suloctidil, which may be prepared as disclosed in German Patent No. 2,334,404; tolazoline, which may be prepared as disclosed in U.S. Pat. No. 2,161,938; and xanthinol niacinate, which may be prepared as disclosed in German Patent No. 1,102,750 or Korbonits et al., Acta. Pharm. Hung., 1968, 38, 98. The disclosures of all such U.S. patents are incorporated herein by reference.

The term “diuretic,” within the scope of this invention, is meant to include diuretic benzothiadiazine derivatives, diuretic organomercurials, diuretic purines, diuretic steroids, diuretic sulfonamide derivatives, diuretic uracils and other diuretics such as amanozine, which may be prepared as disclosed in Austrian Patent No. 168,063; amiloride, which may be prepared as disclosed in Belgian Patent No. 639,386; arbutin, which may be prepared as disclosed in Tschitschibabin, Annalen, 1930, 479, 303; chlorazanil, which may be prepared as disclosed in Austrian Patent No. 168,063; ethacrynic acid, which may be prepared as disclosed in U.S. Pat. No. 3,255,241; etozolin, which may be prepared as disclosed in U.S. Pat. No. 3,072,653; hydracarbazine, which may be prepared as disclosed in British Patent No. 856,409; isosorbide, which may be prepared as disclosed in U.S. Pat. No. 3,160,641; mannitol; metochalcone, which may be prepared as disclosed in Freudenberg et al., Ber., 1957, 90, 957; muzolimine, which may be prepared as disclosed in U.S. Pat. No. 4,018,890; perhexyline, which may be prepared as disclosed above; ticrynafen, which may be prepared as disclosed in U.S. Pat. No. 3,758,506; triamterene which may be prepared as disclosed in U.S. Pat. No. 3,081,230; and urea. The disclosures of all such U.S. patents are incorporated herein by reference.

Diuretic benzothiadiazine derivatives within the scope of this invention include, but are not limited to: althiazide, which may be prepared as disclosed in British Patent No. 902,658; bendroflumethiazide, which may be prepared as disclosed in U.S. Pat. No. 3,265,573; benzthiazide, McManus et al., 136th Am. Soc. Meeting (Atlantic City, September 1959), Abstract of papers, pp 13-O; benzylhydrochlorothiazide, which may be prepared as disclosed in U.S. Pat. No. 3,108,097; buthiazide, which may be prepared as disclosed in British Patent Nos. 861,367 and 885,078; chlorothiazide, which may be prepared as disclosed in U.S. Pat. Nos. 2,809,194 and 2,937,169; chlorthalidone, which may be prepared as disclosed in U.S. Pat. No. 3,055,904; cyclopenthiazide, which may be prepared as disclosed in Belgian Patent No. 587,225; cyclothiazide, which may be prepared as disclosed in Whitehead et al., Journal of Organic Chemistry, 1961, 26, 2814; epithiazide, which may be prepared as disclosed in U.S. Pat. No. 3,009,911; ethiazide, which may be prepared as disclosed in British Patent No. 861,367; fenquizone, which may be prepared as disclosed in U.S. Pat. No. 3,870,720; indapamide, which may be prepared as disclosed in U.S. Pat. No. 3,565,911; hydrochlorothiazide, which may be prepared as disclosed in U.S. Pat. No. 3,164,588; hydroflumethiazide, which may be prepared as disclosed in U.S. Pat. No. 3,254,076; methyclothiazide, which may be prepared as disclosed in Close et al., Journal of the American Chemical Society, 1960, 82, 1132; meticrane, which may be prepared as disclosed in French Patent Nos. M2790 and 1,365,504; metolazone, which may be prepared as disclosed in U.S. Pat. No. 3,360,518; paraflutizide, which may be prepared as disclosed in Belgian Patent No. 620,829; polythiazide, which may be prepared as disclosed in U.S. Pat. No. 3,009,911; quinethazone, which may be prepared as disclosed in U.S. Pat. No. 2,976,289; teclothiazide, which may be prepared as disclosed in Close et al., Journal of the American Chemical Society, 1960, 82,1132; and trichlormethiazide, which may be prepared as disclosed in deStevens et al., Experientia, 1960, 16, 113. The disclosures of all such U.S. patents are incorporated herein by reference.

Diuretic sulfonamide derivatives within the scope of this invention include, but are not limited to: acetazolamide, which may be prepared as disclosed in U.S. Pat. No. 2,980,679; ambuside, which may be prepared as disclosed in U.S. Pat. No. 3,188,329; azosemide, which may be prepared as disclosed in U.S. Pat. No. 3,665,002; bumetanide, which may be prepared as disclosed in U.S. Pat. No. 3,634,583; butazolamide, which may be prepared as disclosed in British Patent No. 769,757; chloraminophenamide, which may be prepared as disclosed in U.S. Pat. Nos. 2,809,194, 2,965,655 and 2,965,656; clofenamide, which may be prepared as disclosed in Olivier, Rec. Trav. Chim., 1918, 37, 307; clopamide , which may be prepared as disclosed in U.S. Pat. No. 3,459,756; clorexolone, which may be prepared as disclosed in U.S. Pat. No. 3,183,243; disulfamide, which may be prepared as disclosed in British Patent No. 851,287; ethoxolamide, which may be prepared as disclosed in British Patent No. 795,174; furosemide, which may be prepared as disclosed in U.S. Pat. No. 3,058,882; mefruside, which may be prepared as disclosed in U.S. Pat. No. 3,356,692; methazolamide, which may be prepared as disclosed in U.S. Pat. No. 2,783,241; piretanide, which may be prepared as disclosed in U.S. Pat. No. 4,010,273; torasemide, which may be prepared as disclosed in U.S. Pat. No. 4,018,929; tripamide, which may be prepared as disclosed in Japanese Patent No. 73 05,585; and xipamide, which may be prepared as disclosed in U.S. Pat. No. 3,567,777. The disclosures of all such U.S. patents are incorporated herein by reference.

Osteoporosis is a systemic skeletal disease, characterized by low bone mass and deterioration of bone tissue, with a consequent increase in bone fragility and susceptibility to fracture. In the U.S., the condition affects more than 25 million people and causes more than 1.3 million fractures each year, including 500,000 spine, 250,000 hip and 240,000 wrist fractures annually. Hip fractures are the most serious consequence of osteoporosis, with 5-20% of patients dying within one year, and over 50% of survivors being incapacitated.

The elderly are at greatest risk of osteoporosis, and the problem is therefore predicted to increase significantly with the aging of the population. Worldwide fracture incidence is forecasted to increase three-fold over the next 60 years, and one study has estimated that there will be 4.5 million hip fractures worldwide in 2050.

Women are at greater risk of osteoporosis than men. Women experience a sharp acceleration of bone loss during the five years following menopause. Other factors that increase the risk include smoking, alcohol abuse, a sedentary lifestyle and low calcium intake.

Those skilled in the art will recognize that anti-resorptive agents (for example progestins, polyphosphonates, bisphosphonate(s), estrogen agonists/antagonists, estrogen, estrogen/progestin combinations, Premarin®, estrone, estriol or 17α- or 17β-ethynyl estradiol) may be used in conjunction with the compounds of the present invention.

Exemplary progesuns are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, fluorogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol.

Preferred progestins are medroxyprogestrone, norethindrone and norethynodrel.

Exemplary bone resorption inhibiting polyphosphonates include polyphosphonates of the type disclosed in U.S. Pat. No. 3,683,080, the disclosure of which is incorporated herein by reference. Preferred polyphosphonates are geminal diphosphonates (also referred to as bis-phosphonates). Tiludronate disodium is an especially preferred polyphosphonate. Ibandronic acid is an especially preferred polyphosphonate. Alendronate and resindronate are especially preferred polyphosphonates. Zoledronic acid is an especially preferred polyphosphonate. Other preferred polyphosphonates are 6-amino-1-hydroxy-hexylidene-bisphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid, N,N-dimethylamino methane diphosphonic acid, N(2-hydroxyethyl)amino methane diphosphonic acid, butane-4-amino-1-hydroxy-1,1-diphosphonic acid, pentane-5-amino-1-hydroxy-1,1-diphosphonic acid, hexane-6-amino-1-hydroxy-1,1-diphosphonic acid and pharmaceutically acceptable esters and salts thereof.

In particular, the compounds of this invention may be combined with a mammalian estrogen agonist/antagonist. Any estrogen agonist/antagonist may be used in the combination aspect of this invention. The term estrogen agonist/antagonist refers to compounds which bind with the estrogen receptor, inhibit bone turnover and/or prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue, and blocking the actions of estrogen in one or more tissues. Such activities are readily determined by those skilled in the art of standard assays including estrogen receptor binding assays, standard bone histomorphometric and densitometer methods, and Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31 (1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below.

Another preferred estrogen agonist/antagonist is 3-(4-(1,2-diphenyl-but-1-enyl)-phenyl)-acrylic acid, which is disclosed in Willson et al., Endocrinology, 1997, 138, 3901-3911.

Another preferred estrogen agonist/antagonist is tamoxifen: (ethanamine, 2-(-4-(1,2-diphenyl-1-butenyl)phenoxy)-N,N-dimethyl, (Z)-2-, 2-hydroxy-1,2,3-propanetricarboxylate(1:1)) and related compounds which are disclosed in U.S. Pat. No. 4,536,516, the disclosure of which is incorporated herein by reference.

Another related compound is 4-hydroxy tamoxifen, which is disclosed in U.S. Pat. No. 4,623,660, the disclosure of which is incorporated herein by reference.

A preferred estrogen agonist/antagonist is raloxifene: (methanone, (6-hydroxy-2-(4-hydroxyphenyl)benzo[b]thien-3-yl)(4-(2-(1-piperidinyl)ethoxy)phenyl)-hydrochloride) which is disclosed in U.S. Pat. No. 4,418,068, the disclosure of which is incorporated herein by reference.

Another preferred estrogen agonist/antagonist is toremifene: (ethanamine, 2-(4-(4-chloro-1,2-diphenyl-1-butenyl)phenoxy)-N,N-dimethyl-, (Z)-, 2-hydroxy-1,2,3-propanetricarboxylate (1:1) which is disclosed in U.S. Pat. No. 4,996,225, the disclosure of which is incorporated herein by reference.

Another preferred estrogen agonist/antagonist is centchroman: 1-(2-((4-(-methoxy-2,2, dimethyl-3-phenyl-chroman-4-yl)-phenoxy)-ethylypyrrolidine, which is disclosed in U.S. Pat. No. 3,822,287, the disclosure of which is incorporated herein by reference. Also preferred is levormeloxifene.

Another preferred estrogen agonist/antagonist is idoxifene: (E)-1-(2-(4-(1-(4-iodo-phenyl)-2-phenyl-but-1-enylfiphenoxy)-ethylypyrrolidinone, which is disclosed in U.S. Pat. No. 4,839,155, the disclosure of which is incorporated herein by reference.

Another preferred estrogen agonist/antagonist is 2-(4-methoxy-phenyl)-3-[4-(2-piperidin-1-yl-ethoxy)-phenoxy]-benzo[b]thiophen-6-ol which is disclosed in U.S. Pat. No. 5,488,058, the disclosure of which is incorporated herein by reference.

Another preferred estrogen agonist/antagonist is 6-(4-hydroxy-phenyl)-5-(4-(2-piperidin-1-yl-ethoxy)benzyl)-naphthalen-2-ol, which is disclosed in U.S. Pat. No. 5,484,795, the disclosure of which is incorporated herein by reference.

Another preferred estrogen agonist/antagonist is (4-(2-(2-aza-bicyclo[2.2.1]hept-2-yl)ethoxy)-phenyl)-(6-hydroxy-2-(4-hydroxy-phenyl)-benzo[b]thiophen-3-yl)-methanone which is disclosed, along with methods of preparation, in PCT publication no. WO 95/10513 assigned to Pfizer inc.

Other preferred estrogen agonist/antagonists include the compounds, TSE-424 (Wyeth-Ayerst Laboratories) and arazoxifene.

Other preferred estrogen agonist/antagonists include compounds as described in commonly assigned U.S. Pat. No. 5,552,412, the disclosure of which is incorporated herein by reference. Especially preferred compounds described therein are:
cis-6-(4-fluoro-phenyl)-5-(4-(2-piperidin-1-yl-ethoxy)-phenyl)-5,6,7,8-tetrahydro-naphthalene-2-ol;
(−)-cis-6-phenyl-5-(4-(2-pyrrolidin-1-yl-ethoxy)-phenyl)-5,6,7,8-tetrahydro-naphthalene-2-ol (also known as lasofoxifene);
cis-6-phenyl-5-(4-(2-pyrrolidin-1-yl-ethoxy)-phenyl)-5,6,7,8-tetrahydro-naphthalene-2-ol;
cis-1-(6′-pyrrolodinoethoxy-3′-pyridyl)-2-phenyl-6-hydroxy-1,2,3,4-tetrahydronaphthalene;
1-(4′-pyrrolidinoethoxyphenyl)-2-(4″-fluorophenyl)-6-hydroxy-1,2,3,4-tetrahydrolsoquinoline;
cis-6-(4-hydroxyphenyl)₅₋₄-(2-piperidin-1-yl-ethoxy)phenyl)-5,6,7,8-tetrahydro-naphthalene-2-ol; and
1-(4′-pyrrolidinolethoxyphenyl)-2-phenyl-6-hydroxy-1,2,3,4-tetrahydroisoquinoline.

Other estrogen agonist/antagonists are described in U.S. Pat. No. 4,133,814 (the disclosure of which is incorporated herein by reference). U.S. Pat. No. 4,133,814 discloses derivatives of 2-phenyl-3-aroyl-benzothiophene and 2-phenyl-3-aroylbenzothiophene-1-oxide.

Other anti-osteoporosis agents, which can be used as the second agent in combination with a compound of the present invention, include, for example, the following: parathyroid hormone (PTH) (a bone anabolic agent); parathyroid hormone (PTH) secretagogues (see, e.g., U.S. Pat. No. 6,132,774), particularly calcium receptor antagonists; calcitonin; and vitamin D and vitamin D analogs.

Any selective androgen receptor modulator (SARM) can be used in combination with a compound of the present invention. A selective androgen receptor modulator (SARM) is a compound that possesses androgenic activity and which exerts tissue-selective effects. SARM compounds can function as androgen receptor agonists, partial agonists, partial antagonists or antagonists. Examples of suitable SARMs include compounds such as cyproterone acetate, chlormadinone, flutamide, hydroxyflutamide, bicalutamide, nilutamide, spironolactone, 4-(trifluoromethyl)-2(1H)-pyrrolidino[3,2-g]quinoline derivatives, 1,2-dihydropyridino[5,6-g]quinoline derivatives and piperidino[3,2-g]quinolinone derivatives.

Cypterone, also known as (1b,2b)-6-chloro-1,2-dihydro-17-hydroxy-3′H-cyclopropa[1,2]pregna-1,4,6-triene-3,20-dione is disclosed in U.S. Pat. No. 3,234,093. Chlormadinone, also known as 17-(acetyloxy)-6-chloropregna-4,6-diene-3,20-dione, in its acetate form, acts as an anti-androgen and is disclosed in U.S. Pat. No. 3,485,852. Nilutamide, also known as 5,5-dimethyl-3-[4-nito-3-(trifluoromethyl)phenyl]-2,4-imidazolidinedione and by the trade name Nilandron® is disclosed in U.S. Pat. No. 4,097,578. Flutamide, also known as 2-methyl-N-[4-nitro-3-(trifluoromethyl)phenyl]propanamide and the trade name Eulexin® is disclosed in U.S. Pat. No. 3,847,988. Bicalutamide, also known as 4′-cyano-a′,a′,a′-trifluoro-3-(4-fluorophenyasulfonyl)-2-hydroxy-2-methylpropiono-m-toluidide and the trade name Casodex® is disclosed in EP-100172. The enantiomers of bicalutamide are discussed by Tucker and Chesterton, J. Med. Chem. 1988, 31, 885-887. Hydroxyflutamide, a known androgen receptor antagonist in most tissues, has been suggested to function as a SARM for effects on IL-6 production by osteoblasts as disclosed in Hofbauer et al. J. Bone Miner. Res. 1999, 14, 1330-1337. Additional SARMs have been disclosed in U.S. Pat. No. 6,017,924; WO 01/16108, WO 01/16133, WO 01/16139, WO 02/00617, WO 02/16310, U.S. Patent Application Publication No. US 2002/0099096, U.S. Patent Application Publication No. US 2003/0022868, WO 03/011302 and WO 03/011824. All of the above references are hereby incorporated by reference herein.

The starting materials and reagents for the above described compounds, are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Some of the compounds of this invention or intermediates in their synthesis have asymmetric carbon atoms and therefore are enantiomers or diastereomers. Diasteromeric mixtures can be separated into their individual diastereomers on the basis of their physical chemical differences by methods known per se, for example, by chromatography and/or fractional crystallization. Enantiomers can be separated by, for example, chiral HPLC methods or converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. Also, an enantiomeric mixture of the compounds or an intermediate in their synthesis which contain an acidic or basic moiety may be separated into their corresponding pure enantiomers by forming a diastereomic salt with an optically pure chiral base or acid (e.g., 1-phenyl-ethyl amine, dibenzyl tartrate or tartaric acid) and separating the diasteromers by fractional crystallization followed by neutralization to break the salt, thus providing the corresponding pure enantiomers. All such isomers, including diastereomers, enantiomers and mixtures thereof are considered as part of this invention for all of the compounds of the present invention, including the compounds of the present invention. Also, some of the compounds of this invention are atropisomers (e.g., substituted biaryls) and are considered as part of this invention.

More specifically, the compounds of this invention may be obtained in enantiomerically enriched form by resolving the racemate of the final compound or an intermediate in its synthesis, employing chromatography (preferably high pressure liquid chromatography [HPLC]) on an asymmetric resin (preferably Chiralcel™ AD or OD (obtained from Chiral Technologies, Exton, Pa.)) with a mobile phase consisting of a hydrocarbon (preferably heptane or hexane) containing between 0 and 50% isopropanol (preferably between 2 and 20%) and between 0 and 5% of an alkyl amine (preferably 0.1% of diethylamine). Concentration of the product containing fractions affords the desired materials.

Some of the compounds of this invention are acidic and they form a salt with a pharmaceutically acceptable cation. Some of the compounds of this invention are basic and they form a salt with a pharmaceutically acceptable anion. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds can be obtained in crystalline form by dissolution in an appropriate solvent(s) such as ethanol, hexanes or water/ethanol mixtures.

In addition, when the compounds of this invention form hydrates or solvates they are also within the scope of the invention.

The compounds of this invention, their prodrugs and the salts of such compounds and prodrugs are all adapted to therapeutic use as agents that inhibit cholesterol ester transfer protein activity in mammals, particularly humans. Thus, the compounds of this invention elevate plasma HDL cholesterol, its associated components, and the functions performed by them in mammals, particularly humans. By virtue of their activity, these agents also reduce plasma levels of triglycerides, VLDL cholesterol, Apo-B, LDL cholesterol and their associated components in mammals, particularly humans. Moreover, these compounds are useful in equalizing LDL cholesterol and HDL cholesterol. Hence, these compounds are useful for the treatment and correction of the various dyslipidemias observed to be associated with the development and incidence of atherosclerosis and cardiovascular disease, including coronary artery disease, coronary heart disease, coronary vascular disease, peripheral vascular disease, hypoalphalipoproteinemia hyperbetalipoproteinemia, hypertriglyceridemia, hypercholesterolemia, familial-hypercholesterolemia, low HDL and associated components, elevated LDL and associated components, elevated Lp(a), elevated small-dense LDL, elevated VLDL and associated components and post-prandial lipemia.

Further, introduction of a functional CETP gene into an animal lacking CETP (mouse) results in reduced HDL levels (Agellon, L. B., et al: J. Biol. Chem. (1991) 266: 10796-10801.) and increased susceptibility to atherosclerosis. (Marotti, K. R., et al: Nature (1993) 364: 73-75.). Also, inhibition of CETP activity with an inhibitory antibody raises HDL-cholesterol in hamster (Evans, G. F., et al: J. of Lipid Research (1994) 35: 1634-1645.) and rabbit (Whitlock, M. E., et al: J. Clin. Invest. (1989) 84: 129-137). Suppression of increased plasma CETP by intravenous injection with antisense oligodeoxynucleotides against CETP mRNA reduced atherosclerosis in cholesterol-fed rabbits (Sugano, M., et al: J. of Biol. Chem. (1998) 273: 5033-5036.) Importantly, human subjects deficient in plasma CETP, due to a genetic mutation possess markedly elevated plasma HDL-cholesterol levels and apolipoprotein A-l, the major apoprotein component of HDL. In addition, most demonstrate markedly decreased plasma LDL cholesterol and apolipoprotein B (the major apolipoprotein component of LDL. (Inazu, A., Brown, M. L., Hesler, C. B., et al.: N. Engl. J. Med. (1990) 323: 1234-1238.)

Given the negative correlation between the levels of HDL cholesterol and HDL associated lipoproteins, and the positive correlation between triglycerides, LDL cholesterol, and their associated apolipoproteins in blood with the development of cardiovascular, cerebral vascular and peripheral vascular diseases, the compounds of this invention, their prodrugs and the salts of such compounds and prodrugs, by virtue of their pharmacologic action, are useful for the prevention, arrestment and/or regression of atherosclerosis and its associated disease states. These include cardiovascular disorders (e.g., angina, ischemia, cardiac ischemia and myocardial infarction), complications due to cardiovascular disease therapies (e.g., reperfusion injury and angioplastic restenosis), hypertension, elevated cardiovascular risk associated with hypertension, stroke, atherosclerosis associated with organ transplantation, cerebrovascular disease, cognitive dysfunction (including, but not limited to, dementia secondary to atherosclerosis, transient cerebral ischemic attacks, neurodegeneration, neuronal deficient, and delayed onset or procession of Alzheimer's disease), elevated levels of oxidative stress, elevated levels of C-Reactive Protein, Metabolic Syndrome and elevated levels of HbA1C.

Because of the beneficial effects widely associated with elevated HDL levels, an agent which inhibits CETP activity in humans, by virtue of its HDL increasing ability, also provides valuable avenues for therapy in a number of other disease areas as well.

Thus, given the ability of the compounds of this invention, their prodrugs and the salts of such compounds and prodrugs to alter lipoprotein composition via inhibition of cholesterol ester transfer, they are of use in the treatment of vascular complications associated with diabetes, lipoprotein abnormalities associated with diabetes and sexual dysfunction associated with diabetes and vascular disease. Hyperlipidemia is present in most subjects with diabetes mellitus (Howard, B. V. 1987. J. Lipid Res. 28, 613). Even in the presence of normal lipid levels, diabetic subjects experience a greater risk of cardiovascular disease (Kannel, W. B. and McGee, D. L. 1979. Diabetes Care 2, 120). CETP-mediated cholesteryl ester transfer is known to be abnormally increased in both insulin-dependent (Bagdade, J. D., Subbalah, P. V. and Ritter, M. C. 1991. Eur. J. Clin. Invest. 21, 161) and non-insulin dependent diabetes (Bagdade. J. D., Ritter, M. C., Lane, J. and Subbalah. 1993. Atherosclerosis 104, 69). It has been suggested that the abnormal increase in cholesterol transfer results in changes in lipoprotein composition, particularly for VLDL and LDL, that are more atherogenic (Bagdade, J. D., Wagner, J. D., Rudel, L. L., and Clarkson, T. B. 1995. J. Lipid Res. 36, 759). These changes would not necessarily be observed during routine lipid screening. Thus the present invention will be useful in reducing the risk of vascular complications as a result of the diabetic condition.

The described agents are useful in the treatment of obesity and elevated cardiovascular risk associated with obesity. In both humans (Radeau, T., Lau, P., Robb, M., McDonnell, M., Ailhaud, G. and McPherson, R., 1995. Journal of Lipid Research. 36 (12):2552-61) and nonhuman primates (Quinet, E., Tall, A., Ramakrishnan, R. and Rudel, L., 1991. Journal of Clinical Investigation. 87 (5):1559-66) mRNA for CETP is expressed at high levels in adipose tissue. The adipose message increases with fat feeding (Martin, L. J., Connelly, P. W., Nancoo, D., Wood, N., Zhang, Z. J., Maguire, G., Quinet, E., Tall, A. R., Marcel, Y. L. and McPherson, R., 1993. Journal of Lipid Research. 34 (3):437-46), and is translated into functional transfer protein and through secretion contributes significantly to plasma CETP levels. In human adipocytes the bulk of cholesterol is provided by plasma LDL and HDL (Fong, B. S., and Angel, A., 1989. Biochimica et Biophysica Acta. 1004 (1):5S60). The uptake of HDL cholesteryl ester is dependent in large part on CETP (Benoist, F., Lau, P., McDonnell, M., Doelle, H., Milne, R. and McPherson, R., 1997. Journal of Biological Chemistry. 272 (38):23572-7). This ability of CETP to stimulate HDL cholesteryl uptake, coupled with the enhanced binding of HDL to adipocytes in obese subjects (Jimenez, J. G., Fong, B., Julien, P., Despres, J. P., Rotstein, L., and Angel, A., 1989. International Journal of Obesity. 13 (5):699-709), suggests a role for CETP, not only in generating the low HDL phenotype for these subjects, but in the development of obesity itself by promoting cholesterol accumulation. Inhibitors of CETP activity that block this process therefore serve as useful adjuvants to dietary therapy in causing weight reduction.

CETP inhibitors are useful in the treatment of inflammation due to Gram-negative sepsis and septic shock. For example, the systemic toxicity of Gram-negative sepsis is in large part due to endotoxin, a lipopolysaccharide (LPS) released from the outer surface of the bacteria, which causes an extensive inflammatory response. Lipopolysaccharide can form complexes with lipoproteins (Ulevitch, R. J., Johnston, A. R., and Weinstein, D. B., 1981. J. Clin. Invest. 67, 827-37). In vitro studies have demonstrated that binding of LPS to HDL substantially reduces the production and release of mediators of inflammation (Ulevitch, R. J., Johhston, A. R., 1978. J. Clin. Invest. 62, 1313-24). In vivo studies show that transgenic mice expressing human apo-Al and elevated HDL levels are protected from septic shock (Levine, D. M., Parker, T. S., Donnelly, T. M., Walsh, A. M., and Rubin, A. L. 1993. Proc. Natl. Acad. Sci. 90, 12040-44). Importantly, administration of reconstituted HDL to humans challenged with endotoxin resulted in a decreased inflammatory response (Pajkrt, D., Doran, J. E., Koster, F., Lerch, P. G., Amet, B., van der Poll, T., ten Cate, J. W., and van Deventer, S. J. H. 1996. J. Exp. Med. 184, 1601-08). The CETP inhibitors, by virtue of the fact that they raise HDL levels, attenuate the development of inflammation and septic shock. These compounds would also be useful in the treatment of endotoxemia, autoimmune diseases and other systemic disease indications, organ or tissue transplant rejection and cancer.

The utility of the compounds of the invention, their prodrugs and the salts of such compounds and prodrugs as medical agents in the treatment of the above described disease/conditions in mammals (e.g. humans, male or female) is demonstrated by the activity of the compounds of this invention in conventional assays and the in vivo assay described below. The in vivo assay (with appropriate modifications within the skill in the art) may be used to determine the activity of other lipid or triglyceride controlling agents as well as the compounds of this invention. Such assays also provide a means whereby the activities of the compounds of this invention, their prodrugs and the salts of such compounds and prodrugs (or the other agents described herein) can be compared to each other and with the activities of other known compounds. The results of these comparisons are useful for determining dosage levels in mammals, including humans, for the treatment of such diseases.

The following protocols can of course be varied by those skilled in the art.

The hyperalphacholesterolemic activity of the compounds can be determined by assessing the effect of these compounds on the action of cholesteryl ester transfer protein by measuring the relative transfer ratio of radiolabeled lipids between lipoprotein fractions, essentially as previously described by Morton in J. Biol. Chem. 25.611992, 1981 and by Dias in Clin. Chem. 34, 2322, 1988.

### CETP In Vitro Assay

The following is a brief description of assays of cholesteryl ester transfer in 97% (whole) or diluted human plasma (in vitro) and animal plasma (ex vivo): CETP activity in the presence or absence of drug is assayed by determining the transfer of ³H-labeled cholesteryl oleate (CO) from exogenous tracer HDL or LDL to the nonHDL or HDL lipoprotein fraction in human plasma, respectively, or from ³H-labeled LDL to the HDL fraction in animal plasma. Labeled human lipoprotein substrates are prepared similarly to the method described by Morton in which the endogenous CETP activity in plasma is employed to transfer ³H—CO from phospholipid liposomes to all the lipoprotein fractions in plasma. ³H-labeled LDL and HDL are subsequently isolated by sequential ultracentrifugation at the density cuts of 1.019-1.063 and 1.10-1.21 g/ml, respectively.

For the 97% or whole plasma activity assay, ³H-labeled HDL is added to plasma at 10-25 nmoles CO/ml and the samples incubated at 37° C. for 2.5-3 hrs. Non-HDL lipoproteins are then precipitated by the addition of an equal volume of 20% (wt/vol) polyethylene glycol 8000 (Dias). The samples are centrifuged 750 g×20 minutes and the radioactivity contained in the HDL-containing supernatant determined by liquid scintillation counting. Introducing varying quantities of the compounds of this invention as a solution in dimethylsulfoxide into human plasma, before addition of the radiolabeled cholesteryl oleate, and comparing the amounts of radiolabel transferred compared to incubations containing no inhibitor compounds allows the cholesteryl ester transfer inhibitory activities to be determined.

When a more sensitive assay is desirable, an in vitro assay using diluted human plasma is utilized. For this assay, ³H-labeled LDL is added to plasma at 50 nmoles CO/ml and the samples incubated at 37° C. for 7 hrs. Non-HDL lipoproteins are then precipitated by the addition of potassium phosphate to 100 mM final concentration followed by manganese chloride to 20 mM final concentration. After vortexing, the samples are centrifuged 750 g×20 minutes and the radioactivity contained in the HDL-containing supernatant determined by liquid scintillation counting. Introducing varying quantities of the compounds of this invention as a solution in dimethylsulfoxide into diluted human plasma, before addition of the radiolabeled cholesteryl oleate, and comparing the amounts of radiolabel transferred compared to incubations containing no inhibitor compounds allows the cholesteryl ester transfer inhibitory activities to be determined. This assay has been adapted to run in microtiter plate format with liquid scintillation counting accomplished using a Wallac plate reader.

Alternatively, the CETP inhibitory activity of compounds can be determined using microtiter plate-based fluorescent transfer assays where the CETP-dependent transfer of a self-quenching cholesteryl ester analog (Bodipy-CE) from human ApoAl-containing emulsion particles to the endogenous lipoproteins in plasma is monitored.

Fluorescent Bodipy-CE donors are prepared by drying down 14 mg of PC, 1.6 mg triolein and 3.5 mg of BODIPY-CE at 60° C. in a vacuum oven and then hydrating the lipids at 80° C. in 12 ml of PBS by probe sonication (at 25% of full power setting) for 2 min under a stream of N₂. The lipid mixture is then cooled to 45° C. and 5 mg (0.125 ?M) of human apolipoprotein Al (from Biodesign, Saco Me.) is added, and again sonicated (at 25% of full power) for 20 min at 45° C., pausing after each minute to allow the probe to cool. The resulting emulsion is spun for 30 min at 3000×g to remove metal probe fragments and then adjusted to 1.12 gm/ml with sodium bromide and layered below a solution of NaBr 1.10 g/ml (16 ml) and subjected to density gradient ultracentrifugation for 24 hours at 50,000-x g to remove unincorporated apolipoprotein Al and small dense particles that remain at the bottom of the gradient. The more buoyant emulsion particles are collected from the top of the gradient and dialyzed in 6 liters (2 changes) of PBS/0.02% azide, and diluted to the appropriate concentrations prior to use.

The CETP-dependent transfer of fluorescent CE analog is monitored in incubations containing the fluorescent human-apolipoprotein Al-containing donor particles, and a source of CETP and acceptor lipoproteins which in these cases are present in diluted human plasma. Bodipy CE fluorescence in the donor particles in the unincubated donor particles is quenched, and the CETP-dependent transfer of Bodipy CE to acceptor particles results in an increase in fluorescence.

When a high sensitivity assay is desired, compounds in 100% dimethyl sulfoxide are tested in a 2.5% plasma 384-well microtiter plate assay. One microliter of compound in 100% dimethyl sulfoxide is added to wells containing 20 ul of 3.75% human plasma (diluted with PBS) using a clonemaster solution transfer device. Transfer is initiated via the addition of 10 ul of 7.5% donors (also diluted with PBS). Following mixing, each plate is taped or placed in a Matripress plate stacker to avoid evaporation and incubated overnight at room temp. (16-20 hrs). Fluorescence is determined on a fluorescent plate reader, 485/530 nm filters, 505 nm dichroic filter. Note that depending upon liquid handling capabilities the intermediate dilutions of plasma and fluorescent donors and the aliquot size of those dilutions can be adjusted as necessary.

When a lower sensitivity assay is desired compounds are tested in a 20% plasma assay that is conceptually similar to the 2.5% assay. Two microliters of compound are added to dry, 96-well, half-area microtiter plates followed by 48 ul of 40% human plasma (diluted in PBS) and 50 ul of 40% donor solution. The fluorescent intensity is monitored after 3 hr incubation at room temperature. In the case of either the 2.5% or the 20% assay, the percent inhibition of CE transfer by compound is calculated by comparing to wells containing fluorescent donors and plasma but no compound.

### CETP In Vivo Assay

Activity of these compounds in vivo can be determined by the amount of agent required to be administered, relative to control, to inhibit cholesteryl ester transfer activity by 50% at various time points ex vivo or to elevate HDL cholesterol by a given percentage in a CETP-containing animal species. Transgenic mice expressing both human CETP and human apolipoprotein Al (Charles River, Boston, Mass.) may be used to assess compounds in vivo. The compounds to be examined are administered by oral gavage in an emulsion vehicle containing 20% (v:v) olive oil and 80% sodium taurocholate (0.5%). Blood is taken from mice retroorbitally before dosing, if a predose blood sample is desirable. At various times after dosing, ranging from 4 h to 24 h, the animals are sacrificed, blood obtained by heart puncture, and lipid parameters measured, including total cholesterol, HDL and LDL cholesterol, and triglycerides. CETP activity is determined by a method similar to that described above except that ³H-cholesteryl oleate-containing LDL is used as the donor source as opposed to HDL. The values obtained for lipids and transfer activity are compared to those obtained prior to dosing and/or to those from mice receiving vehicle alone.

### Plasma Lipids Assay

The activity of these compounds may also be demonstrated by determining the amount of agent required to alter plasma lipid levels, for example HDL cholesterol levels, LDL cholesterol levels, VLDL cholesterol levels or triglycerides, in the plasma of certain mammals, for example marmosets that possess CETP activity and a plasma lipoprotein profile similar to that of humans (Crook et al. Arteriosclerosis 10, 625, 1990). Adult marmosets are assigned to treatment groups so that each group has a similar mean±SD for total, HDL, and/or LDL plasma cholesterol concentrations. After group assignment, marmosets are dosed daily with compound as a dietary admix or by intragastric intubation for from one to eight days. Control marmosets receive only the dosing vehicle. Plasma total, LDL VLDL and HDL cholesterol values can be determined at any point during the study by obtaining blood from an antecubital vein and separating plasma lipoproteins into their individual subclasses by density gradient centrifugation, and by measuring cholesterol concentration as previously described (Crook et al. Arteriosclerosis 10, 625, 1990).

### In Vivo Atherosclerosis Assay

Anti-atherosclerotic effects of the compounds can be determined by the amount of compound required to reduce the lipid deposition in rabbit aorta. Male New Zealand White rabbits are fed a diet containing 0.2% cholesterol and 10% coconut oil for 4 days (meal-fed once per day). Rabbits are bled from the marginal ear vein and total plasma cholesterol values are determined from these samples. The rabbits are then assigned to treatment groups so that each group has a similar mean±SD for total plasma cholesterol concentration, HDL cholesterol concentration, triglyceride concentration and/or cholesteryl ester transfer protein activity. After group assignment, rabbits are dosed daily with compound given as a dietary admix or on a small piece of gelatin based confection. Control rabbits receive only the dosing vehicle, be it the food or the gelatin confection. The cholesterol/coconut oil diet is continued along with the compound administration throughout the study. Plasma cholesterol values and cholesteryl ester transfer protein activity can be determined at any point during the study by obtaining blood from the marginal ear vein. After 3-5 months, the rabbits are sacrificed and the aortae are removed from the thoracic arch to the branch of the iliac arteries. The aortae are cleaned of adventitia, opened longitudinally and then analyzed unstained or stained with Sudan IV as described by Holman et. al. (Lab. Invest. 1958, 7, 42-47). The percent of the lesioned surface area is quantitated by densitometry using an Optimas Image Analyzing System (Image Processing Systems). Reduced lipid deposition is indicated by a reduction in the percent of lesioned surface area in the compound-receiving group in comparison with the control rabbits.

### Antiobesity Protocol

The ability of CETP inhibitors to cause weight loss can be assessed in obese human subjects with body mass index (BMI)≧30 kg/m². Doses of inhibitor are administered sufficient to result in an increase of ≧25% in HDL cholesterol levels. BMI and body fat distribution, defined as waist (V″) to hip (H) ratio (WHR), are monitored during the course of the 36 month studies, and the results for treatment groups compared to those receiving placebo.

### In Vivo Sepsis Assay

In vivo studies show that transgenic mice expressing human apo-Al and elevated HDL levels are protected from septic shock. Thus the ability of CETP inhibitors to protect from septic shock can be demonstrated in transgenic mice expressing both human apo-Al and human CETP transgenes (Levine, D. M., Parker, T. S., Donnelly, T. M., Walsh, A. M. and Rubin, A. L., 1993. Proc. Natl. Acad. Sci. 90, 12040-44). LPS derived from E. coli is administered at 30 mg/kg by i.p. injection to animals which have been administered a CETP inhibitor at an appropriate dose to result in elevation of HDL. The number of surviving mice is determined at times up to 48 h after LPS injection and compared to those mice administered vehicle (minus CETP inhibitor) only.

### In Vivo Blood Pressure Assay

### In Vivo Rabbit Model

Methods: New Zealand White male rabbits (34 kg) are anesthetized with sodium pentobarbital (30 mg/kg, i.v.) and a surgical plane of anesthesia is maintained by a continuous infusion of sodium pentobarbital (16 mg/kg/hr) via an ear vein catheter. A tracheotomy is performed through a ventral midline cervical incision and the rabbits are ventilated with 100% oxygen using a positive pressure ventilator. Body temperature is maintained at 38.5° C. using a heating pad connected to a YSI temperature controller model 72 (Yellow Springs Instruments, Yellow Springs, Md.). Fluid-filled catheters are placed in the right jugular vein (for intravenous drug administration) and in the right carotid artery for arterial pressure monitoring and for blood gas analysis using a model 248 blood gas analyzer (Bayer Diagnostics, Norwood, Mass.). The ventilator is adjusted as needed to maintain blood pH and pCO₂ within normal physiological ranges for rabbits. Arterial pressure is measured using a strain gauge transducer (Spectromed, Oxnard, Calif.), previously calibrated using a mercury manometer, positioned at the level of the heart and connected to the arterial catheter. Arterial pressure signals are digitized at 500 Hz and analyzed using a Po-Ne-Mah Data Acquisition System (Gould Instrument Systems, Valley View, Ohio) to obtain mean arterial pressure and heart rate values. Baseline values are collected when mean arterial pressure and heart rate have stabilized. The test compound is then administered either as a subcutaneous (SC) bolus or as an intravenous (IV) infusion. For subcutaneous (SC) dosing the test compound can be dissolved in an appropriate vehicle such as 5% ethanol in water (5% EtOH: 95% H₂O), while for intravenous dosing the test compound can be dissolved in an appropriate vehicle such as 0.9% normal saline. Arterial pressure and heart rate are monitored continuously for 4 hours following dosing of the test compound or for the duration of a continuous 4 hour infusion of the test compound. Blood is sampled after dosing or during the infusion of the test compound to determine plasma concentrations of the test compounds.

### In Vivo Primate Model

Methods: Adult M. fascicularis primates (6-kg) that have been previously instrumented with subcutaneous vascular access ports in the descending thoracic aorta and conditioned to sit quietly in specially designed primate-restraining chairs are used. All primates are fasted for 12-18 hours prior to the experiment. On the day of the experiment, with the primates restrained in the chairs, a strain gauge pressure transducer (Spectromed, Oxnard, Calif.), previously calibrated using a mercury manometer, is positioned at the level of the heart and connected to the vascular access port to measure arterial pressure. The primates are allowed to acclimate to the chair for at least one hour. Arterial pressure signals are digitized at 500 Hz and continuously recorded throughout the experiment and analyzed using a Po-Ne-Mah Data Acquisition System (Gould Instrument Systems, Valley View, Ohio) to obtain the measurements of mean arterial pressure and heart rate. Baseline values are collected when the primates are sitting calmly and when mean arterial pressure and heart rate have stabilized. The test compound is then administered as a subcutaneous (SC) bolus of a solution of the test compound in an appropriate vehicle such as 5% ethanol in water (5% EtOH: 95% H₂O). The solution of test compound or vehicle is filtered through a 0.22 micron filter prior to injection and a typical dosing volume is 0.2 ml/kg. Arterial pressure and heart rate are monitored continuously for 4 hours following dosing of the test compound and are recorded at selected time intervals for data comparison (vehicle vs test compound). Blood samples (1.5 ml) are withdrawn to determine plasma concentrations of the test compound and withdrawn blood is immediately replaced with 0.9% sterile saline to maintain blood volume.

Administration of the compounds of this invention can be via any method which delivers a compound of this invention systemically and/or locally. These methods include oral routes, parenteral, intraduodenal routes, etc. Generally, the compounds of this invention are administered orally, but parenteral administration (e.g., intravenous, intramuscular, subcutaneous or intramedullary) may be utilized, for example, where oral administration is inappropriate for the target or where the patient is unable to ingest the drug.

In general an amount of a compound of this invention is used that is sufficient to achieve the therapeutic effect desired (e.g., HDL elevation).

In general an effective dosage for the compounds of this invention is about 0.001 to 100 mg/kg/day of the compound, a prodrug thereof, or a pharmaceutically acceptable salt of said compound or of said prodrug. An especially preferred dosage is about 0.01 to 10 mg/kg/day of the compound, a prodrug thereof, or a pharmaceutically acceptable salt of said compound or of said prodrug.

A dosage of the combination pharmaceutical agents to be used in conjunction with the CETP inhibitors is used that is effective for the indication being treated.

For example, typically an effective dosage for HMG-CoA reductase inhibitors is in the range of 0.01 to 100 mg/kg/day. In general an effect dosage for a PPAR modulator is in the range of 0.01 to 100 mg/kg/day.

The compounds of the present invention are generally administered in the form of a pharmaceutical composition comprising at least one of the compounds of this invention together with a pharmaceutically acceptable vehicle, diluent or carrier as described below. Thus, the compounds of this invention can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. A preferred formulation is a solution or suspension in an oil, for example, a vegetable oil, such as olive oil; triglycerides such as those marketed under the name, Miglyol™; or mono- or diglycerides such as those marketed under the name, Capmul™, for example, in a soft gelatin capsule. Antioxidants may be added to prevent long-term degradation as appropriate. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

Pharmaceutical compositions comprising a solid amorphous dispersion of a cholesteryl ester transfer protein (CETP) inhibitor and a concentration-enhancing polymer are described in International Publication No. WO 02/11710, which is hereby incorporated by reference herein. Self-emulsifying formulations of cholesteryl ester transfer protein (CETP) inhibitors are described in International Publication No. WO 03/000295, which is hereby incorporated by reference herein. Methods for depositing small drug crystals on excipients are set forth in the literature, such as in J. Pharm. Pharmacol. 1987, 39:769-773, which is hereby incorporated by reference herein.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the disease/condition of the subject being treated, e.g., atherosclerosis.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of the present invention, a prodrug thereof or a salt of such compound or prodrug and a second compound as described above. The kit comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of compounds of the present invention can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of this invention either alone or in combination with each other or other compounds generally will be administered in a convenient formulation. The following formulation examples only are illustrative and are not intended to limit the scope of the present invention.

In the formulations which follow, “active ingredient” means a compound of this invention.

### Formulation 1: Gelatin Capsules

Hard gelatin capsules are prepared using the following:

| Ingredient | Quantity (mg/capsule) |
|---|---|
| Active ingredient | 0.25-100 |
| Starch, NF | 0-650 |
| Starch flowable powder | 0-50 |
| Silicone fluid 350 centistokes | 0-15 |

A tablet formulation is prepared using the ingredients below:

### Formulation 2: Tablets

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-100 mg of active ingredients are made up as follows:

### Formulation 3: Tablets

| Ingredient | Quantity (mg/tablet) |
|---|---|
| Active ingredient | 0.25-100 |
| Starch | 45 |
| Cellulose, microcrystalline | 35 |
| Polyvinylpyrrolidone (as 10% solution in water) | 4 |
| Sodium carboxymethyl cellulose | 4.5 |
| Magnesium stearate | 0.5 |
| Talc | 1 |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 ml dose are made as follows:

### Formulation 4: Suspensions

| Ingredient | Quantity (mg/5 ml) | |
|---|---|---|
| Active ingredient | 0.25-100 | mg |
| Sodium carboxymethyl cellulose | 50 | mg |
| Syrup | 1.25 | mg |
| Benzoic acid solution | 0.10 | mL |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form smooth paste. The benzoic acid solution, flavor, and color are diluted with some of the water and added, with stirring. Sufficient water is then added to produce the required volume.

An aerosol solution is prepared containing the following ingredients:

### Formulation 5: Aerosol

| Ingredient | Quantity (% by weight) |
|---|---|
| Active ingredient | 0.25 |
| Ethanol | 25.75 |
| Propellant 22 (Chlorodifluoromethane) | 70.00 |

The active ingredient is mixed with ethanol and the mixture added to a portion of the propellant 22, cooled to 30° C., and transferred to a filling device. The required amount is then fed to a stainless steel container and diluted with the remaining propellant. The valve units are then fitted to the container.

Suppositories are prepared as follows:

### Formulation 6: Suppositories

| Ingredient | Quantity (mg/suppository) |
|---|---|
| Active ingredient | 250 |
| Saturated fatty acid glycerides | 2,000 |

The active ingredient is passed through a No. 60 mesh U.S. sieve and suspended in the saturated fatty acid glycerides previously melted using the minimal necessary heat. The mixture is then poured into a suppository mold of nominal 2 g capacity and allowed to cool.

An intravenous formulation is prepared as follows:

### Formulation 7: Intravenous Solution

| Ingredient | Quantity | |
|---|---|---|
| Active ingredient dissolved in ethanol 1% | 20 | mg |
| Intralipid ™ emulsion | 1,000 | mL |

The solution of the above ingredients is intravenously administered to a patient at a rate of about 1 mL per minute.

Soft gelatin capsules are prepared using the following:

Formulation 8: Soft Gelatin Capsule with Oil Formulation

| Ingredient | Quantity (mg/capsule) |
|---|---|
| Active ingredient | 10-500 |
| Olive Oil or Miglyol ™ Oil | 500-1000 |

The active ingredient above may also be a combination of agents.

### General Experimental Procedures

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification. Room or ambient temperature refers to 20-25° C. All non-aqueous reactions were run under a nitrogen atmosphere for convenience and to maximize yields. Concentration in vacuo means that a rotary evaporator was used. The names for the compounds of the invention were created by the Autonom 2.0 PC-batch version from Beilstein Informationssysteme GmbH (ISBN 3-89536-976-4). The chemical structures depicted may be only exemplary of the general structure or of limited isomers, and not include specific stereochemistry as recited in the chemical name.

NMR spectra were recorded on a Varian Unity 400 (Varian Co., Palo Alto, Calif.) NMR spectrometer at ambient temperature. Chemical shifts are expressed in parts per million (6) relative to an external standard (tetramethylsilane). The peak shapes are denoted as follows: s, singlet; d, doublet, t, triplet, q, quartet, m, multiplet with the prefix br indicating a broadened signal. The coupling constant (J) data given have a maximum error of +0.41 Hz due to the digitization of the spectra that are acquired. Mass spectra were obtained by (1) atmospheric pressure chemical ionization (APCI) in alternating positive and negative ion mode using a Fisons Platform II Spectrometer or a Micromass MZD Spectrometer (Micromass, Manchester, UK) or (2) electrospray ionization in alternating positive and negative ion mode using a Micromass MZD Spectrometer (Micromass, Manchester, UK) with a Gilson LC-MS interface (Gilson Instruments, Middleton, Wis.) or (3) a QP-8000 mass spectrometer (Shimadzu Corporation, Kyoto, Japan) operating in positive or negative single ion monitoring mode, utilizing electrospray ionization or atmospheric pressure chemical ionization. Where the intensity of chlorine- or bromine-containing ions are described, the expected intensity ratio was observed (approximately 3:1 for ³⁵Cl/³⁷Cl-containing ions and 1:1 for ⁷⁹Br/⁸¹Br-containing ions) and the position of only the lower mass ion is given.

Column chromatography was performed with either Baker Silica Gel (40 μm) (J. T. Baker, Phillipsburg, N. J.) or Silica Gel 60 (40-63 μm) (EM Sciences, Gibbstown, N. J.). Flash chromatography was performed using a Flash 12 or Flash 40 column (Biotage, Dyar Corp., Charlottesville, Va.). Radial chromatography was performed using a chromatotron Model 7924T (Harrison Research, Palo Alto, Calif.). Preparative HPLC purification was performed on a Shimadzu 10A preparative HPLC system (Shimadzu Corporation, Kyoto, Japan) using a model SIL-10A autosampler and model 8A HPLC pumps.

Preparative HPLC purification was performed on a Waters Fractionlynx LC/MS/UV system (Waters Corporation; Milford, Mass., USA) equipped with model 2767 injector/collector, model 2525 high flow binary pump modified by a model 515 low flow pump, a model 515 low flow pump for makeup flow, model GS splitter, model ZQ single quad mass spectrometer on the low flow side, model 996 photodiode array UV detector on the high flow side in pre-collector configuration, and a model 2487 dual UV detector on the high flow side in post-collector configuration. Fraction trigger is performed by the ZQ detector in electrospray positive (ESI+) ionization mode operating on single mass triggering. Chromatography methods are either 0.05% trifluoroacetic acid or 0.1% ammonia modified acetonitrile-water gradients. In the case of acid modified gradients Waters Symmetry C8 or C18 (19×50 mm; 5 um) are typically used and in basic conditions Waters Xterra MS C8 or MS C18 (19×50 mm; 5 um).

Optical rotations were determined using a Jasco P-1020 Polarimeter Jasco Inc., Easton, Md.)

Dimethylformamide (“DMF”), tetrahydrofuran (“THF”), toluene and dichloromethane (“DCM”) were the anhydrous grade supplied by Aldrich Chemical Company (Milwaukee, Wis.). Unless otherwise specified, reagents were used as obtained from commercial sources. The terms “concentrated” and “evaporated” refer to removal of solvent at 1-200 mm of mercury pressure on a rotary evaporator with a bath temperature of less than 45° C. The abbreviation “min” stand for “minutes” and “h” or “hr” stand for “hours.” The abbreviation “gm” or “g” stand for grams. The abbreviation “μl” or “μL” stand for microliters.

### Preparation 1

### 2-bromo-5-(trifluoromethyl)benzoic acid

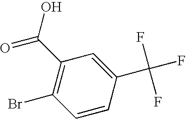

To a solution of n-BuLi (26.7 mL of 2.5 M solution in Tetrahydrofuran (THF), 66.7 mmol) in THF (130 mL) at −78° C. was added 2,2,6,6-Tetramethylpiperidine (22.5 mL, 133.4 mmol). The mixture was stirred at −78° C. for 30 minutes, and then carefully lowered to −100° C. using liquid nitrogen. Neat 1-bromo-4-trifluoromethyl)benzene (15 g, 66.7 mmol) was added. The mixture was kept at −100° C. for 6 hours, and poured onto freshly crushed dry ice. The resulting mixture was stirred at room temperature for 16 hours. The residue solvent was removed by evaporation. Water (150 mL) was added and the mixture was extracted with diethyl ether (three times at 50 mL). The aqueous layer was acidified using concentrated hydrochloric acid (HCl), extracted with methylene chloride (three times at 50 mL). The combined organic layers were washed with saturated sodium chloride (NaCl) (75 ml), dried with magnesium sulfate (MgSO₄), filtered and concentrated to yield the title compound as a white solid (5.41 g). ¹H NMR (300 MHz, CHLOROFORM-D) δ ppm 7.7 (dd, J=8.4, 2.3 Hz, 1H) 7.9 (d, J=8.4 Hz, 1H) 8.3 (d, J=2.0 Hz, 1H); MS (ES+) Calc: 267.93. Found: 266.7 (M−1).

### Preparation 2

### (2-bromo-5-(trifluoromethyl)phenyl)methanol

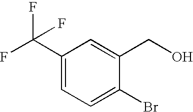

To an ice-cooled solution of 2-bromo-5-(trifluoromethyl)benzoic acid (5.16 g, 19 mmol) in THF (50 mL) was added borane-tetrahydrofuran complex (70 mL of 1M solution in THF, 70 mmol). The resulting mixture was stirred at room temperature for 16 hours. The reaction mixture was quenched with methanol. Solvent was removed. The residue was partitioned between ethyl acetate (three times at 40 mL) and 1M sodium bicarbonate (50 mL). The combined organic layers were washed with saturated NaCl (50 mL), dried (magnesium sulfate) and concentrated to yield the title compound as an oil (4.85 g). ¹H NMR (300 MHz, CHLOROFORM-D) δ ppm 4.8 (s, 2H) 7.5 (m, 1H) 7.7 (d, J=8.2 Hz, 1H) 7.8 (d, J=1.6 Hz, 1H).

### Preparation 3

### 1-bromo-2-(bromomethyl)-4-(trifluoromethyl)benzene

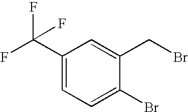

To a solution of (2-bromo-5-(trifluoromethyl)phenyl)methanol (4.7 g, 18 mmol) in methylene chloride (50 mL) at −10° C. was added carbon tetrabromide (CBr₄) (7.17 g, 21.6 mmol). The resulting mixture was stirred at −10° C. for 15 minutes. Triphenylphosphine (5.61 g, 21.4 mmol) was then slowly added portion-wise. This mixture was stirred at room temperature for 16 hours. The mixture was partitioned between saturated. Ammonium chloride (NH₄Cl) (50 ml), and methylene chloride (2×50 mL). The combined organic layers were washed with saturated NaCl (50 mL), dried (magnesium sulfate), and concentrated. The residue was purified by flash chromatography (Silica gel) (eluted with 3:1 hexanes-ethyl acetate) to yield the title compound as a white solid (4.01 g). ¹H NMR (300 MHz, CHLOROFORM-D) δ ppm 4.6 (s, 2H) 7.5 (dd, J=8.3, 1.6 Hz, 1H) 7.8 (m, 2H).

### Preparation 4

### 2-methyl-2H-tetrazol-5-amine

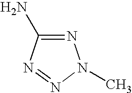

To 2H-tetrazol-5-amine (50 g, 0.59 mol) in sodium hydroxide (NaOH) (118 mL of 5.125 M solution, 0.6 mol) was slowly added dimethyl sulfate (38 g, 0.3 mol), not allowing temperature to go above 95° C. The resulting mixture was stirred at 95° C. for 1 hour. The reaction was cooled to 5° C., and kept at 5° C. for 16 hours. The precipitate was filtered. The resultant filtrate was concentrated, and the residue was recrystallized in 85% toluene/ethanol (100 mL). The solid, which formed was collected, and recrystallized from toluene (13 mL). The subsequent precipitate was collected, and recrystallized from chloroform. The resultant solid was filtered to give 5-methyl-2H-tetrazol-5-amine, and the filtrate was concentrated to yield the title compound (15 g). ¹H NMR (300 MHz, CHLOROFORM-D) δ ppm 6.0 (s, 2H), 4.1 (s, 3H).

### Preparation 5

### N-(3,5-bis(trifluoromethyl)benzyl)-2-methyl-2H-tetrazol-5-amine

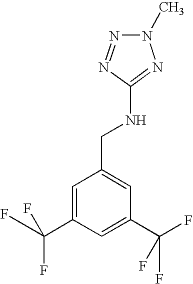

A mixture of 3,5-bis(trifluoromethyl)benzaldehyde (4 g, 16.5 mmol), 2-methyl-2H-tetrazol-5-amine (1.96 g, 19.8 mmol) and Molecular Sieves (5-10 Å beads) in toluene (50 mL) was heated at reflux for 4 hours, after which time the solvent was removed. Ethanol (50 mL) and sodium borohydride (1.25 g, 33 mmol) were added. The resulting mixture was stirred at room temperature for 30 minutes, and then partitioned between saturated ammonium chloride (50 mL) and ethyl acetate (twice at 50 mL). The combine organic layers were washed with saturated NaCl (50 mL), dried (magnesium sulfate), filtered and concentrated to yield the title compound as a white solid (4.7 g). ¹H NMR (300 MHz, CHLOROFORM-D) δ ppm 4.2 (s, 3H) 4.7 (s, 1H) 4.7 (s, 1H) 5.0 (t, J=6.0 Hz, 1H) 7.8 (s, 1H) 7.9 (s, 2H); MS (ES⁺) Calc: 325.08. Found: 325.8 (M+1).

### Preparation 6

### N-(2-bromo-5-(trifluoromethyl)benzyl)-N-(3,5-bis(trifluoromethyl)benzyl)-2-methyl-2H-tetrazol-5-amine

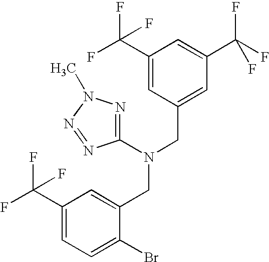

To a solution of N-(3,5-bis(trifluoromethyl)benzyl)-2-methyl-2H-tetrazol-5-amine (3.9 g, 12 mmol) in THF (50 mL) at room temperature was added potassium tert-butoxide (KOtBu) (13.2 ml of 1 M solution, 13.2 mmol) followed by 1-bromo-2-bromomethyl)-4-(trifluoromethyl)benzene (4 g, 12.6 mmol). The mixture was stirred at room temperature for 16 hours. Additional KOtBu in THF (13.2 mL of 1 M solution, 13.2 mmol) was added, and the mixture was stirred at room temperature for 2 hours. The reaction mixture was partitioned between water (50 mL) and ethyl acetate (three times at 50 mL). The combined organic layers were washed with saturated NaCl (50 mL), dried (magnesium sulfate), and concentrated. The residue was purified by flash chromatography (Silica gel) (eluted with 3:1 hexane-ethyl acetate) to yield the title compound (4.72 g). ¹H NMR (300 MHz, CHLOROFORM-D) δ ppm 4.2 (s, 3H) 4.8 (s, 2H) 4.9 (s, 2H) 7.4 (dd, J=8.2, 1.7 Hz, 1H) 7.5 (d, J=1.7 Hz, 1H) 7.7 (m, 3H) 7.8 (s, 1H); MS (ES⁺) Calc: 561.02. Found: 561.7 (M+1).

### Preparation 7

### N-(3,5-bis(trifluoromethyl)benzyl)-N-(5-trifluoromethyl)-2-(4,5,5-tetramethyl-1,3,2-dioxaborolan-2-yl)benzyl)-2-methyl-2H-tetrazol-5-amine

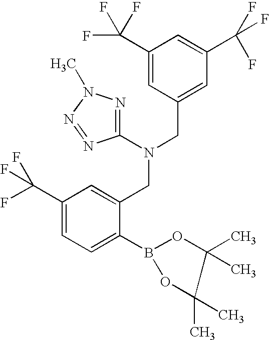

To a flame-dried flask charged with N-(2-bromo-5-(trifluoromethyl)benzyl)-N-3,5-bis(trifluoromethyl)benzyl)-2-methyl-2H-tetrazol-5-amine (561 mg, 1 mmol) was added dimethyl sulfoxide (DMSO) (5 mL) followed by 4,4,5,5-tetramethyl-2-(4,4,5,5-tetramethyl-1,3,2-dioxaborolan-2-yl)-1,3,2-dioxaborolane (304.8 mg, 1.2 mmol) and potassium acetate (KOAc) (294.5 mg, 3 mmol). The resulting mixture was purged with nitrogen (N₂). [1,1′-Bis)diphenylphosphino)ferrocene]dichloropalldium(II), complex with dichloromethane (163.33 mg, 0.2 mmol) was added. The mixture was heated at 80° C. for 3 hours. The reaction mixture was quenched with water (20 mL), and extracted with ethyl acetate (three times at 50 mL). The combined organic layers were washed with saturated NaCl, dried sodium sulfate (Na₂SO₄), and concentrated. The residue was purified by flash chromatography (Silica gel) (eluted with 1-5% ethyl acetate in toluene) to afford the title compound as a paste (270 mg). ¹H NMR (400 MHz, CHLOROFORM-D) δ ppm 1.2 (s, 12H) 4.2 (s, 3H) 4.7 (s, 2H) 5.1 (s, 2H) 7.5 (d, J=7.7 Hz, 1H) 7.5 (s, 1H) 7.6 (s, 2H) 7.7 (s, 1H) 7.9 (d, J=7.7 Hz, 1H); MS (ES+) Calc: 609.12. Found: 610.2 (M+1).

### Preparation 8

### (2-Bromo-5-trifluoromethyl-pyridin-3-yl)-methanol

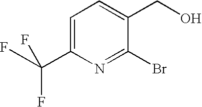

To a solution of methyl 2-bromo-5-(trifluoromethyl)pyridine-3-carboxylate (7.5 g, 26.4 mmol) in THF (100 mL) at −78° C. was slowly added diisobutylaluminum hydride (DIBAL-H) (61 mL of 1M solution, 61 mmol). The mixture was slowly warmed to room temperature over 2 hours. The reaction mixture was partitioned between 10% w/v citric acid (50 mL) and ethyl acetate (twice at 50 mL). The combined organic layers were washed with saturated NaCl (50 mL), dried (magnesium sulfate) and concentrated to yield the title compound as a white solid (7.3 g). ¹H NMR (300 MHz, CHLOROFORM-D) δ ppm 4.8 (s, 2H) 7.7 (d, J=7.8 Hz, 1H) 8.1 (dd, J=7.9, 0.7 Hz, 1H); MS (ES+) Calc: 254.95. Found: 255.7 (M+1).

### Preparation 9

### 2-Bromo-3-bromomethyl-5-trifluoromethyl-pyridine

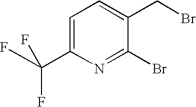

To a solution of (2-Bromo-5-trifluoromethyl-pyridin-3-yl)-methanol (6.53 g, 25.5 mmol) in methylene chloride (100 mL) at −10° C. was added CBr₄ (10.58 g, 31.9 mmol). The mixture was stirred at −10° C. for 15 minutes, and triphenylphosphine (8.02 g, 30.6 mmol) was slowly added. The resulting solution was stirred at room temperature overnight, quenched with Saturated ammonium chloride (50 mL) and extracted with methylene chloride (twice at 100 mL). The combined organic layers were washed with Saturated NaCl (50 mL), dried (magnesium sulfate) and concentrated. The residue was purified by flash chromatography (Silica gel) (eluted with 9:1 hexane-ethyl acetate to yield the title compound as a white solid (3.42 g). ¹H NMR (300 MHz, CHLOROFORM-D) δ ppm 4.6 (s, 2H) 7.7 (d, J=7.8 Hz, 1H) 8.0 (d, J=7.8 Hz, 1H).

### Preparation 10

### (3,5-Bis-trifluoromethyl-benzyl)-(2-bromo-6-trifluoromethyl-pyridin-3-ylmethyl)-(2-methyl-2H-tetrazol-5-yl)amine

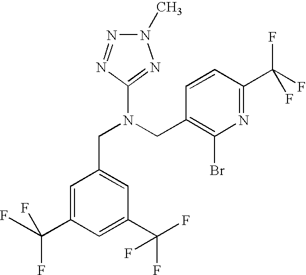

To a solution of N-(3,5-bis(trifluoromethyl)benzyl)-2-methyl-2H-tetrazol-5-amine (1 g, 3.08 mmol) in THF (20 mL) was added KOtBu (3.4 mL of 1 M solution, 3.4 mmol) followed by 2-bromo-3-bromomethyl-5-trifluoromethyl-pyridine (1.08 g, 3.4 mmol). The resulting mixture was stirred at room temperature for 16 hours. The reaction mixture was partitioned between water (50 mL) and ethyl acetate (twice at 40 mL). The combined organic layers were washed with saturated NaCl (50 mL), dried (magnesium sulfate) and concentrated. The residue was purified by flash chromatography (Silica gel) (eluted with 3:1 hexane-ethyl acetate) to yield the title compound as a yellow solid (1.3 g). ¹H NMR (300 MHz, CHLOROFORM-D) δ ppm 4.2 (s, 3H) 4.8 (s, 2H) 4.9 (s, 2H) 7.6 (d, J=7.9 Hz, 1H) 7.7 (d, J=7.9 Hz, 1H) 7.8 (s, 2H) 7.8 (s, 1H); MS (ES+) Calc: 562.02. Found: 562.7 (M+1).

### Preparation 11

### 1-(2-{[(3,5-Bis-trifluoromethyl-benzyl)-(2-methyl-2H-tetrazol-5-yl)-amino]-methyl}-4-trifluoromethyl-phenyl)-propan-1-ol

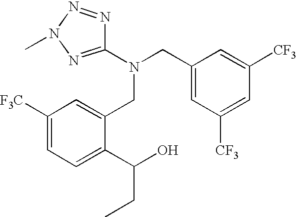

To a solution of N-(2-bromo-5-(trifluoromethyl)benzyl)-N-(3,5-bis(trifluoromethyl)benzyl)-2-methyl-2H-tetrazol-5-amine (70 mg, 0.125 mmol) in THF (0.5 mL) was added isopropylmagnesium chloride solution (0.125 mL of 2 M solution in THF, 0.25 mmol). The mixture was stirred at room temperature for 6 hours, and propionaldehyde (100 μL) was added. The reaction was monitored by TLC. Additional isopropylmagnesium chloride (200 μL) was added. The mixture was stirred at room temperature for 6 hours and propionaldehyde (100 μL) was added. The reaction was quenched with ammonium chloride. Ethyl acetate was added. The organic layer was filtered through magnesium sulfate and evaporated. The residue was purified by flash chromatography on 12 gm Silica gel column to yield the title compound (21 mg). ¹H NMR (300 MHz, CHLOROFORM-D) δ ppm 0.9 (t, J=7.4 Hz, 3H) 2.4 (m, 2H) 4.2 (s, 3H) 4.8 (m, 4H) 5.1 (m, 1H) 7.4 (s, 1H) 7.6 (d, J=9.2 Hz, 1H) 7.7 (m, 3H) 7.8 (s, 1H); MS (ES+) Calc: 541.15. Found: 542.1 (M+1).

### Preparation 12

### 5′-Isopropyl-2′-methoxy-4-trifluoromethyl-biphenyl-2-carbonitrile

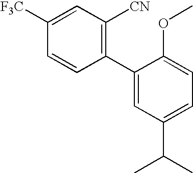

A flame-dried 500 ml flask equipped with a magnetic stirring bar and a condenser was charged with 2-chloro-5-(trifluoromethyl)benzonitrile (15 g, 73 mmol), 5 isopropyl-2-methoxyphenylboronic acid (14.17 g, 73 mmol), potassium fluoride (12.7 g, 219 mmol) and 1,4-dioxan (150 mL). The resulting mixture was purged with Nitrogen (N₂). Tri-tert-butylphosphine tetrafluoroborate adduct (2.12 g, 7.3 mmol) and tris(dibenzyllideneacetone)dipalladium(0) (3.34 g, 3.65 mmol) were added and the mixture was purged with N₂ again. The mixture was then heated and stirred at 110° C. overnight. Solvent was removed in vacuo. The residue was partitioned between 1M sodium hydroxide (200 mL) and diethyl ether (200 mL). The organic layer was collected and washed with saturated NaCl, dried over sodium sulfate (Na₂SO₄), and concentrated under reduced pressure to give crude product as an oil. Purification by chromatography on silica gel (1-5% ethyl acetate in hexane) give the titled compound (23.2 g, 92%) as a clear oil. ¹H NMR (400 MHz, CHLOROFORM-D) δ ppm 1.3 (d, J=7.0 Hz, 6H) 2.9 (m, 1H) 3.8 (s, 3H) 7.0 (d, J=8.5 Hz, 1H) 7.1 (d, J=2.5 Hz, 1H) 7.3 (dd, J=8.5, 1.9 Hz, 1H) 7.6 (d, J=8.3 Hz, 1H) 7.9 (dd, J=8.2, 1.3 Hz, 1H) 8.0 (d, J=2.1 Hz, 1H).

### Preparation 13

### 5′-Isopropyl-2′-methoxy-4-trifluoromethyl-biphenyl-2-carbaldehyde

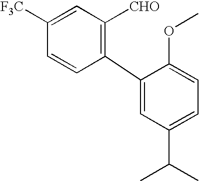

To a stirring solution of 5′-Isopropyl-2′-methoxy-4-trifluoromethyl-biphenyl-2-carbonitrile (10 g, 31.32 mmol) in methylene chloride (200 mL) was added DIBAL-H (78 mL of 1 M solution in toluene) slowly at room temperature. The resulting solution was stirred at room temperature for 30 minutes and then cooled to 0° C. 3N hydrochloric acid (100 mL) was added very carefully to quench the reaction. The mixture was stirred at 0° C. and then room temperature for 2 hours. Diethyl ether (50 mL) was added to the mixture. The organic layer was collected, washed with saturated NaCl, water, dried (sodium sulfate), and concentrated under reduced pressure. The residue was purified by chromatography on silica gel (0-5% ethyl acetate in hexane) to afford the titled compound (6.05 g, 60%) as an oil. ¹H NMR (400 MHz, CHLOROFORM-D) δ ppm 1.3 (d, J=7.0 Hz, 6H) 2.9 (m, 1H) 3.7 (s, 3H) 6.9 (d, J=8.5 Hz, 1H) 7.2 (d, J=2.3 Hz, 1H) 7.3 (dd, J=8.5, 2.3 Hz, 1H) 7.5 (d, J=8.1 Hz, 1H) 7.9 (dd, J=8.1, 1.5 Hz, 1H) 8.3 (d, J=2.1 Hz, 1H) 9.8 (s, 1H)

### Preparation 14

### (3,5-Bis-trifluoromethyl-benzyl)-(5′-isopropyl-2′-methoxy-4-trifluoromethyl-biphenyl-2-ylmethyl)-amine

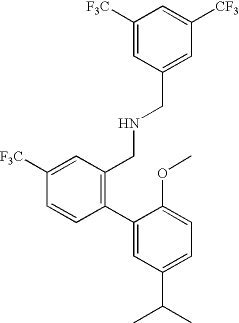

To a solution of 5′-Isopropyl-2′-methoxy-4-trifluoromethyl-biphenyl-2-carbaldehyde (200 mg, 0.62 mmol) in ethanol (50 mL) at room temperature was added 3,5-bis(trifluoromethyl)benzylamine (151 mg, 0.62 mmol). The resulting solution was stirred at room temperature for 2 hours before sodium borohydride (94.24 mg, 2.48 mmol) was added. The resulting mixture was stirred at room temperature for another 2 hours. Solvent was removed in vacuo. The residue was partitioned between saturated sodium bicarbonate sodium bicarbonate solution (100 mL) and methylene chloride (100 mL). The organic layer was collected, dried over sodium sulfate (Na₂SO₄), and concentrated under reduced pressure. The residue was purified by chromatography on silica gel (10% ethyl acetate in hexane) to afford the titled compound (324 mg, 95%) as a white solid. ¹H NMR (400 MHz, CHLOROFORM-D) δ ppm 1.2 (d, J=6.8 Hz, 6H) 2.9 (m, 1H) 3.7 (s, 3H) 3.7 (m, 4H) 6.9 (d, J=8.5 Hz, 1H) 7.0 (d, J=2.3 Hz, 1H) 7.2 (dd, J=8.1, 1.9 Hz, 1H) 7.3 (d, J=7.9 Hz, 1H) 7.6 (dd, J=8.0, 1.3 Hz, 1H) 7.7 (s, 2H) 7.7 (s, 1H) 7.8 (s, 1H).

### Preparation 15

### (3,5-Bis-trifluoromethyl-benzyl)-(2-bromo-benzyl)-(2-methyl-2H-tetrazol-5-yl)-amine

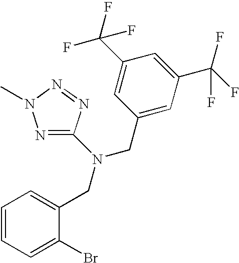

The title compound was prepared using procedures analogous to those described above for the synthesis of N-(2-bromo-5-(trifluoromethyl)benzyl)-N-(3,5-bis(trifluoromethyl)benzyl)-2-methyl-2H-tetrazol-5-amine (Preparation 6) using 1-bromo-2-(bromomethyl)benzene as starting material. ¹H NMR (300 MHz, CHLOROFORM-D) δ ppm 4.2 (s, 3H) 4.8 (s, 2H) 4.8 (s, 2H) 7.1 (m, 1H) 7.2 (s, 1H) 7.2 (s, 1H) 7.5 (d, J=7.8 Hz, 1H) 7.7 (s, 2H) 7.7 (s, 1H); MS (ES+) Calc: 493.03. Found: 493.9 (M+1).

### Preparation 16

### (5′-Isopropyl-2′-methoxy-4-trifluoromethyl-biphenyl-2-yl)-methanol

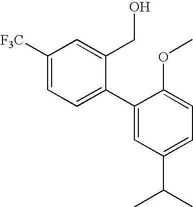

To a solution of 5′-Isopropyl-2′-methoxy trifluoromethyl-biphenyl-2-carbaldehyde (1.09 g, 3.39 mmol) in ethanol (10 mL) at 0° C. was slowly added sodium borohydride (142 mg, 3.73 mmol). The mixture was stirred at room temperature for 2 hours. The reaction was then carefully quenched with 1M hydrochloric acid and extracted with ethyl acetate. The combined organic layers were washed with saturated NaCl, dried (magnesium sulfate), and solvent was removed to yield the title compound as a white solid/gum. ¹H NMR (300 MHz, CHLOROFORM-D) δ ppm 1.3 (d, J=7.0 Hz, 6H) 2.9 (m, 1H) 3.8 (s, 3H) 4.5 (d, J=13.4 Hz, 1H) 4.6 (d, J=12.1 Hz, 1H) 7.0 (d, J=8.6 Hz, 1H) 7.0 (d, J=2.3 Hz, 1H) 7.3 (dd, J=8.4, 2.5 Hz, 1H) 7.4 (d, J=7.9 Hz, 1H) 7.6 (dd, J=8.0, 1.5 Hz, 1H) 7.9 (s, 1H); MS (ES⁺) Calc: 324.13. Found: 369.1 (M+45).

### Preparation 17

### 2-Bromomethyl-5′-isoproplyl-2′-methoxy-4-trifluoromethyl-biphenyl

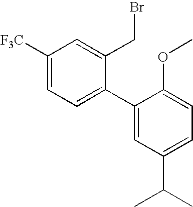

To a solution of (5′-Isopropyl-2′-methoxy-4-trifluoromethyl-biphenyl-2-yl)-methanol (700 mg, 2.16 mmol) and CBr₄ (861 mg, 2.6 mmol) in methylene chloride (10 mL) at −10° C. was added triphenylphosphine (676 mg, 2.57 mmol). The mixture was stirred at room temperature for 16 hours. The reaction mixture was partitioned between saturated ammonium chloride and methylene chloride (twice with 10 mL). The combined organic layers were washed with saturated NaCl (10 mL), dried (magnesium sulfate) and concentrated. The residue was purified by column chromatography (eluted with 3:1 hexane-ethyl acetate) to yield the title compound as a colorless oil (562 mg). ¹H NMR (300 MHz, CHLOROFORM-D) δ ppm 1.3 (d, J=6.8 Hz, 6H) 2.9 (m, 1H) 3.8 (s, 3H) 4.3 (d, J=10.3 Hz, 1H) 4.5 (d, J=10.4 Hz, 1H) 7.0 (d, J=8.6 Hz, 1H) 7.1 (d, J=2.5 Hz, 1H) 7.3 (dd, J=8.4, 3.0 Hz, 1H) 7.4 (d, J=7.9 Hz, 1H) 7.6 (dd, J=8.0, 1.2 Hz, 1H) 7.8 (d, J=0.8 Hz, 1H)

### Example 1

### N-(3,5-bis(trifluoromethyl)benzyl)-N-((5-(trifluoromethyl)-2-(naphthalen-1-yl)phenyl)methyl)-2-methyl-2H-tetrazol-5-amine

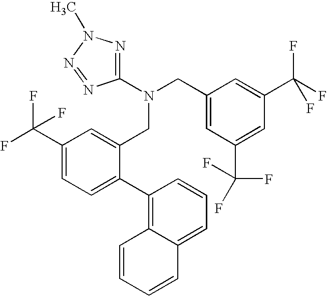

To a solution of naphthalen-1-yl-1-boronic acid (43 mg, 0.27 mmol) in deoxygenated ethanol (0.8 mL) was added the product of preparation 6 (100 mg, 0.18 mmol) in deoxygenated 1,4-dioxane (0.7 mL) followed by Tetrakis(triphenylphosphine)palladium(0) (Pd(PPh₃)₄) (21 mg, 0.018 mmol) in deoxygenated 1,4-dioxane (0.9 mL) and 2 M aqueous Sodium Carbonate (Na₂CO₃) (0.72 mL, 1.44 mmol). The resulting mixture was stirred at 95° C. for 3 hours. The reaction mixture was partitioned between water and ethyl acetate. The organic layer was concentrated and the residue was purified by flash chromatography (Silica gel) (eluted with 9:1 hexane-ethyl acetate) to afford the title compound (54 mg). ¹H NMR (300 MHz, CHLOROFORM-D) δ ppm 4.1 (s, 3H) 4.5 (m, 4H) 7.2 (dd, J=7.2, 1.2 Hz, 1H) 7.4 (m, 3H) 7.5 (m, 4H) 7.7 (m, 3H) 7.9 (t, J=9.2 Hz, 2H); MS (ES⁺) Calc: 609.16. Found: 609.9 (M+1).

For examples 2-60, Analytical HPLC/MS was performed on a Waters 2795 system with Autosampler, UV detection (Waters DAD 996, Waters, Milford, Mass.) monitoring at 215 nm, ELSD detection (SEDEX 75, Sedere, Somerset, N.J.) and mass detection using a Micromass ZQ Spectrometer (Micromass, Manchester, UK). The mobile phase utilized was acetonitrile/water; containing 1% trifluoroacetic acid using a 5 minute gradient 25% to 95% (% acetonitrile) using an Atlantis dC 18 4.6×50 mm, 5 um column (Waters, Milford, Mass.).

### Example 2

### N-[3,5-bis(trifluoromethyl)benzyl]-N-{[4-(trifluoromethyl)biphenyl-2-yl]methyl}-2-methyl-2H-tetrazol-5-amine

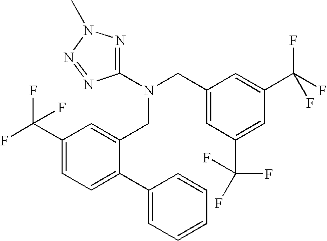

To a solution of phenyl-boronic acid (43 mg, 0.27 mmol) in deoxygenated ethanol (0.8 mL) was added the product of preparation 6 (100 mg, 0.18 mmol) in deoxygenated 1,4-dioxane (0.7 mL) followed by Tetrakis(triphenylphosphine)palladium(0) (Pd(PPh₃)₄) (21 mg, 0.018 mmol) in deoxygenated 1,4-dioxane (0.9 mL) and 2 M aqueous Sodium Carbonate (Na₂CO₃) (0.72 mL, 1.44 mmol). The resulting mixture was shaken at 95° C. for 3 hours. The reaction mixture was concentrated, and partitioned between water and ethyl acetate. The organic layer was concentrated and the residue was purified by preparative HPLC to afford the title compound (9.145 mg). MS (ES⁺) Calc: 589.15. Found: 590.3 (M+1). Ret Time 2.84 min

The compounds listed in Table 1 below were prepared using procedures analogous to those described above for the synthesis of Examples 1 and 2 using the appropriate starting materials, which are available commercially, prepared using preparations well-known to those skilled in the art, or prepared in a manner analogous to routes described above for other intermediates.

TABLE 1

| | | | | MS | Retention |
|---|---|---|---|---|---|
| | | | MS | Found | time |
| Ex. # | Compound Name | Compound Structure | Calc | (M + 1) | (minutes) |
| 3 | N-[3,5-bis(trifluoromethyl) benzyl]-N-{[2′-fluoro-4- (trifluoromethyl)biphenyl- 2-yl]methyl)-2-methyl-2H- tetrazol-5-amine | 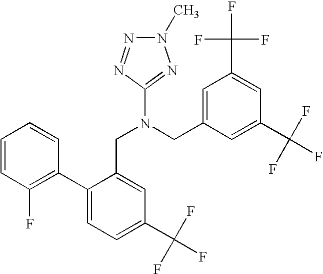 | 577.13 | 578.3 | 2.79 |
| 4 | N-[3,5-bis(trifluoromethyl) benzyl]-N-{[2′,3′-dimethyl- 4-(trifluoromethyl)bipheny- 2-yl]methyl}-2-methyl-2H- tetrazol-5-amine | 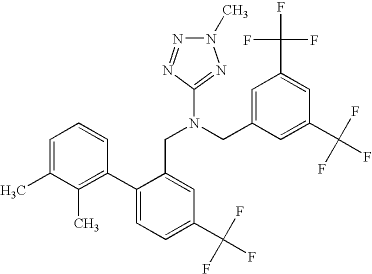 | 587.17 | 588.3 | 3.41 |
| 5 | N-[3,5-bis(trifluoromethyl) benzyl]-N-{[2,5′-dimethyl- 4-(trifluoromethyl) biphenyl-2-yl]methyl}-2- methyl-2H-tetrazol-5-amine | 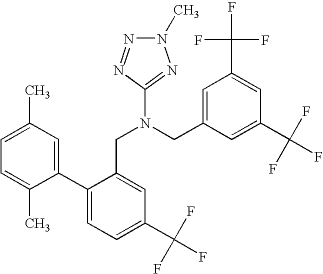 | 587.17 | 588.3 | 3.4 |
| 6 | N-[3,5-bis(trifluoromethyl) benzyl]-N-{[2′-methoxy-4- (trifluoromethyl)biphenyl- 2-yl]methyl}-2-methyl-2H- tetrazol-5-amine | 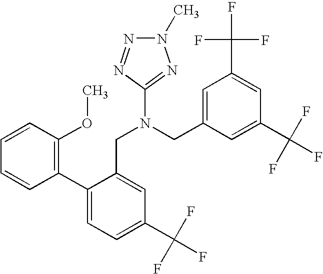 | 589.15 | 590.3 | 2.84 |
| 7 | N-[3,5-bis(trifluoromethyl) benzyl]-N-{[4′-fluoro-2′- methyl-4-(trifluoromethyl) biphenyl-2-yl]methyl}-2- methyl-2H-tetrazol-5-amine | 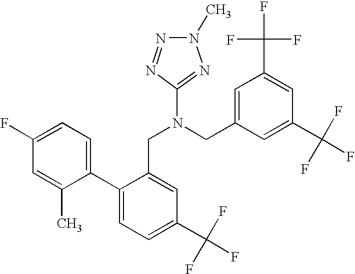 | 591.15 | 592.3 | 3.08 |
| 8 | N-[3,5-bis(trifluoromethyl) benzyl]-N-{[2′4′-difluoro- 4-(trifluoromethyl) biphenyl-2-yl]methyl)-2- methyl-2H-tetrazol-5-amine | 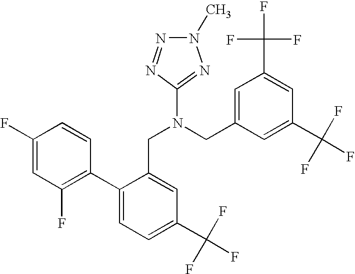 | 595.12 | 596.3 | 2.83 |
| 9 | N-[3,5-bis(trifluoromethyl) benzyl]-N-{[2′,5′-difluoro- 4-(trifluoromethyl) biphenyl-2-yl]methyl)-2- methyl-2H-tetrazol-5-amine | 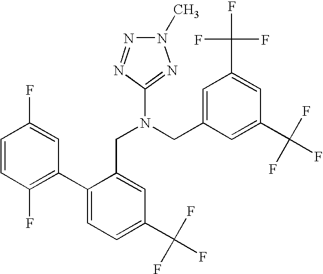 | 595.12 | 596.3 | 2.77 |
| 10 | 1-[2′-{[[3,5-bis (trifluoromethyl)benzyl] (2-methyl-2H-tetrazol-5- yl)amino]methyl]-4′-(tri- fluoromethyl)biphenyl-2-yl] ethanone | 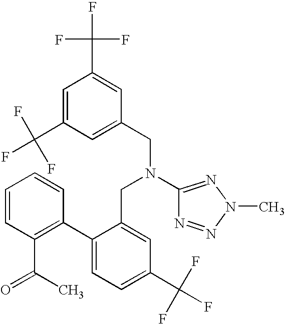 | 601.15 | 602.3 | 2.47 |
| 11 | N-(3,5-bis(trifluoromethyl) benzyl]-N-{[2′-methoxy-5′- methyl-4-(trifluoromethyl) biphenyl-2-yl]methyl}-2- methyl-2H-tetrazol-5-amine | 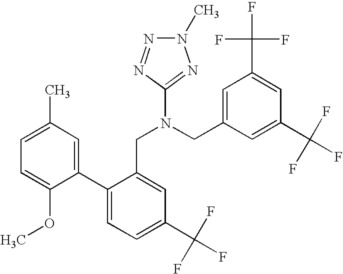 | 603.17 | 604.3 | 3.05 |
| 12 | N-[3,5-bis(trifluoromethyl) benzyl]-N-{[2′-ethoxy-4- (trifluoromethyl)biphenyl- 2-yl]methyl}-2-methyl-2H- tetrazol-5-amine | 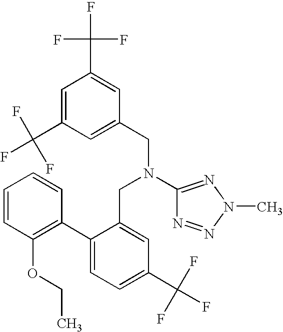 | 603.17 | 604.3 | 3.05 |
| 13 | N-[3,5-bis(trifluoromethyl) benzyl]-N-{(2-(methylthio)- 4-(trifluoromethyl) biphenyl-2-yl [methyl}-2- methyl-2H-tetrazol-5-amine | 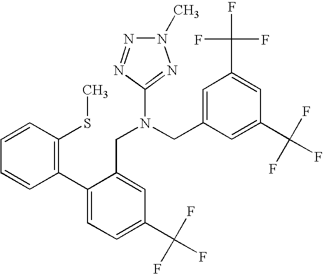 | 605.13 | 606.3 | 2.95 |
| 14 | N-[3,5-bis(trifluoromethyl) benzyl]-N-{[2′-fluoro-3′- methoxy-(trifluoromethyl) biphenyl-2-yl]methyl}-2- methyl-2H-tetrazol-5-amine | 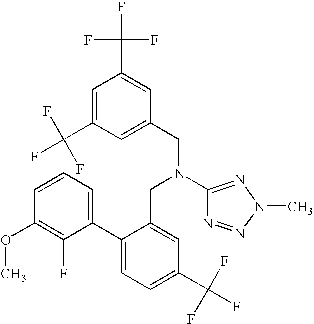 | 607.14 | 608.3 | 2.55 |
| 15 | N-[3,5-bis(trifluoromethyl) benzyl]-N-{[5′-fluoro-2′- methoxy-4- (trifluoromethyl)biphenyl- 2-yl]methyl)-2-methyl-2H- tetrazol-5-amine | 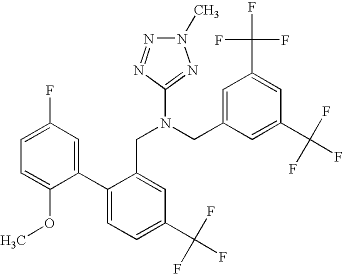 | 607.14 | 608.3 | 2.78 |
| 16 | N-[3,5-bis(trifluoromethyl) benzyl]-N-[2-(1-naphthyl)- 5-(trifluoromethyl)benzyl]- 2-methyl-2H-tetrazol-5- amine | 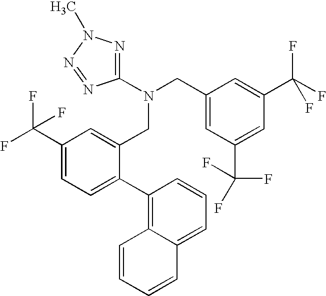 | 609.16 | 610.3 | 3.21 |
| 17 | N-[3,5-bis(trifluoromethyl) benzyl]-N-[2-isoquinolin-5- yl-5-(trifluoromethyl) benzyl]-2-methyl-2H- tetrazol-5-amine | 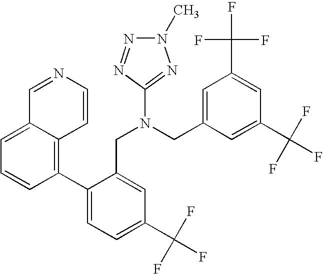 | 610.15 | 611.3 | 1.71 |
| 18 | N-[3,5-bis(trifiuoromethyl) benzyl]-N-{[4′-methoxy-2′, 6′-dimethyl-4-(tri- fluoromethyl)biphenyl-2-yl] methyl}-2-methyl-2H- tetrazol-5-amine | 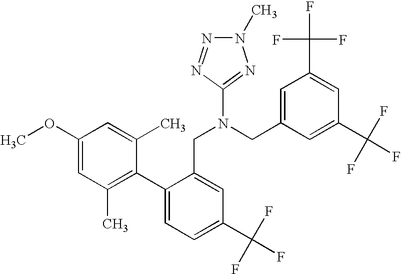 | 617.18 | 618.4 | 3.18 |
| 19 | N-[3,5-bis(trifluoromethyl) benzyl]-N-{[2′,4- dimethoxy-4- (trifluoromethyl)biphenyl- 2-yl]methyl}-2-methyl-2H- tetrazol-5-amine | 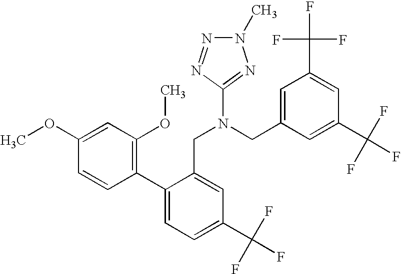 | 619.16 | 620.3 | 2.74 |
| 20 | N-[3,5-bis(trifluoromethyl) benzyl]-N-{[2′,5′- dimethoxy-4- (trifluoromethyl)biphenyl- 2-yl]methyl}-2-methyl-2H- tetrazol-5-amine | 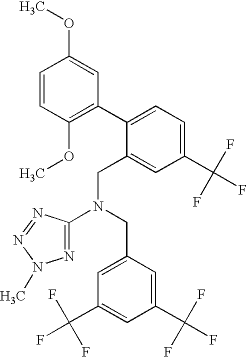 | 619.16 | 620.3 | 2.73 |
| 21 | N-(3,5-bis(trifluoromethyl) benzyl]-N-{[2′,6′- dimethoxy-4- (trifluoromethyl)biphenyl- 2-yl]methyl}-2-methyl-2H- tetrazol-5-amine | 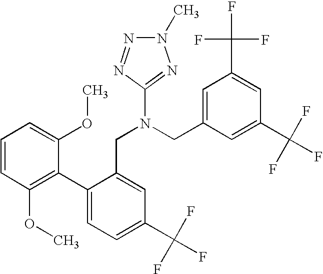 | 619.16 | 620.3 | 2.73 |
| 22 | N-13,5-bis(trifluoromethyl) benzyl]-N-[2-(4-methyl-1- naphthyl)-5- (trifluoromethyl)benzyl]-2- methyl-2H-tetrazol-5-amine | 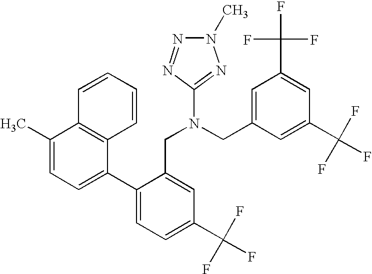 | 623.17 | 624.3 | 3.52 |
| 23 | N-[3,5-bis(trifluoromethyl) benzyl]-N-{[3′-(1H- pyrazol-1-yl)- (trifluoromethyl)biphenyl- 2-yl]methyl}-2-methyl-2H- tetrazol-5-amine | 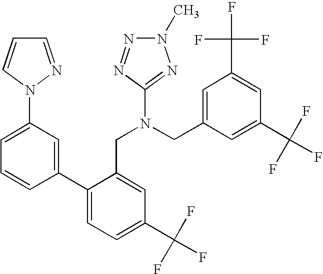 | 625.16 | 626.3 | 2.48 |
| 24 | N-[3,5-bis(trifluoromethyl) benzyl]-N-{[2′,4-bis (trifluoromethyl)biphenyl-2- yl]methyl}-2-methyl-2H- tetrazol-5-amine | 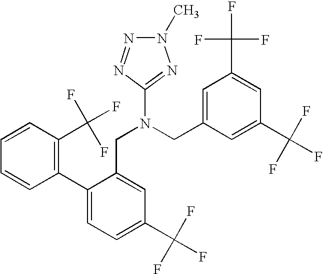 | 627.13 | 628.3 | 3.01 |
| 25 | N-[3,5-bis(trifluoromethyl) benzyl]-N-{[5′-isopropyl-2′- methoxy-4- (trifluoromethyl)biphenyl- 2-yl]methyl}-2-methyl-2H- tetrazol-5-amine | 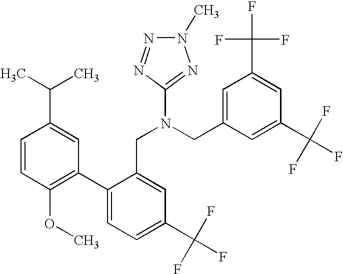 | 631.2 | 632.4 | 3.58 |
| 26 | N-[3,5-bis(trifluoromethyl) benzyl]-N-{[4- (trifluoromethyl)-1,1′:2′, 1″-terphenyl-2-yl]methyl}- 2-methyl-2H-tetrazol-5- amine | 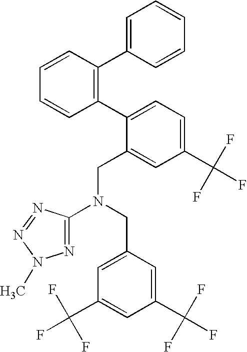 | 635.17 | 636.3 | 3.38 |
| 27 | N-[3,5-bis(trifluoromethyl) benzyl]-N-{[2′- (trifluoromethoxy)-4- (trifluoromethyl)biphenyl- 2-yl]methyl}-2-methyl-2H- tetrazol-5-amine | 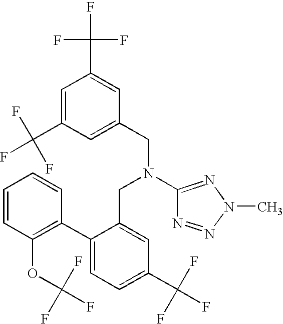 | 643.12 | 644.3 | 3.17 |
| 28 | N-[3,5-bis(trifluoromethyl) benzyl]-N-{[2′-phenoxy-4- (trifluoromethyl)biphenyl- 2-yl]methyl}-2-methyl-2H- tetrazol-5-amine | 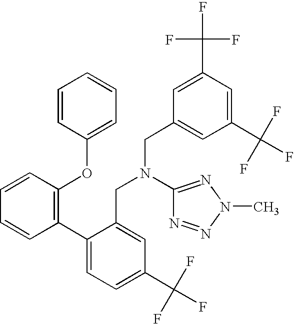 | 651.17 | 652.3 | 3.41 |
| 29 | N-[3,5-bis(trifluoromethyl) benzyl]-N-{[3′-(3,5- dimethyl-1H-pyrazol-1-yl)- 4-(trifluoromethyl) biphenyl-2-yl]methyl}-2- methyl-2H-tetrazol-5-amine | 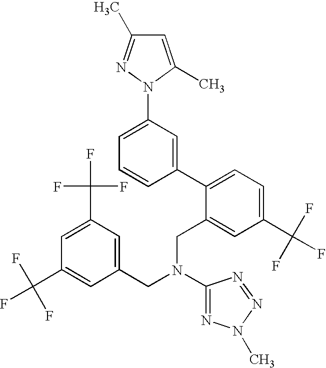 | 653.19 | 654.3 | 2.54 |
| 30 | N-{[2′-(benzyloxy)-4- (tri-fluoromethyl)biphenyl- 2-yl]methyl}-N-[3,5-bis (trifluoromethyl)benzyl]-2- methyl-2H-tetrazol-5-amine | 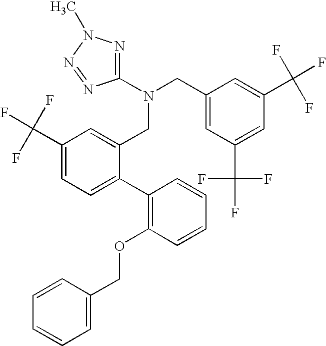 | 665.18 | 666.4 | 3.36 |
| 31 | N-{[4′-(benzyloxy)-2′-fluoro- 4-(trifluoromethyl)biphenyl-2- yl]methyl}-N-[3,5-bis (trifluoromethyl)benzyl]-2- methyl-2H-tetrazol-5-amine | 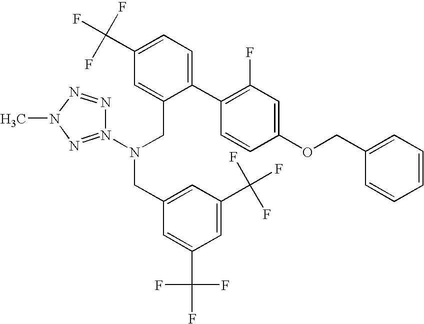 | 683.17 | 684.3 | 3.36 |
| 32 | N-[3,5-bis(trifluoromethy) benzyl]-N-{[3′-methyl-4- (trifluoromethyl)biphenyl- 2-yl]methyl}-2-methyl-2H- tetrazol-5-amine | 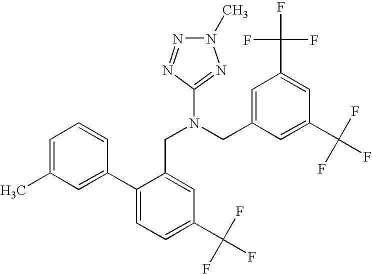 | 573.16 | 574.3 | 3.19 |
| 33 | N-[3,5-bis(trifluoromethyl) benzyl]-N-{[4′-methyl-4- (trifluoromethyl)biphenyl-2- yl]methyl}-2-methyl-2H- tetrazol-5-amine | 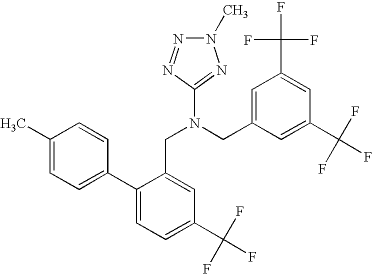 | 573.16 | 574.3 | 3.14 |
| 34 | N-[3,5-bis(trifluoromethyl) benzyl]-N-{[4′-fluoro-4- (trifluoromethyl)biphenyl-2- yl]methyl}-2-methyl-2H- tetrazol-5-amine | 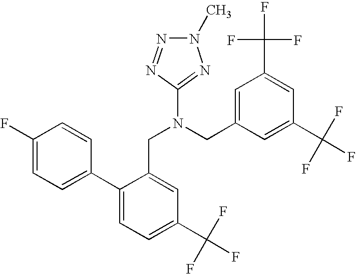 | 577.13 | 578.3 | 2.84 |
| 35 | 2′-{[[3,5-bis (trifluoromethyl)benzyl](2- methyl-2H-tetrazol-5-yl) amino]methyl}-4′- (trifluoromethyl)biphenyl-3- carbonitrile | 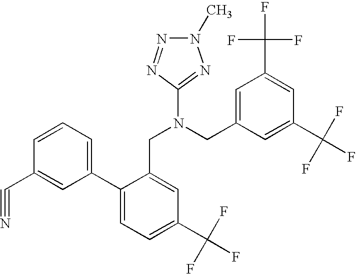 | 584.14 | 585.3 | 2.37 |
| 36 | N-[3,5-bis(trifluoromethyl) benzyl]-N-{[3′,4′-dimethyl- 4-(trifluoromethyl)biphenyl- 2-yl]methyl}-2-methyl-2H- tetrazol-5-amine | 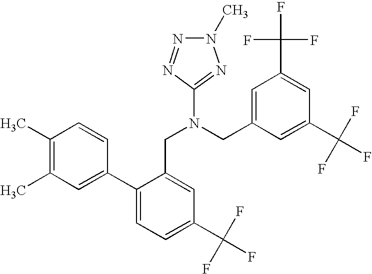 | 587.17 | 588.3 | 3.42 |
| 37 | N-[3,5-bis(trifluoromethyl) benzyl]-N-{[3′-methoxy-4- (trifluoromethyl)biphenyl-2- yl]methyl}-2-methyl-2H- tetrazol-5-amine | 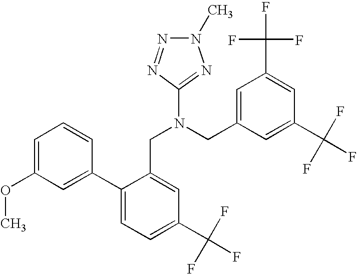 | 589.15 | 590.3 | 2.83 |
| 38 | N-[3,5-bis(trifluoromethyl) benzyl]-N-[2-(2,3-dihydro-1- benzofuran-5-yl)-5- (trifluoromethyl)benzyl]-2- methyl-2H-tetrazol-5-amine | 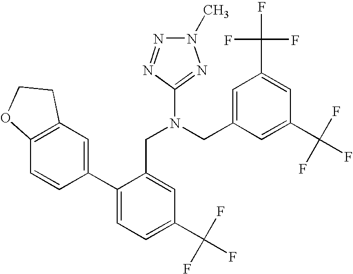 | 601.15 | 602.3 | 2.8 |
| 39 | N-[3,5-bis(trifluoromethyl) benzyl]-N-{[3′-isopropyl-4- (trifluoromethyl)biphenyl-2- yl]methyl}-2-methyl-2H- tetrazol-5-amine | 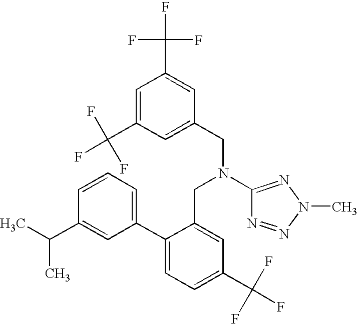 | 601.19 | 602.3 | 3.67 |
| 40 | N-[3,5-bis(trifluoromethyl) benzyl]-N-[2-(2,3-dihydro-1, 4-benzodioxin-6-yl)-5- (trifluoromethyl)benzyl]-2- methyl-2H-tetrazol-5-amine | 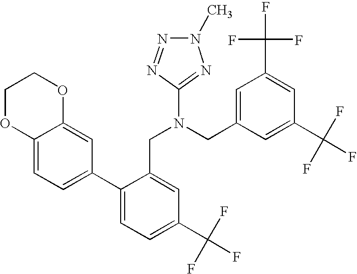 | 617.15 | 618.3 | 2.64 |
| 41 | N-[3,5-bis(trifluoromethyl) benzyl]-N-{[4- (trifluoromethyl)-1,1′:3′,1″- terphenyl-2-yl]methyl}-2- methyl-2H-tetrazol-5-amine | 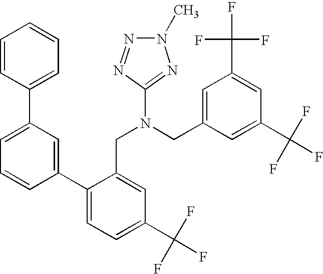 | 635.17 | 636.3 | 1.75 |
| 42 | N-[3,5-bis(trifluoromethyl) benzyl]-N-{(3′- (trifluoromethoxy)-4- (trifluoromethyl)biphenyl-2- yl]methyl}-2-methyl-2H- tetrazol-5-amine | 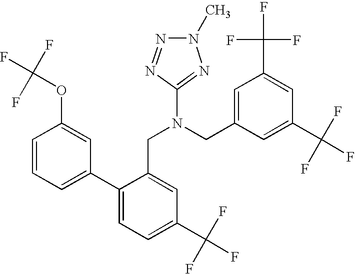 | 643.12 | 644.3 | 3.29 |

### Example 43

### (3,5-Bis-trifluoromethyl-benzyl)-[(5′-chloro-2′-methoxy-4-trifluoromethyl-biphenyl-2-yl)methyl]-(2-methyl-2H-tetrazol-5-yl)-amine

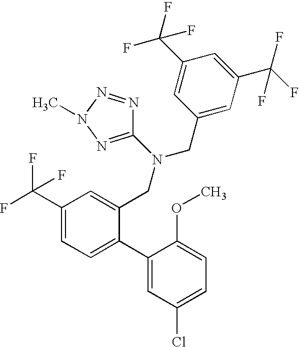

To a solution of the product from preparation 7 (45 mg, 0.075 mmol) in ethanol (0.2 mL) was added 2-bromo-4-chloro-1-methoxybenzene (11 mg, 0.05 mmol) in 1,4-dioxane (0.2 mL) followed by Pd(PPh₃)₄ (10 mg) and 2M aq Na₂CO₃ (0.2 mL, 0.4 mmol). The mixture was stirred at 95° C. for 2 hours, after which the solvent was removed. The residue was partitioned between water (2 ml) and ethyl acetate (twice at 2 ml). The combined organic layers were washed with saturated NaCl (2 ml), dried (magnesium sulfate) and concentrated. The residue was purified by HPLC to yield the title compound (9.7 mg). MS (ES+) Calc: 623.11. Found: 623.9 (M+1).

The compounds listed in Table 2 below were prepared using procedures analogous to those described above for the synthesis of example 43 using the appropriate starting materials which are available commercially, prepared using preparations well-known to those skilled in the art, or prepared in a manner analogous to routes described above for other intermediates.

TABLE 2

| | | | | MS | Retention |
|---|---|---|---|---|---|
| Ex. | | | MS | Found | time |
| # | Compound Name | Compound Structure | Calc | (M + 1) | (minutes) |
| 44 | 2′-{[(3,5-Bis-trifluoromethyl- benzyl)-(2-methyl-2H-tetrazol- 5-yl)-amino]-methyl}-6- methoxy-4′-trifluoromethyl- biphenyl-3-carbonitrile | 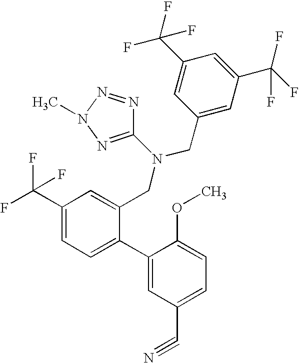 | 614.15 | 614.7 | 2.3 |
| 45 | (2′-{[(3,5-Bis-trifluoromethyl- benzyl)-(2-methyl-2H-tetrazol- 5-yl)-amino]-methyl}-6- methoxy-4′-trifluoromethyl- biphenyl-3-yl)-acetonitrile | 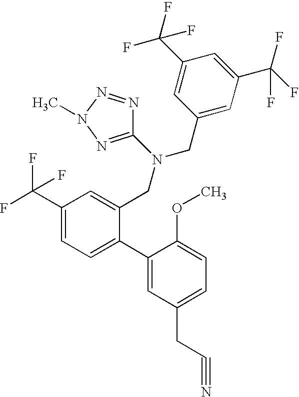 | 628.16 | 628.7 | 2.2 |

### Example 46

### (3,5-Bis-trifluoromethyl-benzyl)-[2-(2-methoxy-5-methyl-phenyl)-6-trifluoromethyl-pyridin-3-ylmethyl]-(2-methyl-2H-tetrazol-5-yl)-amine

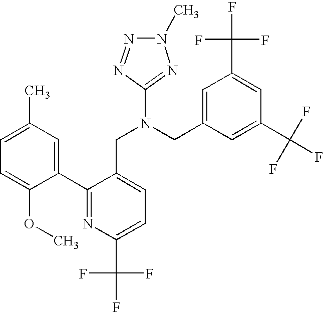

To a solution of 2-methoxy-5-methylphenylboronic acid (0.15 mmol) in ethanol (0.5 mL) was added the product of preparation 10 (0.1 mmol) in 1,4-dioxan (0.4 mL) followed by Pd(PPh₃)₄ in ethanol (0.5 mL) and Na₂CO₃ (0.8 mmol). The resulting mixture was shaken at 95° C. for 16 hours, diluted with ethyl acetate (2 mL) and washed with 10% w/v Na₂CO₃. The mixture was purified by Shimadzu HPLC using nonpolar acidic method to yield the title compound (43.2 mg). ¹H NMR (400 MHz, CHLOROFORM-D) δ ppm 2.3 (s, 3H) 3.7 (s, 3H) 4.2 (s, 3H) 4.38 (d, J=17.0 Hz, 1H) 4.43 (d, J=17.0 Hz, 1H) 4.6 (d, J=15.8 Hz, 1H) 4.8 (d, J=16.8 Hz, 1H) 6.8 (d, J=8.5 Hz, 1H) 7.1 (d, J=2.1 Hz, 1H) 7.2 (dd, J=8.4, 2.2 Hz, 1H) 7.5 (s, 2H) 7.6 (d, J=7.9 Hz, 1H) 7.7 (s, 1H) 7.8 (d, J=7.9 Hz, 1H); MS (ES+) Calc: 604.16. Found: 605.2 (M+1).

The compounds listed in Table 3 below were prepared using procedures analogous to those described above for the synthesis of example 46 using the appropriate starting materials which are available commercially, prepared using preparations well-known to those skilled in the art, or prepared in a manner analogous to routes described above for other intermediates.

TABLE 3

| | | | | MS | Retention |
|---|---|---|---|---|---|
| Ex. | | | MS | Found | time |
| # | Compound Name | Compound Structure | Calc | (M + 1) | (minutes) |
| 47 | (3,5-Bis-trifluoromethyl- benzyl)-[2-(2,5-dimethoxy- phenyl)-6-trifluoromethyl- pyridin-3-ylmethyl]-(2- methyl-2H-tetrazol-5-yl)- amine | 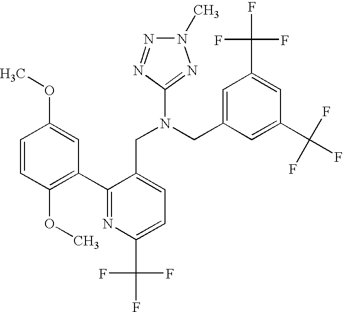 | 620.16 | 621.2 | 2.68 |
| 48 | (3,5-Bis-trifluoromethyl- benzyl)-[2-(5-isopropyl-2- methoxy-phenyl)-6- trifluoromethyl-pyridin-3- ylmethyl]-(2-methyl-2H- tetrazol-5-yl)-amine | 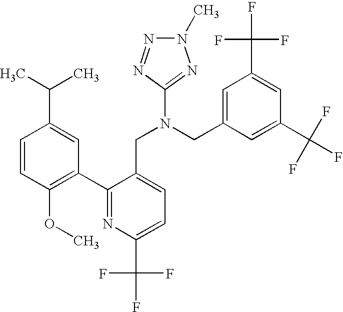 | 632.19 | 633.2 | 3.18 |

### Example 49

### 4-[1-(2-{[(3,5-Bis-trifluoromethyl-benzyl)-(2-methyl-2H-tetrazol-5-yl)-amino]-methyl}-4-trifluoromethyl-phenyl)-propoxy]-benzamide

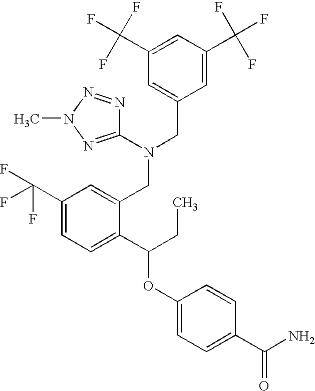

To a solution of the product of preparation 11 (21 mg, 0.038 mmol) and 4-hydroxybenzamide (7.98 mg, 0.058 mmol) in THF (1 mL) was added triphenylphosphine (15 mg, 0.058 mmol) and diisopropylcarbodiimide (24 mg, 0.116 mmol). The mixture was stirred overnight, filtered through Celite, washed with ethyl acetate and blown dry with N₂. The residue was purified on redisep 12 gm column (Silica gel) (eluted with 1:1 hexane-ethyl acetate) to yield an oil. The oil was purified on Shimadzu HPLC to yield the title compound (5.3 mg). ¹H NMR (400 MHz, CHLOROFORM-D) δ ppm 0.9 (t, J=7.4 Hz, 3H) 1.7 (m, 1H) 1.9 (m, 1H) 3.5 (s, 2H) 4.2 (s, 3H) 4.6 (d, J=16.2 Hz, 1H) 4.7 (d, J=15.8 Hz, 1H) 4.8 (d, J=16.0 Hz, 1H) 4.9 (d, J=15.8 Hz, 1H) 5.4 (dd, J=8.1, 4.2 Hz, 1H) 6.78 (d, J=8.92, 2H) 7.3 (s, 1H) 7.5 (d, J=8.1 Hz, 1H) 7.6 (d, J=8.1 Hz, 1H) 7.6 (m, 4H) 7.8 (s, 1H); MS (ES+) Calc: 660.19. Found: 661.2 (M+1).

### Example 50

### (3,5-Bis-trifluoromethyl-benzyl)-[2-(2,5-dimethyl-phenoxy)-6-trifluoromethyl-pyridin-3-ylmethyl]-(2-methyl-2H-tetrazol-5-yl)-amine

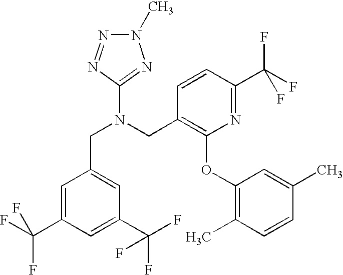

To a mixture of the product from preparation 10 (23.9 mg, 0.0424 mmol) and 2,5-dimethylphenol (10.7 mg, 0.0875 mmol) in DMF (1 mL) was added Cesium Carbonate (69.3 mg, 0.212 mmol). The mixture was heated at 80-96° C. for 2 hours, diluted with ethyl acetate (2 mL) and washed with 10% w/v Na₂CO₃. Solvent was removed and the residue was purified on Shimadzu HPLC to yield the title compound (25.6 mg). ¹H NMR (400 MHz, CHLOROFORM-D) δ ppm 2.0 (s, 3H) 2.3 (s, 3H) 4.2 (s, 3H) 4.8 (s, 2H) 4.9 (s, 2H) 6.8 (s, 1H) 6.9 (d, J=7.5 Hz, 1H) 7.1 (d, J=7.9 Hz, 1H) 7.3 (d, J=7.7 Hz, 1H) 7.7 (s, 2H) 7.8 (m, 2H); MS (ES+) Calc: 604.16. Found: 605.2 (M+1).

### Example 51

### N-(3,5-Bis-trifluoromethyl-benzyl)-N-(5′-isopropyl-2′-methoxy-4-trifluoromethyl-biphenyl-2-ylmethyl)acetamide

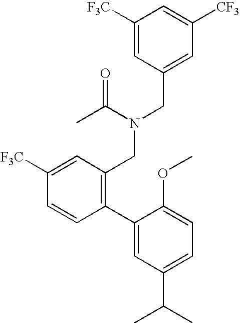

To a solution of (3,5-Bis-trifluoromethyl-benzyl)-5′-isopropyl-2′-methoxy-4-trifluoromethyl-biphenyl-2-ylmethyl)-amine (110 mg, 0.2 mmol) in 3 mL methylene chloride was added Ac₂O (28.3 uL, 30.6 mg, 0.3 mmol), Diisopropylethylamine (56.6 uL, 39 mg, 0.3 mmol) The resulting solution was stirred at room temperature for 4 hours. Solvent was removed in vacuo. The residue was dissolved in methylene chloride (10 mL) and washed with 20 mL of saturated aqueous ammonium chloride, 20 mL of sat. sodium bicarbonate, dried over (sodium sulfate) and concentrated to afford crude product. Purification by chromatography on silica gel (10-20% ethyl acetate in hexane) give the titled compound (106 mg, 90%) as a transparent paste. ¹H NMR (400 MHz, CHLOROFORM-D) δ ppm 1.14, 1.21 (d, J=6.7 Hz, 3H) 1.15, 1.22 (J=6.7 Hz, 3H) 2.01, 2.09 (s, 3H) 2.9 (m, 1H) 3.61, 3.69 (s, 3H) 4.2, 4.32 (d, J=117.2 Hz, 1H) 4.5 (d, J=4.1 Hz, 1H) 4.5 (d, J=6.6 Hz, 1H) 4.7 (m, 1H) 6.79-6.92 (m, 2H) 7.25-7.36 (m, 3H) 7.58-7.61 (m, 3H) 7.7 (m, 1H); MS (ES⁺) Calc: 591.18. Found: 591.9 (M+1).

### Example 52

### Preparation of (3,5-Bis-trifluoromethyl-benzyl)-(5′-isopropyl-2′-methoxy-4-trifluoromethyl-biphenyl-2-ylmethyl)-carbamic acid methyl ester

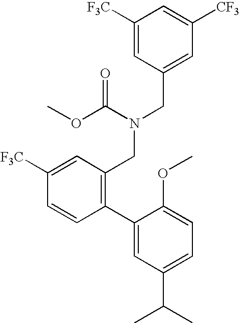

To a solution of (3,5-Bis-trifluoromethyl-benzyl)-(5′-isopropyl-2′-methoxy-4-trifluoromethyl-biphenyl-2-ylmethyl)-amine (110 mg, 0.2 mmol) and Diisopropylethylamine (51.7 mg, 0.4 mmol) in methylene chloride (2 mL) at room temperature was added methyl chloroformate (28.3 mg, 0.3 mmol). The resulting mixture was stirred at room temperature overnight. Solvent was removed under reduced pressure and the residue was dissolved in methylene chloride (20 mL). The solution was washed with 40 mL of saturated aqueous ammonium chloride, 40 mL of saturated sodium bicarbonate, dried over sodium sulfate, and concentrated to afford crude product. Purification by chromatography on silica gel (10% ethyl acetate in hexane) gave the titled compound (85 mg, 70%) as a transparent paste. ¹H NMR (400 MHz, CHLOROFORM-D) δ ppm 1.2 (m, 6H) 2.9 (m, 1H) 3.72 (s, 3H) 3.72, 3.76 (s, 3H) 4.3 (m, 4H) 6.9 (d, J=7.9 Hz, 1H) 6.9 (d, J=2.3 Hz, 1H) 7.2 (s, 1H) 7.3 (d, J=7.9 Hz, 2H) 7.4 (s, 2H) 7.6 (d, J=8.1 Hz, 1H) 7.7 (s, 1H); MS (ES⁺) Calc: 607.18. Found: 608.0 (M+1).

### Example 53

### Preparation of (3,5-Bis-trifluoromethyl-benzyl)-(5′-isopropyl-2′-methoxy-biphenyl-2-ylmethyl)-(2-methyl-2H-tetrazol-5-yl)-amine

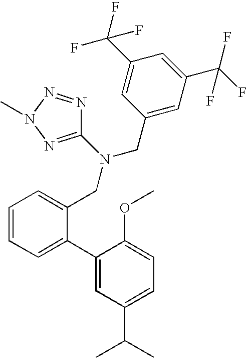

To a solution of (3,5-Bis-trifluoromethyl-benzyl)-(2-bromo-benzyl)-(2-methyl-2H-tetrazol-5-yl)-amine (100 mg, 0.2 mmol) in deoxygenated 1,4-dioxane (1 mL) was added 5-isopropyl-2-methoxyphenylboronic acid (58.8 mg, 0.3 mmol) in deoxygenated ethanol (1 mL) followed by Pd(PPh₃)₄ (23 mg, 0.02 mmol) and 2 M aq. Na₂CO₃ (0.8 mL, 1.6 mmol). The resulting mixture was stirred at 95° C. for 16 hours. The reaction mixture was partitioned between water and ethyl acetate. The organic layer was concentrated and the residue was purified by flash chromatography (Silica gel) (eluted with 3:1 hexane-ethyl acetate) to afford the title compound (54 mg). ¹H NMR (300 MHz, CHLOROFORM-D) δ ppm 1.1 (d, J=6.8 Hz, 6H) 2.7 (m, 1H) 3.6 (s, 3H) 4.0 (s, 3H) 4.3 (s, 2H) 4.5 (d, J=10.3 Hz, 2H) 6.7 (d, J=8.4 Hz, 1H) 6.8 (d, J=2.3 Hz, 1H) 7.0 (dd, J=8.3, 2.3 Hz, 1H) 7.2 (m, 4H) 7.3 (s, 2H) 7.6 (s, 1H);

MS (ES⁺) Calc: 563.21. Found: 563.9 (M+1).

The compounds listed in Table 4 below were prepared using procedures analogous to those described above for the synthesis of example 53 using the appropriate starting materials which are available commercially, prepared using preparations well-known to those skilled in the art, or prepared in a manner analogous to routes described above.

TABLE 4

| | | | | MS | Retention |
|---|---|---|---|---|---|
| Ex. | | | MS | Found | time |
| # | Compound Name | Compound Structure | Calc | (M + 1) | (minutes) |
| 54 | N-[3,5- bis(trifluoromethyl)benzyl]- N-{[2′-chloro-5′-methyl-4- (trifluoromethyl)biphenyl- 2-yl]methyl}-2-methyl-2H- tetrazol-5-amine | 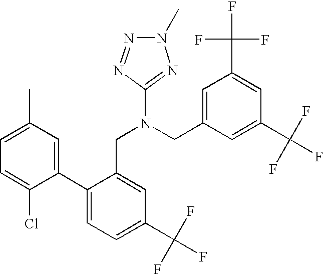 | 607.12 | 608.13 | 2.54 |
| 55 | N-[3,5- bis(trifluoromethyl)benzyl]- N-{[2′-fluoro-5′-methyl-4- (trifluoromethyl)biphenyl- 2-yl]methyl}-2-methyl-2H- tetrazol-5-amine | 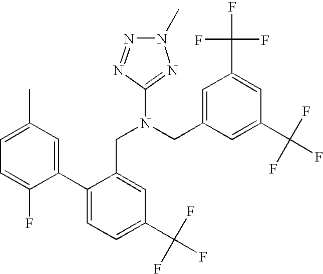 | 591.15 | 592.16 | 2.42 |
| 56 | 1-[2′-{[[3,5- bis(trifluoromethyl)benzyl] (2-methyl-2H-tetrazol-5- yl)amino]methyl}-6-fluoro-4′- (trifluoromethyl)biphenyl- 3-yl]ethanone | 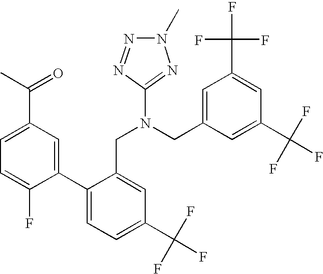 | 619.14 | 620.15 | 2.08 |

### Example 57

### 2′-{[[3,5-bis(trifluoromethyl)benzyl](2-methyl-2H-tetrazol-5-yl)amino]methyl}-6-methoxy-4′-(trifluoromethyl)biphenyl-3-carbaldehyde

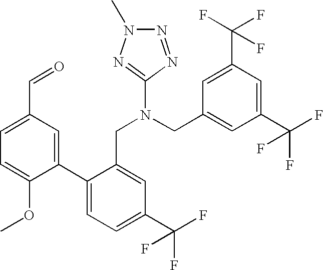

The title compound was prepared using procedures analogous to those described above for the synthesis of example 1 using 3-formyl-4-methoxyphenylboronic acid as starting material. Ms (es+) calc: 617.48. Found: 618.30 (m+1); ret time 1.50.

### Example 58

### N-[3,5-bis(trifluoromethyl)benzyl]-N-{[2′-methoxy-5′-4-[(4-methylpiperazin-1-yl)methyl]-4-trifluoromethyl)biphenyl-2-yl]methyl}-2-methyl-2Htetrazol-5-amine

To a solution of the product from Example 57 (27 mg, 44 umol) in methylene chloride (1 ml) was

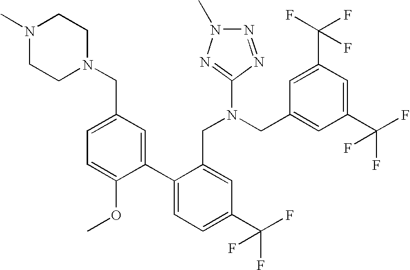

added 4-methylpiperazine (5.3 ul, 48 umol), followed by sodium triacetoxyborohydride (19 mg, 90 umol). The resulting mixture was stirred at room temperature for 16 hours. The reaction mixture was partitioned between 2M NaOH and methylene chloride. The organic layer was concentrated and the residue was purified on Shimadzu HPLC to yield the title compound. MS (ES⁺) Calc: 701.64. Found: 702.00 (M+1) Ret time 1.90.

The compounds listed in Table 5 below were prepared using procedures analogous to those described above for the synthesis of example 58 using the appropriate starting materials which are available commercially, prepared using preparations well-known to those skilled in the art, or prepared in a manner analogous to routes described above.

TABLE 5

| | | | | MS | Retention |
|---|---|---|---|---|---|
| Ex. | | | MS | Found | time |
| # | Compound Name | Compound Structure | Calc | (M + 1) | (minutes) |
| 59 | N-[3,5- bis(trifluoromethyl)benzyl]-N- {[5dimethylamino)methyl}- 2′-methoxy-4- (trifluoromethyl)biphenyl- 2-yl]methyl}-2-methyl-2H- tetrazol-5-amine | 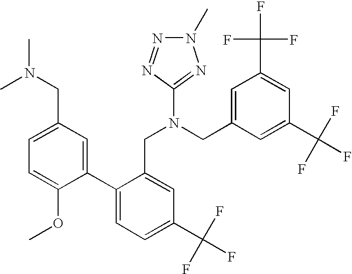 | 646.56 | 646.90 | 1.80 |
| 60 | Methyl N-{[2′-{[[3,5- bis(trifluoromethyl)benzyl] (2-methyl-2H-tetrazol-5- yl)amino]methyl}-6- methoxy- 4′(trifluoromethyl)biphenyl- 3-yl]methyl}-n- methylglycinate | 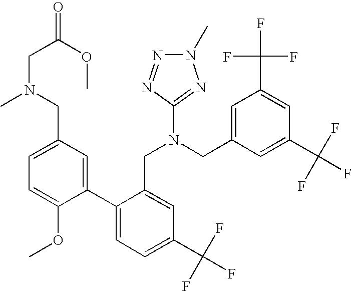 | 704.60 | 704.90 | 2.00 |

### Preparation 18

### 2-{[(3,5-Bis-trifluoromethyl-benzyl-(2-methyl-2H-tetrazol-5-yl)-amino]-methyl}-4-trifluoromethyl-benzonitrile

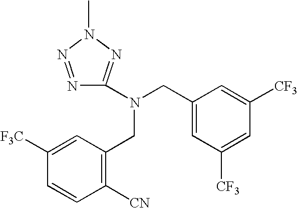

To a solution of (3,5-bis-trifluoromethyl-benzyl)-(2-bromo-5-trifluoromethyl-benzyl)-(2-methyl-2H-tetrazol-5-yl)amine (4.0 g, 7.11 mmol) in DMF (0.5 mL) was added copper(l) cyanide (0.76 g, 8.54 mmol). The mixture was heated at 170° C. for 12 hours. The reaction was cooled to room temperature and quenched with ammonium chloride. Ethyl acetate was added. The organic layer was filtered through magnesium sulfate and evaporated. The residue was purified by flash chromatography to yield the title compound (3.2 g). ¹H NMR (300 MHz, CHLOROFORM-D) □ 4.2 (s, 3H) 4.9 (s, 2H) 4.95 (s, 2H) 7.6 (d, 1H) 7.65 (s, 2H) 7.70 (s, 1H) 7.75 (s, 1H). MS (ES+) Calc: 508.1. Found: 509.1 (M+1).

### Preparation 19

### 1-(2{[(3,5-Bis-trifluoromethyl-benzyl(2-methyl-2H-tetrazol-5-yl)-amino]-methyl}-4-trifluoromethyl-phenyl)-butan-1-one

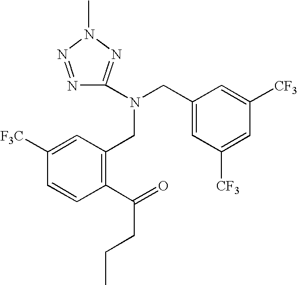

To a solution of 2-([(3,5-bis-trifluoromethyl-benzyl)-(2-methyl-2H-tetrazol-5-yl)-amino]-methyl-4-trifluoromethyl-benzonitrile (1.0 g, 1.96 mmol) in toluene (18 mL) was added n-propylMgBr (2.0 M in diethyl ether, 2.16 mL, 4.33 mmol). The mixture was heated at 60° C. for 0.5 hour in a microwave. The reaction was quenched with 1 N hydrochloric acid and stirred for 0.5 hour. Ethyl acetate was added. The organic layer was filtered through magnesium sulfate and evaporated to provide an oil that was purified by silica gel chromatography to yield the title compound (0.79 g). ¹H NMR (300 MHz, CHLOROFORM-D) δ ppm 0.95 (t, 3H), 1.70 (m, 2H) 2.85 (t, 2H) 42 (s, 3H) 4.9 (s, 2H) 4.95 (s, 2H) 7.59 (s, 1H) 7.60 (d, 1H) 7.70 (s, 2H) 7.75 (s, 1H). MS (ES+) Calc: 553.4. Found: 554 (M+1).

### Preparation 20

### 1-(2-{[(3,5-Bis-trifluoromethyl-benzyl)-2-methyl-2H-tetrazol-5-yl)amino]-methyl}-4-trifluoromethyl-phenyl)-3-dimethylamino-2-ethyl-prop-2-en-1-one

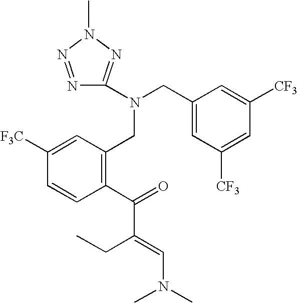

Dimethylformamide-dimethylacetal (5 mL) was added to 1-(2-{[(3,5-bis-trifluoromethyl-benzyl)-(2-methyl-2H-tetrazol-5-yl)-amino]-methyl}-4-trifluoromethyl-phenyl)-butan-1-one (0.50 g, 0.903 mmol). The mixture was heated at 110° C. for 12 hours. The reaction was brought to room temperature and concentrated in vacuo to provide an oil that was used without further purification (0.62 g). ¹H NMR (300 MHz, CHLOROFORM-D) δ ppm 0.95 (t, 3H), 2.2 (s, 6H) 2.60 (m, 2H) 4.2 (s, 3H) 4.9 (s, 2H) 4.95 (s, 2H) 7.25 (s, 1H) 7.45 (s, 1H) 7.55 (s, 1H) 7.75 (s, 2H) 8.00 (d, 1H); MS (ES+) Calc: 608.1. Found: 609 (M+1).

### Preparation 21

### 2-[3-(2-{[(3,5-Bis-trifluoromethyl-benzyl)-(2-methyl-2H-tetrazol-5-yl)-amino]-methyl}-4-trifluoromethyl-phenyl)-4-ethyl-pyrazol-1-yl]-ethanol

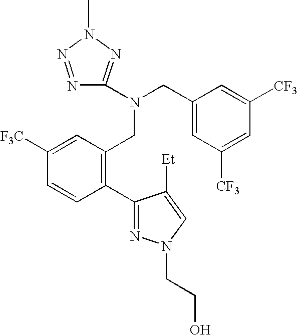

To a solution of 1-(2-{[(3,5-bis-trifluoromethyl-benzyl)-(2-methyl-2H-tetrazol-5-yl)-amino]-methyl}-4-trifluoromethyl-phenyl)-3-dimethylamino-2-ethyl-prop-2-en-1-one (0.62 g, 1.03 mmol) in ethanol (5 mL) was added 2-hydroxyethylhydrazine (0.12 g, 1.54 mmol). The mixture was heated at 95° C. for 3.5 hours. The reaction was brought to room temperature and concentrated in vacuo to provide a crude oil that was purified by silica get chromatography to yield the title compound (0.19 g). ¹H NMR (300 MHz, CHLOROFORM-D) δ ppm 1.02 (t, 3H), 2.3 (m, 2H) 4.0 (m, 2H) 4.2 (s, 3H) 4.25 (m, 2H) 4.6 (s, 3H) 4.95 (s, 2H) 7.30 (s, 1H) 7.45 (s, 1H) 7.55 (s, 1H) 7.59 (s, 1H) 7.79 (s, 1H); MS (ES+) Calc: 621.1. Found: 622 (M+1).

### Preparation 22

### Ethyl{3-[2-{[[3,5-bis(trifluoromethyl)benzyl](2-methyl-2H-tetrazol-5-yl)amino]methyl}-4-(trifluoromethyl)phenyl]-4-ethyl-1H-pyrazol-1-yl}acetate

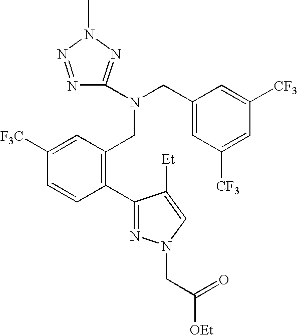

To a solution of N-[3,5-bis(trifluoromethyl)benzyl]-2-methyl-N-[2-(4-ethyl-1H-pyrazol-3-yl)-5-(trifluoromethyl)benzyl]-2H-tetrazol-5-amine (0.025 g, 0.04 mmol) in 0.5 mL DMF was added sodium hydride (0.008 g, 0.21 mmol) and the mixture was allowed to stir at room temperature for 15 minutes. The mixture was heated at 65° C. for 0.5 hours and then cooled to room temperature. Ethyl bromoacetate (0.036 g, 0.21 mmol) and the reaction was heated to 65° C. for 12 hours. The reaction was quenched with water. Ethyl acetate was added. The organic layer was filtered through magnesium sulfate and evaporated to provide an oil that was purified by silica gel chromatography to yield the title compound (0.028 g).

¹H NMR (300 MHz, CHLOROFORM-D) δ ppm 1.02 (t, 3H), 1.20 (t, 3H) 2.4 (m, 2H) 4.2 (s, 3H) 4.25 (m, 2H) 4.6 (s, 2H) 4.85 (s, 2H) 4.95 (s, 2H) 7.30 (s, 1H) 7.45 (d, 1H) 7.60 (d, 1H) 7.59 (s, 2H) 7.75 (d, 2H); MS (ES+) Calc 663.1. Found: 664.4 (M+1).

### Preparation 23

### 1-(2-{[(3,5-Bis-trifluoromethyl-benzyl)-(2-methyl-2H-tetrazol-5-yl)-amino]-methyl}-4-trifluoromethyl-phenyl)-2-bromo-butan-1-one

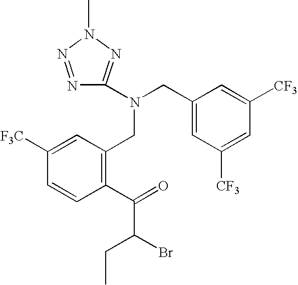

To a solution of 1-(2-([(3,5-Bis-trifluoromethyl-benzyl)-(2-methyl-2H-tetrazol-5-yl)-amino]-methyl)-4-trifluoromethyl-phenyl)butan-1-one (1.08 g, 1.95 mmol) in 12 mL of chloroform was added to a refluxing solution of CuBr₂ (0.88 g, 3.90 mmol) in 20 mL of ethyl acetate. The mixture was refluxed for 2 hours and then cooled to room temperature. The reaction mixture was filtered through Celite®, concentrated and purified on silica gel chromatography to provide 1.23 g of the title compound.

¹H NMR (300 MHz, CHLOROFORM-D) δ ppm 1.03 (t, 3H), 2.2 (m, 2H) 4.2 (s, 3H) 4.85 (m, 5H) 7.45 (s, 1H) 7.5 (d, 1H) 7.65 (d, 2H) 7.7 (s, 2H). MS (ES⁺) Calc. Found: (M+1).

### Example 61

### N-[3,5-bis(trifluoromethyl)benzyl]-N-[2-[5-ethyl-2-methoxymethyl)-1,3-thiazol-4-yl]-5-(trifluoromethyl)benzyl]-2-methyl-2H-tetrazol-5-amine

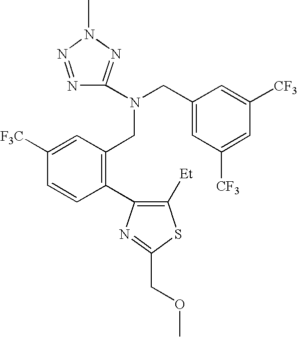

1-(2-{[(3,5-Bis-trifluoromethyl-benzyl)-(2-methyl-2H-tetrazol-5-yl)-amino]-methyl}trifluoromethyl-phenyl)-2-bromo-butan-1-one (15.1 mg, 0.023 mmol) and 2-Methoxy-thioacetamide (3.76 mg, 0.035 mmol) were dissolved in 0.05 mL of ethanol. The reaction mixture was heated to 90° C. for 12 hours. The reaction was concentrated and purified on prep TLC to yield the title compound.

¹H NMR (300 MHz, CHLOROFORM-D) δ ppm 1.1 (t, 3H), 2.6 (q, 2H) 3.4 (s, 3H) 4.1 (s, 3H) 4.5 (s, 2H) 4.6 (s, 2H) 4.7 (s, 2H) 7.30 (d, 1H) 7.5 (m, 4H) 7.70 (s, 1H)

MS (ES+) Calc 638.5. Found: 639.3 (M+1).

The compounds listed in Table 6 below were prepared using procedures analogous to those described above for the synthesis of example 61 using the appropriate starting materials which are available commercially, prepared using preparations well-known to those skilled in the art, or prepared in a manner analogous to routes described above.

TABLE 6

| | | | MS | |
|---|---|---|---|---|
| | | | Calc | MS |
| Ex. | Compound Name | Compound Structure | (ES+) | Found |
| 62 | 2-[2′-{[[3,5- Bis(trifluoromethyl)benzyl](2-methyl- 2H-tetrazol-5-yl)amino]methyl}-6- methyl-4′-(trifluoromethyl)biphenyl- 3-yl]propan-2-ol | 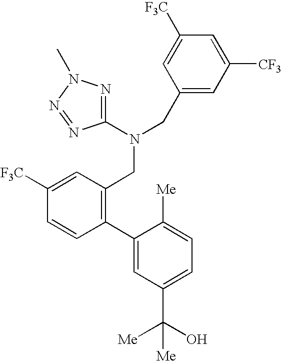 | 631.5 | 614.1 (M − 17) |
| 63 | (R, S) 2-[2′-{[[3,5- Bis(trifluoromethyl)benzyl](2-methyl- 2H-tetrazol-5-yl)amino]methyl}-6- methyl-4′-(trifluoromethyl)biphenyl- 3-yl]butan-2-ol | 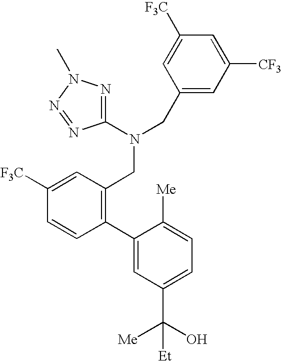 | 645.5 | 628.2 (M − 17) |
| 64 | (R, S) 2-[2′-{[[3,5- Bis(trifluoromethyl)benzyl](2-methyl- 2H-tetrazol-5-yl)amino]methyl}-6- methyl-4′-(trifluoromethyl)biphenyl- 3-yl]-3-methylbutan-2-ol | 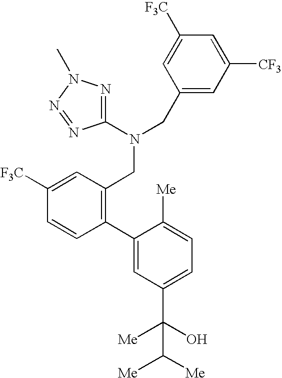 | 659.5 | 642.2 (M − 17) |
| 65 | (R, S) 2-[2′-{[[3,5- Bis(trifluoromethyl)benzyl](2-methyl- 2H-tetrazol-5-yl)amino]methyl}-6- methyl-4′-(trifluoromethyl)biphenyl- 3-yl]pentan-2-ol | 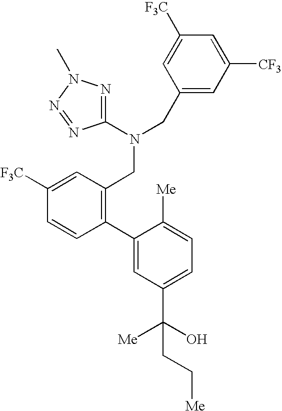 | 659.5 | 642.2 (M − 17) |
| 66 | N-[3,5-bis(trifluoromethyl)benzyl]-2- methyl-N-[2-(1H-pyrazol-3-yl)-5- (trifluoromethyl)benzyl]-2H-tetrazol- 5-amine | 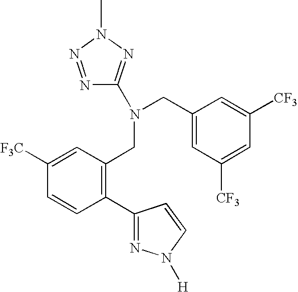 | 549.0 | 550.0 (M − 17) |
| 67 | N-[3,5-bis(trifluoromethyl)benzyl]-N- [2-(1-tert-butyl-4-methyl-1H-pyrazol- 3-yl)-5-(trifluoromethyl)benzyl]-2- methyl-2H-tetrazol-5-amine | 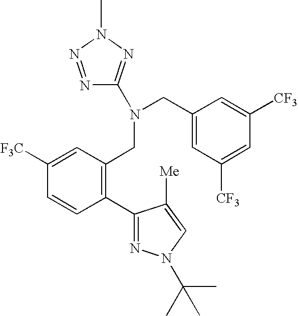 | 619.3 | 620.3 (M − 17) |
| 68 | N-[3,5-bis(trifluoromethyl)benzyl]-N- [2-(1-ethyl-4-methyl-1H-pyrazol-3- yl)-5-(trifluoromethyl)benzyl]-2- methyl-2H-tetrazol-5-amine | 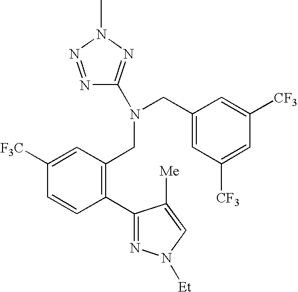 | 591.3 | 592.3 (M − 17) |
| 69 | N-[3,5-bis(trifluoromethyl)benzyl]-2- methyl-N-[2-(4-methyl-1H-pyrazol-3- yl)-5-(trifluoromethyl)benzyl]-2H- tetrazol-5-amine | 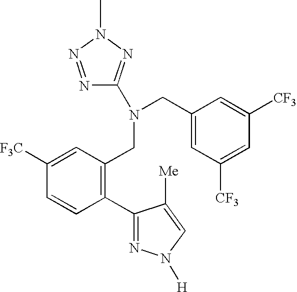 | 563.1 | 564 (M − 17) |
| 70 | N-[3,5-bis(trifluoromethyl)benzyl]-2- methyl-N-[2-(1-methyl-1H-pyrazol-3- yl)-5-(trifluoromethyl)benzyl]-2H- tetrazol-5-amine | 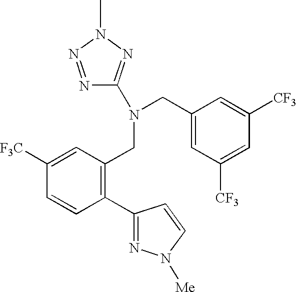 | 563 | 564.1 (M − 17) |
| 71 | 2-{3-[2-{[[3,5- Bis(trifluoromethyl)benzyl](2-methyl- 2H-tetrazol-5-yl)amino]methyl}-4- (trifluoromethyl)phenyl]-4-methyl- 1H-pyrazol-1-yl}ethanol | 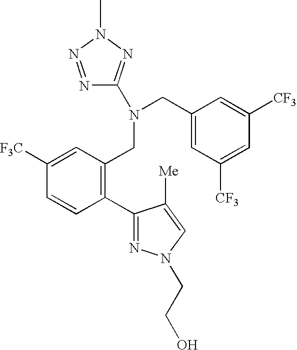 | 607.1 | 608.3 (M − 17) |
| 72 | N-[3,5-bis(trifluoromethyl)benzyl]-N- [2-(1,4-dimethyl-1H-pyrazol-3-yl)-5- (trifluoromethyl)benzyl]-2-methyl- 2H-tetrazol-5-amine | 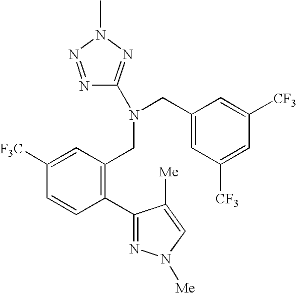 | 577 | 578.1 (M − 17) |
| 73 | N-[3,5-bis(trifluoromethyl)benzyl]-N- [2-(4-isopropyl-1H-pyrazol-3-yl)-5- (trifluoromethyl)benzyl]-2-methyl- 2H-tetrazol-5-amine | 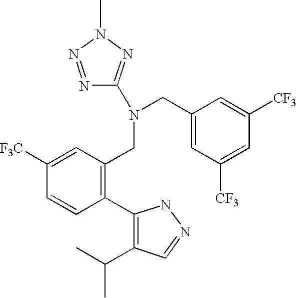 | 591 | 592.3 (M − 17) |
| 74 | N-[3,5-bis(trifluoromethyl)benzyl]-N- [2-(4-isopropyl-1-methyl-1H-pyrazol- 3-yl)-5-(trifluoromethyl)benzyl]-2- methyl-2H-tetrazol-5-amine | 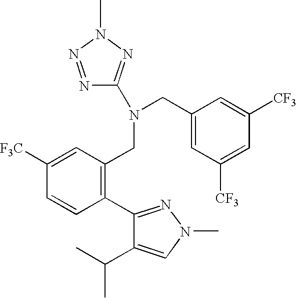 | 605.3 | 606.3 (M − 17) |
| 75 | N-[3,5-bis(trifluoromethyl)benzyl]-2- methyl-N-[2-(4-methyl-1-phenyl-1H- pyrazol-3-yl)-5- (trifluoromethyl)benzyl]-2H-tetrazol- 5-amine | 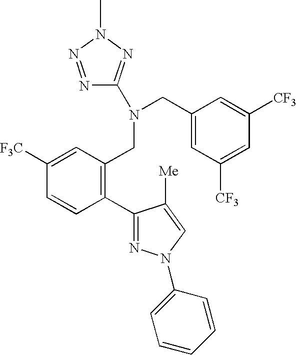 | 639.0 | 640.2 (M + 1) |
| 76 | N-[3,5-bis(trifluoromethyl)benzyl]-N- [2-(1,4-diethyl-1H-pyrazol-3-yl)-5- (trifluoromethyl)benzyl]-2-methyl- 2H-tetrazol-5-amine | 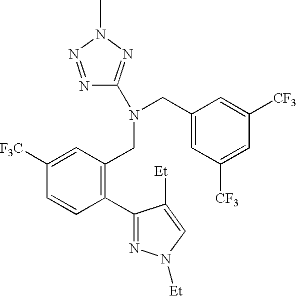 | 605.0 | 606.3 (M + 1) |
| 77 | 2-{3-[2-{[[3,5- Bis(trifluoromethyl)benzyl](2-methyl- 2H-tetrazol-5-yl)amino]methyl}-4- (trifluoromethyl)phenyl]-4-ethyl-1H- pyrazol-1-yl}ethanol | 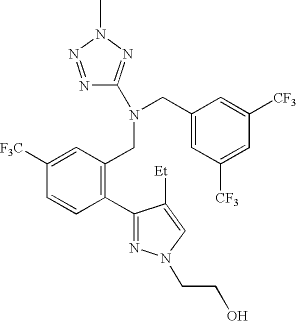 | 621.0 | 622.2 (M + 1) |
| 78 | N-[3,5-bis(trifluoromethyl)benzyl]-N- [2-{1-[2-(dimethylamino)ethyl]-4- ethyl-1H-pyrazol-3-yl}-5- (trifluoromethyl)benzyl]-2-methyl- 2H-tetrazol-5-amine | 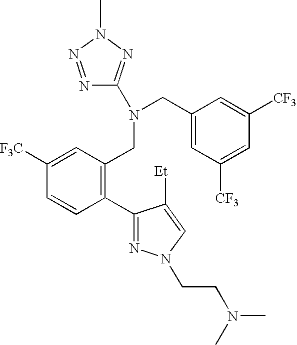 | 649.3 | 650.3 (M + 1) |
| 79 | Ethyl {3-[2-{[[3,5- bis(trifluoromethyl)benzyl](2-methyl- 2H-tetrazol-5-yl)amino]methyl}-4- (trifluoromethyl)phenyl]-4-ethyl-1H- pyrazol-1-yl}acetate | 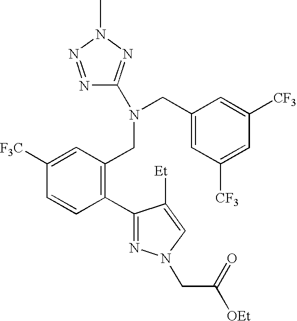 | 663.3 | 664.3 (M + 1) |
| 80 | Methyl 3-{3-[2-{[[3,5- bis(trifluoromethyl)benzyl](2-methyl- 2H-tetrazol-5-yl)amino]methyl}-4- (trifluoromethyl)phenyl]-4-ethyl-1H- pyrazol-1-yl}propanoate | 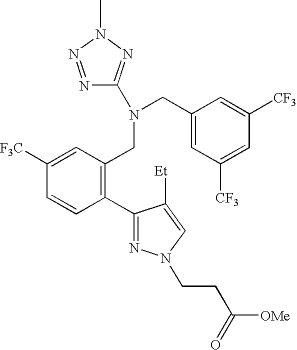 | 663.4 | 664.4 (M + 1) |
| 81 | {3-[2-{[[3,5- Bis(trifluoromethyl)benzyl](2-methyl- 2H-tetrazol-5-yl)amino]methyl}-4- (trifluoromethyl)phenyl]-4-ethyl-1H- pyrazol-1-yl}acetic acid | 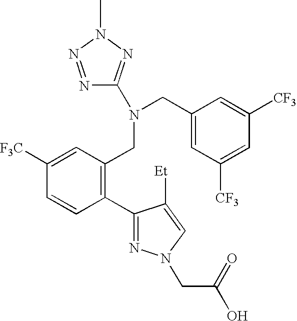 | 633.3 | 634.3 (M + 1) |
| 82 | 3-{3-[2-{[[3,5- Bis(trifluoromethyl)benzyl](2-methyl- 2H-tetrazol-5-yl)amino]methyl}-4- (trifluoromethyl)phenyl]-4-ethyl-1H- pyrazol-1-yl}propanoic acid | 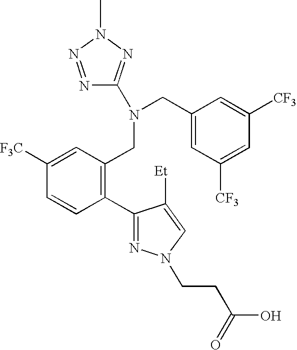 | 649.2 | 650.2 (M + 1) |
| 83 | N-[3,5-bis(trifluoromethyl)benzyl]-N- [2-[4-ethyl-1-(2-methoxyethyl)-1H- pyrazol-3-yl]-5- (trifluoromethyl)benzyl]-2-methyl- 2H-tetrazol-5-amine | 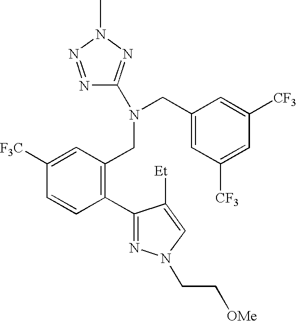 | 635.4 | 636.4 (M + 1) |
| 84 | 3-{3-[2-{[[3,5- Bis(trifluoromethyl)benzyl](2-methyl- 2H-tetrazol-5-yl)amino]methyl}-4- (trifluoromethyl)phenyl]-4-ethyl-1H- pyrazol-1-yl}propanamide | 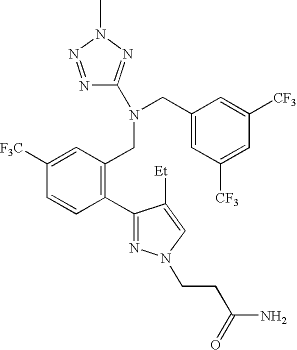 | 648.3 | 649.3 (M + 1) |
| 85 | 2-{3-[2-{[[3,5- Bis(trifluoromethyl)benzyl](2-methyl- 2H-tetrazol-5-yl)amino]methyl}-4- (trifluoromethyl)phenyl]-4-ethyl-1H- pyrazol-1-yl}acetamide | 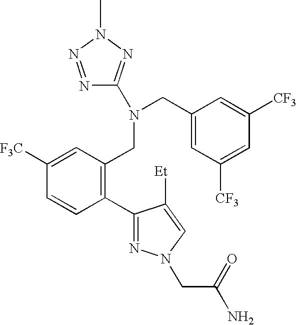 | 634.2 | 636.4 (M + 1) |
| 86 | N-[3,5-bis(trifluoromethyl)benzyl]-N- [2-[4-ethyl-1-(2-morpholin-4-ylethyl)- 1H-pyrazol-3-yl]-5- (trifluoromethyl)benzyl]-2-methyl- 2H-tetrazol-5-amine | 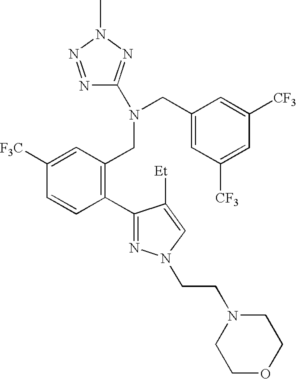 | 690.2 | 691.2 (M + 1) |
| 87 | N-[3,5-bis(trifluoromethyl)benzyl]-2- methyl-N-[2-(3-methyl-1H-pyrazol-1- yl)-5-(trifluoromethyl)benzyl]-2H- tetrazol-5-amine | 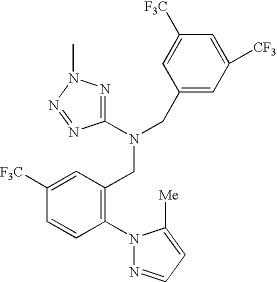 | 563.0 | 564.0 (M + 1) |
| 88 | N-[3,5-bis(trifluoromethyl)benzyl]-N- [2-(3,5-dimethyl-1H-pyrazol-1-yl)-5- (trifluoromethyl)benzyl]-2-methyl- 2H-tetrazol-5-amine | 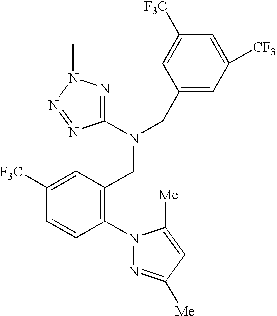 | 577.0 | 578.0 (M + 1) |
| 89 | Methyl [3,5- bis(trifluoromethyl)benzyl][2-(3,5- dimethyl-1H-pyrazol-1-yl)-5- (trifluoromethyl)benzyl]carbamate | 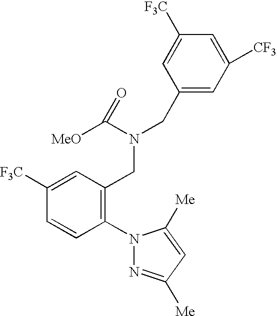 | 553.4 | 554.0 (M + 1) |
| 90 | N-[3,5-bis(trifluoromethyl)benzyl]-N- [2-imidazo[1,2-a]pyridin-2-yl-5- (trifluoromethyl)benzyl]-2-methyl- 2H-tetrazol-5-amine | 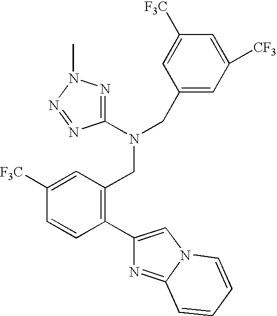 | 559.4 | 600.1 (M + 1) |
| 91 | N-[3,5-bis(trifluoromethyl)benzyl]-N- [2-(3-ethylimidazo[1,2-a]pyridin-2- yl)-5-(trifluoromethyl)benzyl]-2- methyl-2H-tetrazol-5-amine | 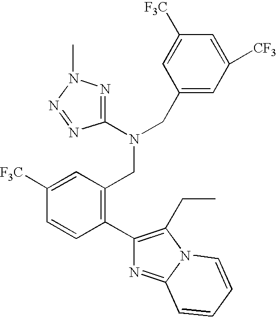 | 627.5 | 628.1 (M + 1) |
| 92 | N-[3,5-bis(trifluoromethyl)benzyl]-N- [2-(2-cyclopentyl-5-ethyl-1,3-thiazol- 4-yl)-5-(trifluoromethyl)benzyl]-2- methyl-2H-tetrazol-5-amine | 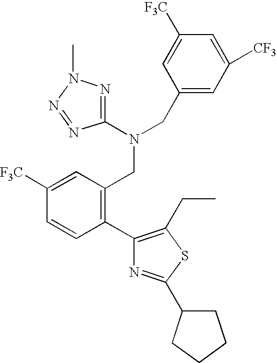 | 662.6 | 663.3 (M + 1) |
| 93 | N-[3,5-bis(trifluoromethyl)benzyl]-N- [2-[5-ethyl-2-(methoxymethyl)-1,3- thiazol-4-yl]-5- (trifluoromethyl)benzyl]-2-methyl- 2H-tetrazol-5-amine | 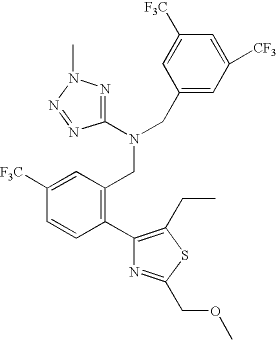 | 638.5 | 639.3 (M + 1) |
| 94 | N-[3,5-bis(trifluoromethyl)benzyl]-N- [2-[5-ethyl-2-(2,2,2-trifluoroethyl)- 1,3-thiazol-4-yl]-5- (trifluoromethyl)benzyl]-2-methyl- 2H-tetrazol-5-amine | 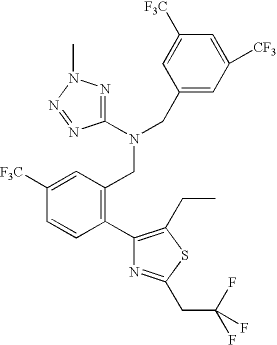 | 676.5 | 677.2 (M + 1) |
| 95 | N-[3,5-bis(trifluoromethyl)benzyl]-N- [2-[5-ethyl-2-(tetrahydro-2H-pyran- 4-yl)-1,3-thiazol-4-yl]-5- (trifluoromethyl)benzyl]-2-methyl- 2H-tetrazol-5-amine | 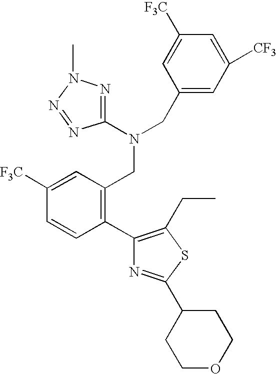 | 678.6 | 679.3 (M + 1) |
| 96 | N-[3,5-bis(trifluoromethyl)benzyl]-N- [2-[2-(cyclopropylmethyl)-5-ethyl- 1,3-thiazol-4-yl]-5- (trifluoromethyl)benzyl]-2-methyl- 2H-tetrazol-5-amine | 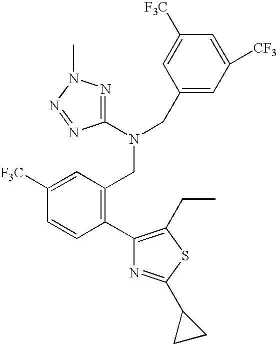 | 648.6 | 649.3 (M + 1) |
| 97 | 4-[2-{[[3,5- Bis(trifluoromethyl)benzyl](2-methyl- 2H-tetrazol-5-yl)amino]methyl}-4- (trifluoromethyl)phenyl]-5- ethylpyrimidine-2-carbonitrile | 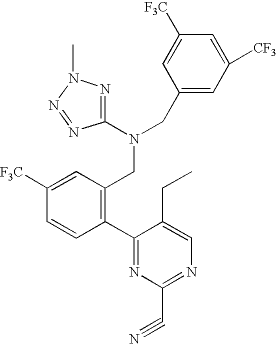 | 614.5 | 615.7 (M + 1) |
| 98 | 4-[2-{[[3,5- Bis(trifluoromethyl)benzyl](2-methyl- 2H-tetrazol-5-yl)amino]methyl}-4- (trifluoromethyl)phenyl]-5- ethylpyrimidine-2-carboxylic acid | 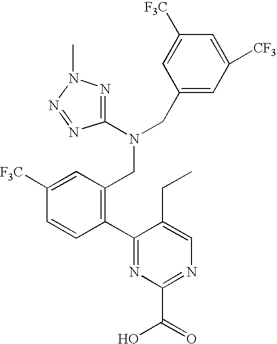 | 633.4 | 634.4 (M + 1) |
| 99 | Ethyl 4-[2-{[[3,5- bis(trifluoromethyl)benzyl](2-methyl- 2H-tetrazol-5-yl)amino]methyl}-4- (trifluoromethyl)phenyl]-5-ethyl-1,3- thiazole-2-carboxylate | 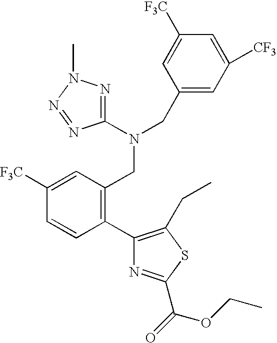 | 666.5 | 667.2 (M + 1) |
| 100 | Ethyl ({4-[2-{[[3,5- bis(trifluoromethyl)benzyl](2-methyl- 2H-tetrazol-5-yl)amino]methyl)-4- (trifluoromethyl)phenyl]-5- ethylpyrimidin-2-yl}oxy)acetate | 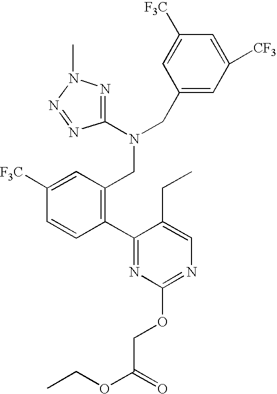 | 691.5 | 692.2 (M + 1) |
| 101 | Methyl 3-{4-[2-{[[3,5- bis(trifluoromethyl)benzyl](2-methyl- 2H-tetrazol-5-yl)amino]methyl}-4- (trifluoromethyl)phenyl]-5-ethyl-1,3- thiazol-2-yl}propanoate | 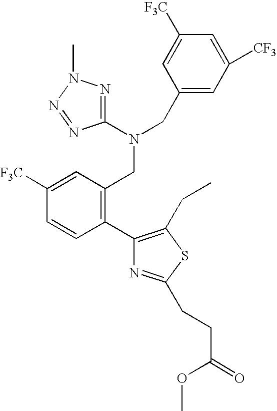 | 680.5 | 681.2 (M + 1) |
| 102 | 4-[2-{[[3,5- Bis(trifluoromethyl)benzyl](2-methyl- 2H-tetrazol-5-yl)amino]methyl}-4- (trifluoromethyl)phenyl]-5-ethyl-1,3- thiazole-2-carboxylic acid | 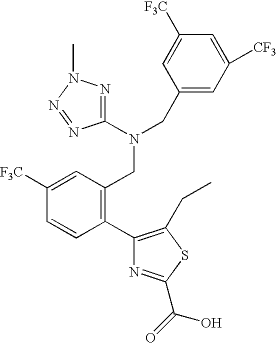 | 638.5 | 639.1 (M + 1) |
| 103 | 3-{4-[2-{[[3,5- Bis(trifluoromethyl)benzyl](2-methyl- 2H-tetrazol-5-yl)amino]methyl}-4- (trifluoromethyl)phenyl]-5-ethyl-1,3- thiazol-2-yl}propanoic acid | 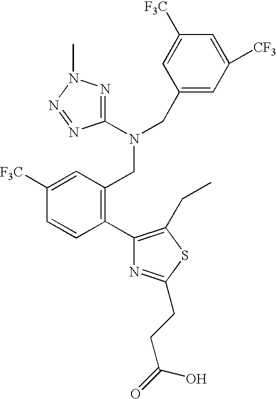 | 666.5 | 667.4 (M + 1) |
| 104 | {4-[2-{[[3,5- Bis(trifluoromethyl)benzyl](2-methyl- 2H-tetrazol-5-yl)amino]methyl}-4- (trifluoromethyl)phenyl]-5- ethylpyrimidin-2-yl}acetic acid | 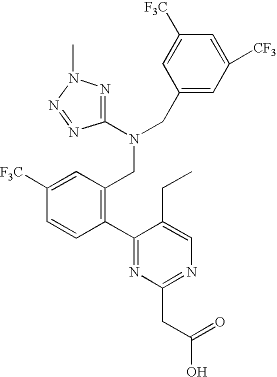 | 647.5 | 648.2 (M + 1) |
| 105 | N-[3,5-bis(trifluoromethyl)benzyl]-N- [2-[5-ethyl-2-(4-fluorophenyl)-1,3- thiazol-4-yl]-5- (trifluoromethyl)benzyl]-2-methyl- 2H-tetrazol-5-amine | 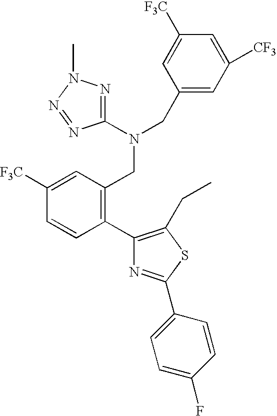 | 688.1 | 689.4 (M + 1) |
| 106 | N-[3,5-bis(trifluoromethyl)benzyl]-N- [2-{5-ethyl-2-[2-(1H-pyrrol-1- yl)ethyl]-1,3-thiazol-4-yl}-5- (trifluoromethyl)benzyl]-2-methyl- 2H-tetrazol-5-amine | 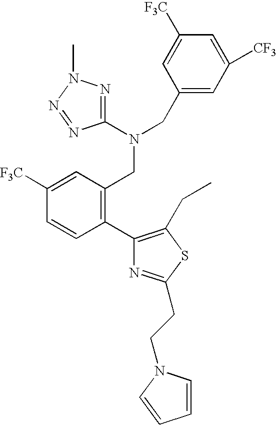 | 687.1 | 688.5 (M + 1) |
| 107 | Ethyl 5-{4-[2-{[[3,5- bis(trifluoromethyl)benzyl](2-methyl- 2H-tetrazol-5-yl)amino]methyl}-4- (trifluoromethyl)phenyl]-5-ethyl-1,3- thiazol-2-yl}pentanoate | 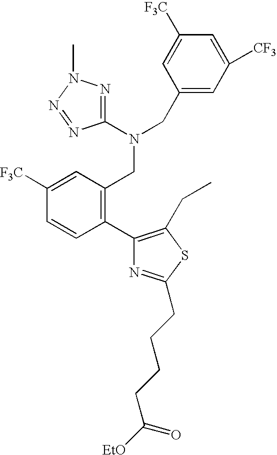 | 722.2 | 723.4 (M + 1) |
| 108 | N-[3,5-bis(trifluoromethyl)benzyl]-N- [2-[5-ethyl-2-(2,3,5,6- tetramethylphenyl)-1,3-thiazol-4-yl]- 5-(trifluoromethyl)benzyl]-2-methyl- 2H-tetrazol-5-amine | 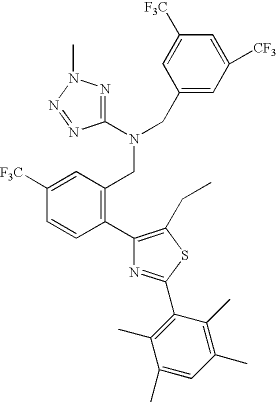 | 726.2 | 727.5 (M + 1) |
| 109 | N-({4-[2-{[[3,5- bis(trifluoromethyl)benzyl](2-methyl- 2H-tetrazol-5-yl)amino]methyl}-4- (trifluoromethyl)phenyl]-5-ethyl-1,3- thiazol-2-yl}methyl)acetamide | 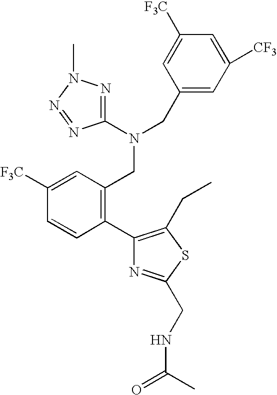 | 665.1 | 666.4 (M + 1) |
| 110 | N-[3,5-bis(trifluoromethyl)benzyl]-N- [2-(5-ethyl-2-pyridin-3-yl-1,3-thiazol- 4-yl)-5-(trifluoromethyl)benzyl]-2- methyl-2H-tetrazol-5-amine | 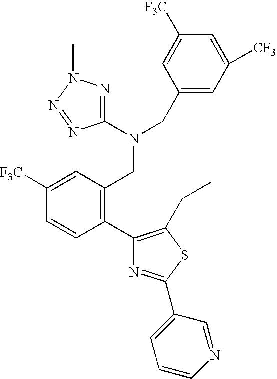 | 671.1 | 672.4 (M + 1) |
| 111 | N-({4-[2-{[[3,5- bis(trifluoromethyl)benzyl](2-methyl- 2H-tetrazol-5-yl)amino]methyl}-4- (trifluoromethyl)phenyl]-5-ethyl-1,3- thiazol-2- yl}methyl)methanesulfonamide | 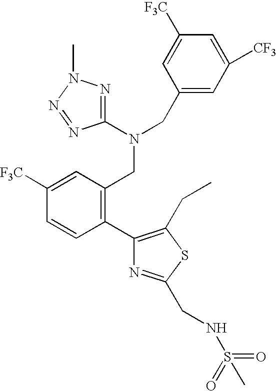 | 701.1 | 702.3 (M + 1) |
| 112 | N-[3,5-bis(trifluoromethyl)benzyl]-N- [2-[2-(3,5-dimethylpyridin-2-yl)-5- ethyl-1,3-thiazol-4-yl]-5- (trifluoromethyl)benzyl]-2-methyl- 2H-tetrazol-5-amine | 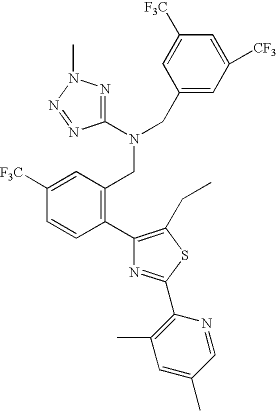 | 699.1 | 700.4 (M + 1) |
| 113 | N-[3,5-bis(trifluoromethyl)benzyl]-N- [2-{5-ethyl-2-[2-(1H-pyrazol-1- yl)methyl]-1,3-thiazol-4-yl}-5- (trifluoromethyl)benzyl]-2-methyl- 2H-tetrazol-5-amine | 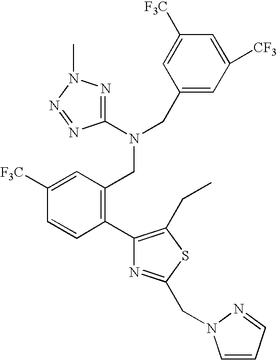 | 688.1 | 689.4 (M + 1) |
| 114 | 2-{4-[2-{[[3,5- Bis(trifluoromethyl)benzyl](2-methyl- 2H-tetrazol-5-yl)amino]methyl}-4- (trifluoromethyl)phenyl]-5-ethyl-1,3- thiazol-2-yl}isonicotinonitrile | 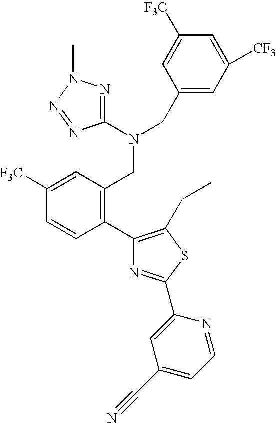 | 696.1 | 697.4 (M + 1) |
| 115 | N-[3,5-bis(trifluoromethyl)benzyl]-N- [2-[5-ethyl-2-(2-phenylethyl)-1,3- thiazol-4-yl]-5- (trifluoromethyl)benzyl]-2-methyl- 2H-tetrazol-5-amine | 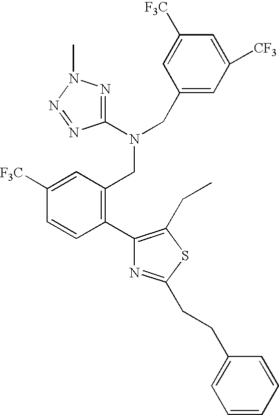 | 698.1 | 699.4 (M + 1) |
| 116 | N-({4-[2-{[[3,5- bis(trifluoromethyl)benzyl](2-methyl- 2H-tetrazol-5-yl)amino]methyl}-4- (trifluoromethyl)phenyl]-5-ethyl-1,3- thiazol-2-yl}methyl)benzamide | 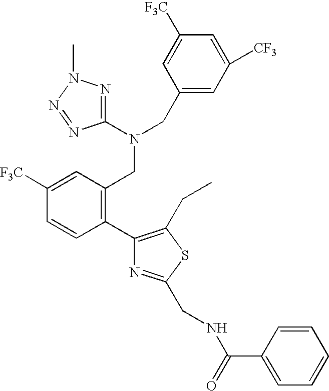 | 727.1 | 728.5 (M + 1) |
| 117 | N-[3,5-bis(trifluoromethyl)benzyl]-N- [2-(2-butyl-5-ethyl-1,3-thiazol-4-yl)- 5-(trifluoromethyl)benzyl]-2-methyl- 2H-tetrazol-5-amine | 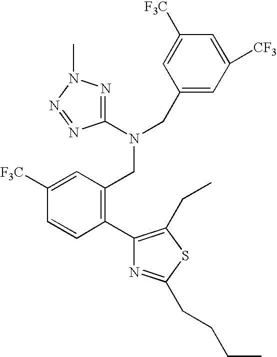 | 650.1 | 651.4 (M + 1) |
| 118 | N-[3,5-bis(trifluoromethyl)benzyl]-N- [2-[2-(2,4-difluorophenyl)-5-ethyl- 1,3-thiazol-4-yl]-5- (trifluoromethyl)benzyl]-2-methyl- 2H-tetrazol-5-amine | 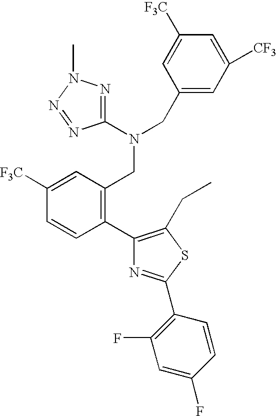 | 706.1 | 707.4 (M + 1) |
| 119 | Methyl 4-{4-[2-{[[3,5- bis(trifluoromethyl)benzyl](2-methyl- 2H-tetrazol-5-yl)amino]methyl}-4- (trifluoromethyl)phenyl]-5-ethyl-1,3- thiazol-2-yl}benzoate | 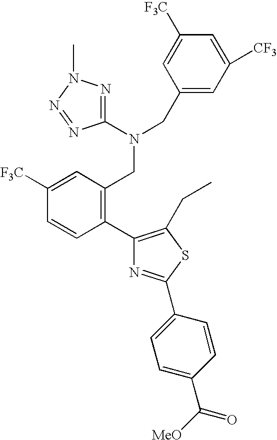 | 728.1 | 729.4 (M + 1) |
| 120 | N-[3,5-bis(trifluoromethyl)benzyl]-N- [2-(5-ethyl-2-phenyl-1,3-thiazol-4- yl)-5-(trifluoromethyl)benzyl]-2- methyl-2H-tetrazol-5-amine | 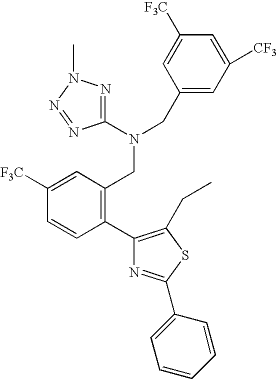 | 670.1 | 671.4 (M + 1) |
| 121 | N-[3,5-bis(trifluoromethyl)benzyl]-N- [2-{5-ethyl-2-[(5-methyl-1H- imidazol-4-yl)methyl]-1,3-thiazol-4- yl}-5-(trifluoromethyl)benzyl]-2- methyl-2H-tetrazol-5-amine | 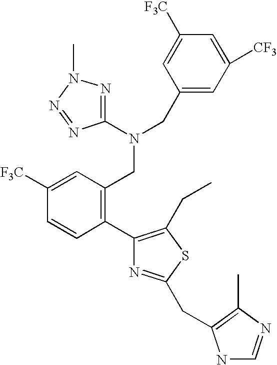 | 688.1 | 689.4 (M + 1) |
| 122 | N-[3,5-bis(trifluoromethyl)benzyl]-N- [2-[5-ethyl-2-(3-nitrophenyl)-1,3- thiazol-4-yl]-5- (trifluoromethyl)benzyl]-2-methyl- 2H-tetrazol-5-amine | 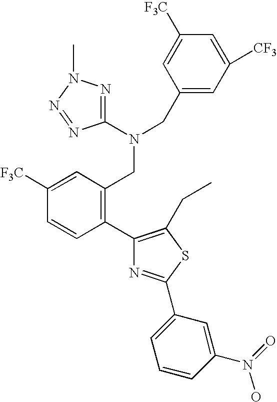 | 715.1 | 716.4 (M + 1) |
| 123 | N-[3,5-bis(trifluoromethyl)benzyl]-N- [2-[2-(2,2-dimethylpropyl)-5-ethyl- 1,3-thiazol-4-yl]-5- (trifluoromethyl)benzyl]-2-methyl- 2H-tetrazol-5-amine | 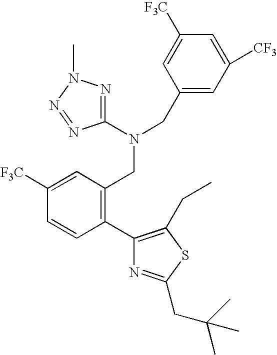 | 664.2 | 665.4 (M + 1) |
| 124 | N-[3,5-bis(trifluoromethyl)benzyl]-N- [2-[2-(2,2-dimethylpropyl)-5-ethyl- 1,3-thiazol-4-yl]-5- (trifluoromethyl)benzyl]-2-methyl- 2H-tetrazol-5-amine | 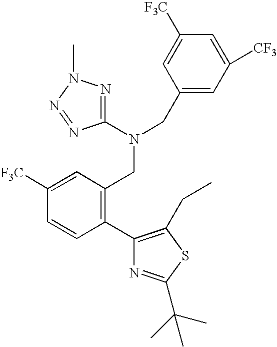 | 650.1 | 651.4 (M + 1) |
| 125 | N-[3,5-bis(trifluoromethyl)benzyl]-N- [2-(5-ethyl-2-isopropyl-1,3-thiazol-4- yl)-5-(trifluoromethyl)benzyl]-2- methyl-2H-tetrazol-5-amine | 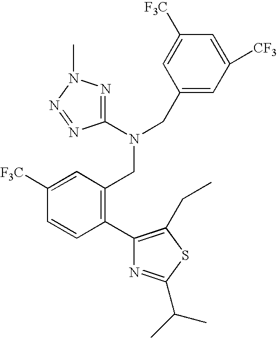 | 636.1 | 637.4 (M + 1) |
| 126 | N-[3,5-bis(trifluoromethyl)benzyl]-N- [2-(2-cyclopropyl-5-ethyl-1,3-thiazol- 4-yl)-5-(trifluoromethyl)benzyl]-2- methyl-2H-tetrazol-5-amine | 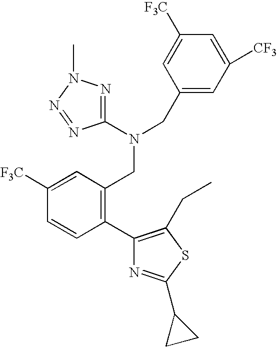 | 634.1 | 635.3 (M + 1) |
| 127 | N-[3,5-bis(trifluoromethyl)benzyl]-N- [2-(5-ethyl-2-propyl-1,3-thiazol-4-yl)- 5-(trifluoromethyl)benzyl]-2-methyl- 2H-tetrazol-5-amine | 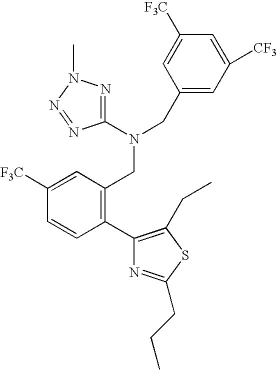 | 636.1 | 637.4 (M + 1) |
| 128 | N-[3,5-bis(trifluoromethyl)benzyl]-N- [2-[5-ethyl-2-(3-phenylpropyl)-1,3- thiazol-4-yl]-5- (trifluoromethyl)benzyl]-2-methyl- 2H-tetrazol-5-amine | 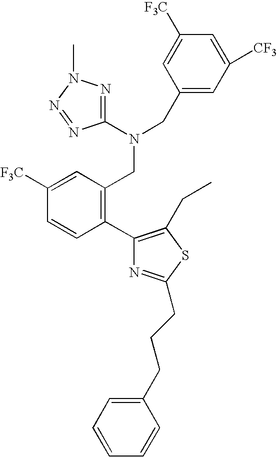 | 712.2 | 713.4 (M + 1) |
| 129 | N-[2-(2-benzyl-5-ethyl-1,3-thiazol-4- yl)-5-(trifluoromethyl)benzyl]-N-[3,5- bis(trifluoromethyl)benzyl]-2-methyl- 2H-tetrazol-5-amine | 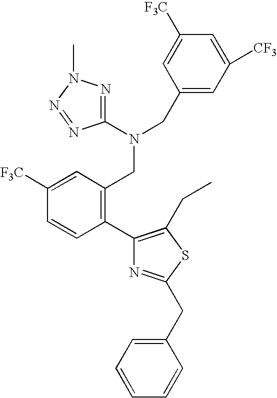 | 684.1 | 685.4 (M + 1) |
| 130 | N-[3,5-bis(trifluoromethyl)benzyl]-N- [2-[5-ethyl-2-(1-ethylpropyl)-1,3- thiazol-4-yl]-5- (trifluoromethyl)benzyl]-2-methyl- 2H-tetrazol-5-amine | 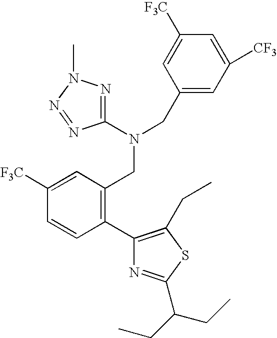 | 664.2 | 665.4 (M + 1) |
| 131 | N-[3,5-bis(trifluoromethyl)benzyl]-N- [2-[5-ethyl-2-(4-methylcyclohex-3- en-1-yl)-1,3-thiazol-4-yl]-5- (trifluoromethyl)benzyl]-2-methyl- 2H-tetrazol-5-amine | 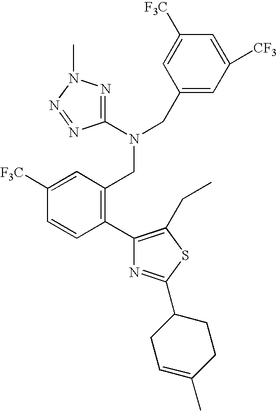 | 668.2 | 689.4 (M + 1) |
| 132 | N-[3,5-bis(trifluoromethyl)benzyl]-N- [2-(2,5-diethyl-1,3-thiazol-4-yl)-5- (trifluoromethyl)benzyl]-2-methyl- 2H-tetrazol-5-amine | 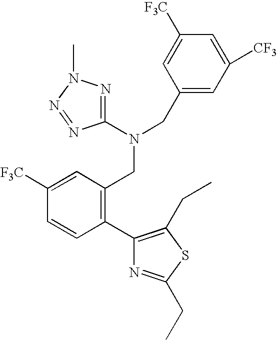 | 622.1 | 623.3 (M + 1) |
| 133 | N-[3,5-bis(trifluoromethyl)benzyl]-N- [2-(5-ethyl-2-isobutyl-1,3-thiazol-4- yl)-5-(trifluoromethyl)benzyl]-2- methyl-2H-tetrazol-5-amine | 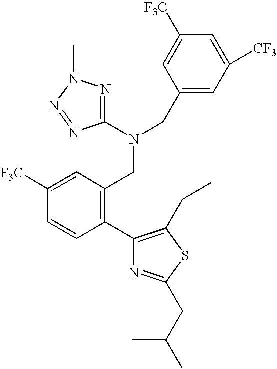 | 650.1 | 651.4 (M + 1) |
| 134 | Methyl 4-[2-{[[3,5- bis(trifluoromethyl)benzyl](2-methyl- 2H-tetrazol-5-yl)amino]methyl}-4- (trifluoromethyl)phenyl]-5-ethyl-1,3- thiazole-2-carboxylate | 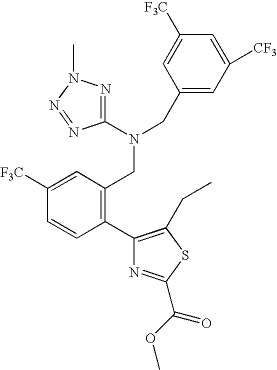 | 652.1 | 653.3 (M + 1) |
| 135 | N-[3,5-bis(trifluoromethyl)benzyl]-N- [2-[2-(1,1-dimethylpropyl)-5-ethyl- 1,3-thiazol-4-yl]-5- (trifluoromethyl)benzyl]-2-methyl- 2H-tetrazol-5-amine | 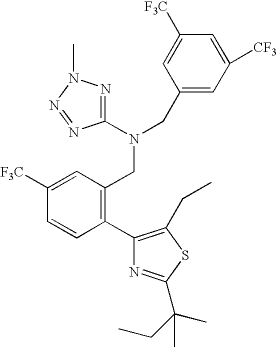 | 664.2 | 665.4 (M + 1) |
| 136 | N-[3,5-bis(trifluoromethyl)benzyl]-N- [2-(2-cyclohexyl-5-ethyl-1,3-thiazol- 4-yl)-5-(trifluoromethyl)benzyl]-2- methyl-2H-tetrazol-5-amine | 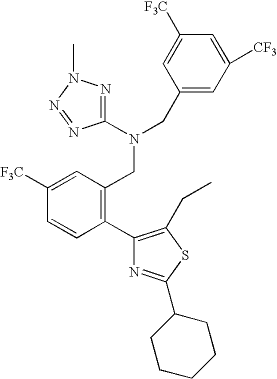 | 676.2 | 677.4 (M + 1) |
| 137 | 7-[2-{[[3,5- Bis(trifluoromethyl)benzyl](2-methyl- 2H-tetrazol-5-yl)amino]methyl}-4- (trifluoromethyl)phenyl]-6- ethylpyrazolo[1,5-a]pyrimidine-3- carbonitrile | 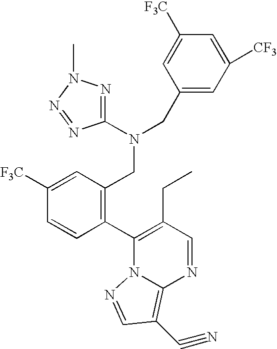 | 653.5 | 654.4 (M + 1) |
| 138 | N-[3,5-bis(trifluoromethyl)benzyl]-N- [2-[5-ethyl-2-(methoxymethyl)-1H- imidazol-4-yl]-5- (trifluoromethyl)benzyl]-2-methyl- 2H-tetrazol-5-amine | 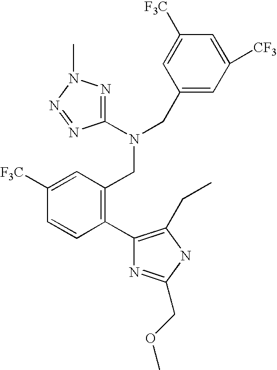 | 621.5 | 623.4 (M + 1) |
| 139 | Methyl 7-[2-{[[3,5- bis(trifluoromethyl)benzyl](2-methyl- 2H-tetrazol-5-yl)amino]methyl}-4- (trifluoromethyl)phenyl]-6- ethyl[1,2,4]triazolo[1,5-a]pyrimidine- 2-carboxylate | 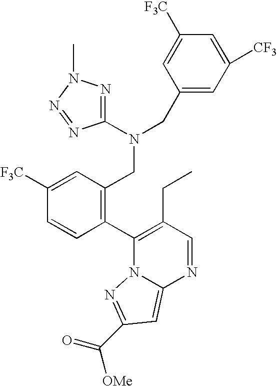 | 687.5 | 688.4 (M + 1) |
| 140 | N-[3,5-bis(trifluoromethyl)benzyl]-N- [2-(3-ethyl-7-methylimidazo[1,2- a]pyridin-2-yl)-5- (trifluoromethyl)benzyl]-2-methyl- 2H-tetrazol-5-amine | 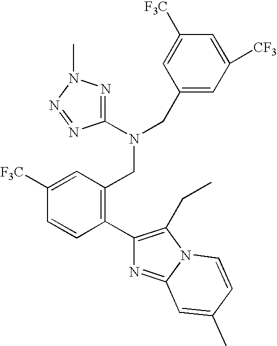 | 641.5 | 642.5 (M + 1) |
| 141 | 7-[2-{[[3,5- Bis(trifluoromethyl)benzyl](2-methyl- 2H-tetrazol-5-yl)amino]methyl}-4- (trifluoromethyl)phenyl]-6- ethylpyrazolo[1,5-a]pyrimidine-3- carboxamide | 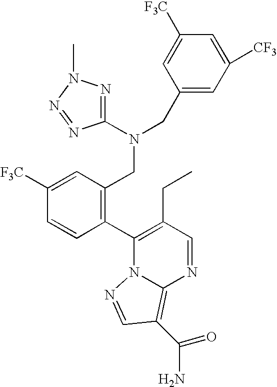 | 671.5 | 672.3 (M + 1) |
| 142 | N-[3,5-bis(trifluoromethyl)benzyl]-N- [2-(5-ethyl-2-methyl-1H-imidazol-4- yl)-5-(trifluoromethyl)benzyl]-2- methyl-2H-tetrazol-5-amine | 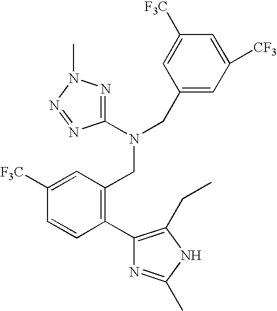 | 591.4 | 592.4 (M + 1) |
| 143 | ({4-[2-{[[3,5- Bis(trifluoromethyl)benzyl](2-methyl- 2H-tetrazol-5-yl)amino]methyl}-4- (trifluoromethyl)phenyl]-5- ethylpyrimidin-2-yl}oxy)acetic acid | 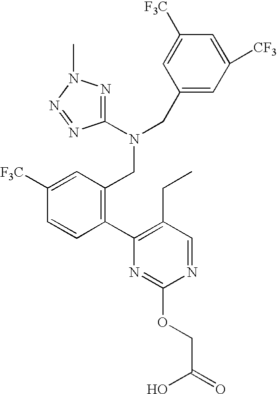 | 663 | 664.2 (M + 1) |

### Preparation 24

### 3-(3-Bromo-4-methoxy-phenyl)-acrylic acid methyl ester

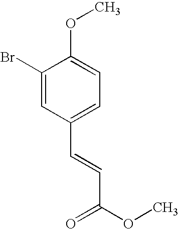

To a solution of (2E)-3-(3-bromo-4-methoxyphenyl)acrylic acid (1.6 g, 6.22 mmol) in 20% methanol in toluene (30 mL) was added 2.0M trimethylsilyidiazomethane in ethyl ether (6 mL, 12.4 mmol). The resulting yellow solution was quenched with glacial acetic acid until the reaction turned colorless. The solvent was evaporated to give the title compound as a tan solid (1.75 g).

¹H NMR (300 MHz, CHLOROFORM-D) δ ppm 7.73 (d, J=2.3 Hz, 1H) 7.57 (d, J=16 Hz, 1H) 7.43 (dd, J=8.5, 2.3 Hz, 1H) 6.89 (d, J=8.5 Hz, 1H) 6.31 (d, J=16 Hz, 1H) 3.92 (s, 3H) 3.79 (s, 3H) MS: Calc: 271.11. Found: (GC) 270; (M, ⁷⁹Br isotope).

### Preparation 25

### 3-[4-Methoxy-3-(4,4,5,5-tetramethyl-[1,3,2]dioxaborolan-2-yl)phenyl]-acrylic acid methyl ester

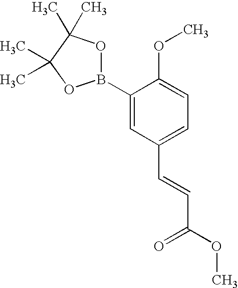

To a flask charged with 3-(3-bromo-4-methoxy-phenyl)-acrylic acid methyl ester (869 mg, 3.2 mmol) was added 1,4-dioxane (16 mL) followed by 4,4,5,5-tetramethyl-2-(4,4,5,5-tetramethyl-1,3,2-dioxaborolan-2-yl)-1,3,2-dioxaborolane (1.22 g, 4.8 mmol), potassium acetate (KOAc) (471 mg, 4.8 mmol) and tricyclohexyl phosphine (PCy₃) (180 mg, 0.64 mmol). The air was purged with nitrogen (N₂) and tris(dibenzylideneacetone)dipalladium (147 mg, 0.16 mmol) was added. The mixture was heated at 80° C. overnight. The reaction mixture was cooled to room temperature, diluted with diethyl ether and filtered over a pad of celite and silica gel. The mother liquor was washed with water three times. The organic layer was dried over anhydrous sodium sulfate, filtered and concentrated. The residue was purified by flash chromatography (silica gel) (eluting with 15-35% ethyl acetate in hexanes) to afford the title compound as a brownish solid (620 mg).

¹H NMR (300 MHz, CHLOROFORM-D) δ ppm 7.86 (d, J=2.5 Hz, 1H) 7.65 (d, J=16 Hz, 1H) 7.55 (dd, J=8.6, 2.4 Hz, 1H) 6.85 (d, J=8.7 Hz, 1H), 6.34 (d, J=16 Hz, 1H) 3.85 (s, 3H) 3.77 (s, 3H) 1.35 (s, 12H). MS: Calc: 318.17. Found: (GC) 318; (M).

### Example 144

### 3-(2′-{[(3,5-Bis-trifluoromethyl-benzyl)-(2-methyl-2H-tetrazol-5-yl)-amino]-methyl}-6-methoxy-4′-trifluoromethyl-biphenyl-3-yl)-acrylic acid methyl ester

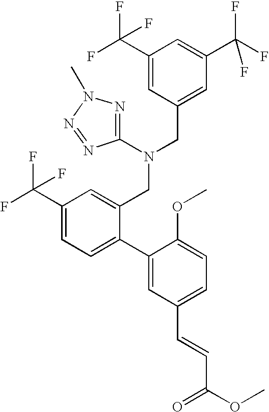

To a solution of N-(2-bromo-5-(trifluoromethyl)benzyl)-N-(3,5-bis(trifluoromethyl)benzyl)-2-methyl-2H-tetrazol-5-amine (147 mg, 0.261 mmol) in dimethylformamide (1 mL) was added 3-[4-methoxy-3-(4,4,5,5-tetramethyl-[1,3,2]dioxaborolan-2-yl)-phenyl]-acrylic acid methyl ester (100 mg, 0.314 mmol) followed by potassium phosphate (205 mg; 0.965 mmol) and tetrakis(triphenylphosphine)palladium (15 mg, 0.013 mmol). The resulting mixture was stirred at 110° C. for 16 hours. The reaction mixture was cooled to room temperature and diluted with 1:1 diethyl ether/ethyl acetate. The reaction mixture was filtered over a pad of celite/silica gel and rinsed with diethyl ether. The mother liquor was partitioned between water and ethyl ether three times. The organic layer was dried anhydrous sodium sulfate and concentrated. The residue was purified by flash chromatography (silica gel) (eluting with 20-40% ethyl acetate/hexanes) to afford the title compound (145 mg).

¹H NMR (300 MHz, CHLOROFORM-D) δ ppm 7.70 (s, 1H) 7.60 (d, J=16 Hz, 1H) 7.58 (dd, J=8.1, 1.3 Hz, 1H) 7.50 (m, 1H) 7.49 (m, 1H) 7.48 (s, 2H) 7.31 (d, J=7.9 Hz, 1H) 7.23 (d, J=2.3 Hz, 1H), 6.89 (d, J=8.5, 1H) 6.29 (d, J=16 Hz, 1H) 4.69 (d, J=16 Hz, 1H) 4.56 (d, J=16 Hz, 1H) 4.50 (d, J=16.0 Hz, 1H) 4.45 (d, J=16 Hz, 1H) 4.12 (s, 3H) 3.80 (s, 3H) 3.75 (s, 3H). MS (ES⁺) Calc: 673.53. Found: 674.0 (M+1).

### Example 145

### 3-(2′-{[(3,5-Bis-trifluoromethyl-benzyl)-(2-methyl-2H-tetrazol-5-yl)-amino]-methyl}-6-methoxy-4′-trifluoromethyl-biphenyl-3-yl)-acrylic acid

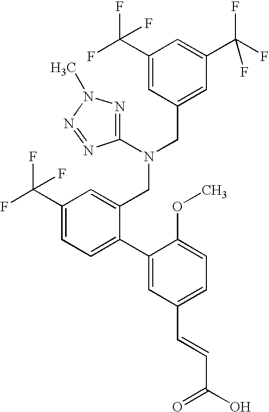

To a solution of 3-(2′-{[(3,5-bis-trifluoromethyl-benzyl)-2-methyl-2H-tetrazol-5-yl)-amino]-methyl}-6-methoxy-4′-trifluoromethyl-biphenyl-3-yl)-acrylic acid methyl ester (145 mg, 0.215 mmol) in tetrahydrofuran (1.5 mL) was added a solution of 1N sodium hydroxide (0.43 mL). Methanol (0.5 mL) and 1N sodium hydroxide (0.5 mL) were added and the reaction mixture was stirred for 2 days. The solvent was removed under vacuum and the residue partitioned between ethyl ether/ethyl acetate and water. The organic layer was washed with 2N hydrochloric acid and water two times. The organic layer was dried over anhydrous sodium sulfate, filtered and concentrated. The residue was crystallized from ether/hexanes to give the title compound as a white solid (121 mg).

¹H NMR (300 MHz, CHLOROFORM-D) δ ppm 7.71 (s, 1H) 7.68 (d, J=16 Hz, 1H) 7.58 (m, 1H) 7.51 (m, 1H) 7.50 (s, 1H) 7.48 (s, 2H) 7.31 (d, J=7.9 Hz, 1H) 7.25 (m, 1H), 6.90 (d, J=8.7 Hz, 1H) 6.29 (d, J=16 Hz, 1H) 4.68 (d, J=15.6 Hz, 1H) 4.57 (d, J=15.4 Hz, 1H) 4.50 (d, J=15.6 Hz, 1H) 4.45 (d, J=15.4 Hz, 1H) 4.12 (s, 3H) 3.76 (s, 3H) MS (ES⁺) Calc: 659.5. Found: 660.1 (M+1).

### Example 146

### 3-(2′-{[(3,5-Bis-trifluoromethyl-benzyl)-(2-methyl-2H-tetrazol-5-yl)-amino]-methyl}-6-methoxy-4′-trifluoromethyl-biphenyl-3-yl)propionic acid

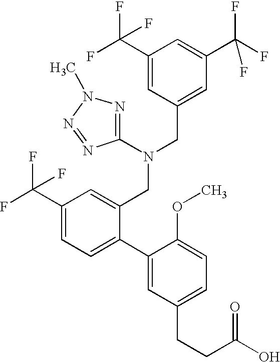

To a solution of 3-2′-[(3,5-bis-trifluoromethyl-benzyl)-2-methyl-2H-tetrazol-5-yl)-amino]-methyl)-6-methoxy-4′-trifluoromethyl-biphenyl-3-yl)acrylic acid (17 mg; 0.025 mmol) in ethanol (10 mL) was added 10% palladium on charcoal (8 mg). The resulting mixture was shaken on a Parr hydrogenator under 40 psi of hydrogen (H₂) for 4 hours. The reaction mixture was filtered over celite and rinsed with methanol. After concentration the residue was purified by flash chromatography (silica gel) (eluting with 40-70% ethyl acetate in hexanes) to afford the title compound (3.3 mg).

¹H NMR (300 MHz, CHLOROFORM-D) δ ppm 7.69 (s, 1H) 7.54 (d, J=7.9 Hz, 1H) 7.49 (s, 1H) 7.47 (s, 2H) 7.30 (d, J=7.9 Hz, 1H) 7.17 (dd, J=8.5, 2.3 Hz, 1H) 6.92 (d, J=2.3 Hz, 1H) 6.83 (d, J=8.5 Hz, 1H) 4.58 (s, 2H) 4.50 (d, J=16.2 Hz, 1H) 4.41 (d, J=16.2 Hz, 1H) 4.14 (s, 3H) 3.68 (s, 3H) 2.89 (m, 2H) 2.61 (m, 2H).

MS (ES⁻) Calc: 661.52. Found: 660.1 (M−1).

### Example 147

### 3-(2′-{[(3,5-Bis-trifluoromethyl-benzyl)-(2-methyl-2H-tetrazol-5-yl)amino]-methyl}6-methoxy-4′-trifluoromethyl-biphenyl-3-yl)-propionic acid methyl ester

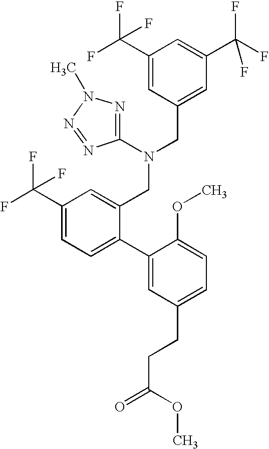

The title compound was prepared from 3-(2′-{[(3,5-bis-trifluoromethyl-benzyl)-2-methyl-2H-tetrazol-5-yl)-amino]-methyl}-6-methoxy-4′-trifluoromethyl-biphenyl-3-yl)-propionic acid using trimethylsilyidiazomethane using a method analogous to that described above for the synthesis of 3-(3-bromo-4-methoxy-phenyl)-acrylic acid methyl ester. The crude product was purified by flash chromatography (silica gel) (eluting with 10-20% ethyl acetate in hexanes) to give the title compound as a gum.

¹H NMR (300 MHz, CHLOROFORM-D) δ ppm 7.69 (s, 1H) 7.53 (d, J=7.9 Hz, 1H) 7.49 (s, 1H) 7.47 (s, 2H) 7.28 (d, J=7.9 Hz, 1H) 7.15 (dd, J=8.4, 2.3 Hz, 1H) 6.89 (d, J=2.3 Hz, 1H) 6.81 (d, J=8.4 Hz, 1H) 4.65 (d, J=16 Hz, 1H) 4.53 (d, J=16 Hz, 1H) 4.49 (d, J=16 Hz, 1H) 4.43 (d, J=16 Hz, 1H) 4.13 (s, 3H) 3.67 (s, 3H) 3.64 (s, 3H) 2.87 (m, 2H) 2.57 (m, 2H). MS (ES⁺) Calc: 675.55. Found: 676.4 (M+1).

### Example 148

### 3-(2′-{[(3,5-Bis-trifluoromethyl-benzyl)-(2-methyl-2H-tetrazol-5-yl)-amino]-methyl}-6-methoxy-4′-trifluoromethyl-biphenyl-3-yl)-propan-1-ol

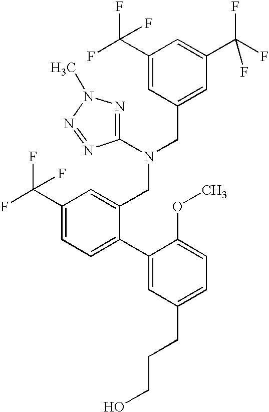

To a solution of 3-(2′-{[(3,5-bis-trifluoromethyl-benzyl)-(2-methyl-2H-tetrazol-5-yl)-amino]-methyl}-6-methoxy-4′-trifluoromethyl-biphenyl-3-yl)propionic acid (46 mg, 0.069 mmol) in tetrahydrofuran (2 mL) under nitrogen (N₂) was added borane•dimethyl•sulfide (13 □L, 0.139 mmol). The reaction mixture was stirred at room temperature overnight then quenched with 1N sodium hydroxide. The resulting mixture was stirred for 30 minutes then acidified with 2N hydrochloric acid. The mixture was extracted with ethyl acetate two times. The organic layers were dried over anhydrous sodium sulfate, filtered and concentrated. The residue was purified by flash chromatography (silica gel) (eluting with 30%-55% ethyl acetate in hexanes) to give the title compound (37 mg).

¹H NMR (300 MHz, CHLOROFORM-D) δ ppm 7.69 (s, 1H) 7.64 (d, J=8.0 Hz, 1H) 7.50 (s, 1H) 7.48 (s, 2H) 7.30 (d, J=8.0 Hz, 1H) 7.15 (dd, J=8.4, 2.3 Hz, 1H) 6.89 (d, J=2.3 Hz, 1H) 6.82 (d, J=8.4 Hz, 1H) 4.66 (d, J=15.8 Hz, 1H) 4.57 (d, J=15.8 Hz, 1H) 4.51 (d, J=16 Hz, 1H) 4.45 (d, J=16 Hz, 1H) 4.14 (s, 3H) 3.65 (t, J=6.4 Hz, 2H) 3.68 (s, 3H) 2.63 (m, 2H) 1.83 (m, 2H). MS (ES⁺) Calc: 647.54. Found: 648.4 (M+1).

### Preparation 26

### 3-[4-Methoxy-3-(4,4,5,5-tetramethyl-[1,3,2]dioxaborolan-2-yl)-phenyl]-but-2-enoic acid ethyl ester

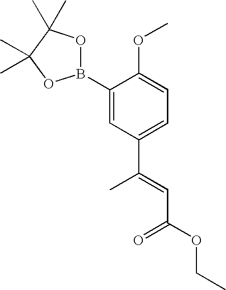

To a flask charged with ethyl (2E)-3-(3-bromo-4-methoxyphenyl)but-2-enoate (172 mg, 0.57 mmol) was added 1,4-dioxane (2.9 mL) followed by 4,4,5,5-tetramethyl-2-(4,4,5,5-tetramethyl-1,3,2-dioxaborolan-2-yl)-1,3,2-dioxaborolane (190 mg, 0.74 mmol), potassium acetate (KOAc) (85 mg, 0.861 mmol) and tricyclohexylphosphine (PCy₃) (27 mg, 0.098 mmol). The air was purged with nitrogen (N₂) and tris(dibenzylideneacetone)dipalladium (21 mg, 0.023 mmol) was added. The mixture was heated at 80° C. overnight. The reaction mixture was cooled to room temperature and diluted with diethyl ether. The reaction mixture was filtered over a pad of celite and silica gel and concentrated under vacuum. The residue was purified by flash chromatography (silica gel) (eluting with 10-30% ethyl acetate in hexanes) to afford the title compound as a yellow solid (128 mg).

¹H NMR (300 MHz, CHLOROFORM-D) δ ppm 7.81 (d, J=2.6 Hz, 1H) 7.53 (dd, J=8.7, 2.6 Hz, 1H) 6.84 (d, J=8.7 Hz, 1H), 6.12 (q, J=1.2 Hz, 1H) 4.2 (q, J=7.1 Hz, 2H) 3.85 (s, 3H) 2.56 (d, J=1.2 Hz, 3H) 1.35 (s, 12H) 1.31 (t, J=7.1 Hz, 3H).

MS (ES⁺) Calc: 346.23. Found: 347.0 (M+1).

### Example 149

### 3-(2′-{[(3,5-Bis-trifluoromethyl-benzyl)-(2-methyl-2H-tetrazol-5-yl)amino]-methyl}-6-methoxy-4′-trifluoromethyl-biphenyl-3-yl)-but-2-enoic acid ethyl ester

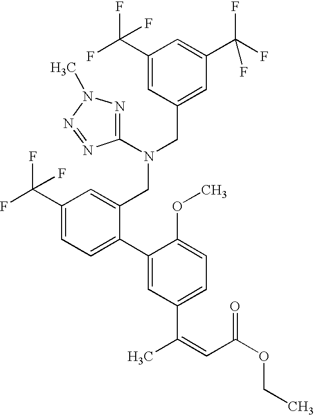

To a solution of N-(2-bromo-5-(trifluoromethyl)benzyl)-N-(3,5-bis(bifluoromethyl)benzyl)-2-methyl-2H-tetrazol-5-amine (412 mg, 0.732 mmol) in dimethylformamide (3.6 mL) was added 3-[4-methoxy-3-(4,4,5,5-tetramethyl-[1,3,2]dioxaborolan-2-yl)-phenyl]-but-2-enoic acid ethyl ester (381 mg, 1.1 mmol) followed by potassium phosphate (573 mg; 2.7 mmol) and tetrakis(triphenylphosphine)palladium (42 mg, 0.036 mmol). The resulting mixture was stirred at 80° C. for 16 hours. The reaction mixture was cooled to room temperature and diluted with diethyl ether. The reaction mixture was filtered over a pad of celite/silica gel and rinsed with diethyl ether. The mother liquor was partitioned between water and ethyl ether. The organic layer was dried anhydrous sodium sulfate and concentrated. The residue was purified by flash chromatography (silica gel) (eluting with 10-30% ethyl acetate/hexanes) to afford the title compound (529 mg).

¹H NMR (300 MHz, CHLOROFORM-D) δ ppm 7.70 (s, 1H) 7.57 (d, J=8.1 Hz, 1H) 7.49 (s, 2H) 7.47 (dd, J=8.9, 2.5 Hz, 1H) 7.32 (d, J=7.9 Hz, 1H) 7.22 (d, J=2.3 Hz, 1H), 6.88 (d, J=8.72, 1H) 6.07 (m, 1H) 4.67 (d, J=15.8 Hz, 1H) 4.65 (m, 2H) 4.47 (d, J=116.0 Hz, 1H) 4.19 (q, J=7.1 Hz, 2H) 4.12 (s, 3H) 3.74 (s, 3H) 2.53 (s, 3H), 1.30 (t, J=7.1 Hz, 3H). MS (ES⁺) Calc: 701.58. Found: 702.1 (M+1).

### Example 150

### 3-(2′-{[(3,5-Bis-trifluoromethyl-benzyl)-(2-methyl-2H-tetrazol-5-yl)-amino]-methyl}-6-methoxy-4′-trifluoromethyl-biphenyl-3-yl)-butyric acid ethyl ester

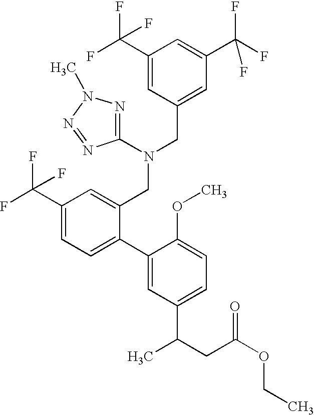

To a solution of 3-2′-{[(3,5-bis-trifluoromethyl-benzyl)-(2-methyl-2H-tetrazol-5-yl)-amino]-methyl}-6-methoxy-4′-trifluoromethyl-biphenyl-3-yl)but-2-enoic acid ethyl ester (60 mg; 0.085 mmol) in ethanol (10 mL) was added 10 wt. % palladium on activated carbon (20 mg). The resulting mixture was shaken on a Parr hydrogenation apparatus under 40 psi hydrogen (H₂). Reaction mixture was filtered over a pad of celite and rinsed with ethanol. The organic was concentrated to afford the title compound (59 mg). ¹H NMR (300 MHz, CHLOROFORM-D) δ ppm 7.69 (s, 1H) 7.54 (d, J=8.3 Hz, 1H) 7.50 (s, 1H) 7.49 (s, 2H) 7.30 (m, 1H) 7.18 (dd, J=8.5, 2.3 Hz, 1H) 6.94 (m, 1H) 6.84 (d, J=8.5 Hz, 1H) 4.64 (m, 1H) 4.58 (m, 1H) 4.48 (s, 2H) 4.14 (s, 3H) 4.05 (q, J=7.1 Hz, 2H) 3.68 (s, 3H) 3.22 (m, 1H) 2.51 (m, 2H) 1.24 (m, 3H), 1.17 (t, J=7.1 Hz, 3H). MS (ES⁺) Calc: 703.6. Found: 704.4 (M+1).

### Example 151

### 3-(2′-{[(3,5-Bis-trifluoromethyl-benzyl)-(2-methyl-2H-tetrazol-5-yl)amino]-methyl}-6-methoxy-4′-trifluoromethyl-biphenyl-3-yl)-butyric acid

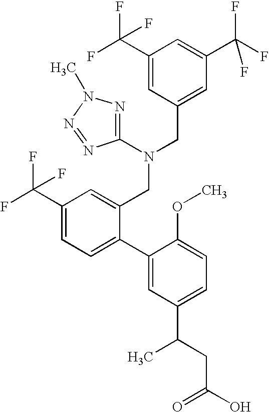

The title compound was prepared from 3-(2′-{[(3,5-bis-trifluoromethyl-benzyl)-(2-methyl-2H-tetrazol-5-yl)amino]-methyl}-6-methoxy-4′-trifluoromethyl-biphenyl-3-yl)-butyric acid ethyl ester using a hydrolysis procedure analogous to that described above for the synthesis of 3-(2′-{[(3,5-bis-trifluoromethyl-benzyl)-(2-methyl-2H-tetrazol-5-yl)-amino]-methyl}-6-methoxy-4′-trifluoromethyl-biphenyl-3-yl)-acrylic acid. The residue was purified by flash chromatography (silica gel) (eluting with 0-5% methanol in methylene chloride) to give the title compound as a gum.

¹H NMR (300 MHz, CHLOROFORM-D) δ ppm 7.70 (s, 1H) 7.55 (d, J=8.1 Hz, 1H) 7.49 (m, 1H) 7.48 (s, 2H) 7.31 (m, 1H) 7.19 (m, 1H) 6.95 (m, 1H) 6.85 (m, 1H) 4.60 (m, 2H) 4.45 (m, 2H) 4.13 (s, 3H) 3.69 (s, 3H) 3.20 (m, 1H) 2.54 (m, 2H) 1.27 (m, 3H). MS (ES⁺) Calc: 675.54. Found: 676.3 (M+1).

### Example 152

### 3-(2′-{[(3,5-Bis-trifluoromethyl-benzyl)-(2-methyl-2H-tetrazol-5-yl)-amino]-methyl}-6-methoxy-4′-trifluoromethyl-biphenyl-3-yl)-butyramide

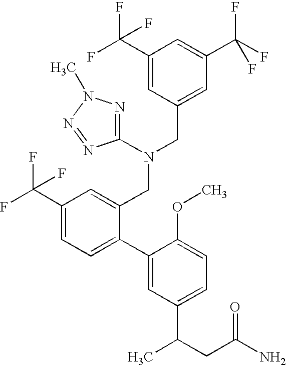

To a solution of 3-(2′-([(3,5-bis-trifluoromethyl-benzyl)-(2-methyl-2H-tetrazol-5-yl)amino]-methyl)-methoxy-4′-trifluoromethyl-biphenyl-3-yl)-butyric acid (27 mg, 0.040 mmol) in methylene chloride (2 mL) under nitrogen was added thionyl chloride (2 mL). After stirring for 1.5 hours at room temperature the solvent was evaporated and to the residue was added methylene chloride. The solvent was evaporated to dryness and this procedure was repeated twice more to complete the removal of thionyl chloride. To the residue was added a solution of 0.5M ammonia in 1,4-dioxane (6 mL). The reaction mixture was stirred overnight at room temperature. The reaction was concentrated and the residue was purified by flash chromatography (silica gel) (eluting with 0-2% methanol in methylene chloride) to give the title compound (29 mg).

¹H NMR (300 MHz, CHLOROFORM-D) δ ppm 7.71 (s, 1H) 7.55 (d, J=7.9 Hz, 1H) 7.50 (s, 2H) 7.47 (s, 1H) 7.31 (d, J=7.9 Hz, 1H) 7.20 (dd, J=8.5, 2.3 Hz, 1H) 6.96 (bs, 1H) 6.84 (d, J=8.5 Hz, 1H) 5.42 (bm, 2H) 4.59 (m, 2H) 4.49 (m, 2H) 4.13 (s, 3H) 3.69 (s, 3H) 3.25 (m, 1H) 2.42 (m, 2H) 1.28 (d, J=7.1 Hz, 3H).

MS (ES⁺) Calc: 674.56. Found: 675.2 (M+1).

### Preparation 27

### 3-Bromo-4-chloro-N-methylbenzamide

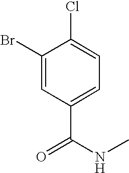

A solution of 3-bromo-4-chlorobenzoic acid (6.0 g, 25.48 mmol) in 250 mL methylene chloride was cooled to 0° C. and 0.5 mL dimethyl formamide was added. Oxalyl chloride (2.667 mL, 30.58 mmol) was then added dropwise to the solution. The mixture was stirred at 0° C. for 1 hour and then warmed to room temperature for 3 hours. Methyl amine (2M solution in THF, 50 mL) was added dropwise to the reaction mixture. The reaction mixture was stirred overnight. 250 mL methylene chloride was added to the reaction mixture. The solution was washed twice with 300 mL water and 300 mL saturated sodium bicarbonate solution. The organic layer was dried over magnesium sulfate and the solvent removed in vacuo to afford the title compound (6.15 g) as a solid. ¹H NMR (400 MHz, CDCL₃) δ ppm 7.84 (d, 1H, J=8 Hz), 7.71 (s, 1H), 7.43 (d, 1H, J=8 Hz), 7.4 (b, 1H), 2.88 (s, 3H).

### Preparation 28

### Tert-butyl(3-bromo-4-chlorobenzyl)methylcarbamate

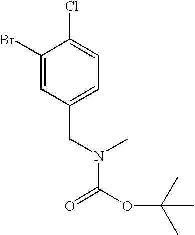

To a solution of 3-Bromo-4-chloro-N-methylbenzamide (6.1 g, 24.7 mmol) in 125 mL THF was added 50 mL of a 1.0M THF solution of Borane-THF complex. The resulting solution was heated to reflux over night. The reaction was cooled to room temperature, and quenched by the slow addition of 4N hydrochloric acid (20 ml). The resulting mixture was heated to reflux for 2 hours and cooled to room temperature. Sodium hydroxide solution (40 mL, 4N) was slowly added to the reaction mixture followed by the addition of 500 mL methylene chloride. The organic layer was collected, and washed twice with 500 mL water, 500 mL saturated brine, dried over magnesium sulfate and concentrated in vacuo to afford the secondary amine as a crude product (5.8 g). This crude product was dissolved in 150 mL methylene chloride. Di-tert-butyl dicarbonate (6.48 g, 29.7 mmol) was then added and the resulting solution was stirred at room temperature for 4 hours. The reaction mixture was washed three times with 150 mL water, dried over magnesium sulfate and the solvent removed in vacuo. The residue purified on silica chromatography (40 g Isco RediSep, gradient: 0% to 15% ethyl acetate in hexane) to give 21.72 g of the title compound as a colorless oil.

¹H NMR (400 MHz, CDCL₃) δ ppm 7.26 (s, 1H), 7.11 (m, 2H), 4.32 (m, 2H), 2.73 (s, 3H), 1.42 (s, 9H).

### Example 153

### 1-(2′-{[(3,5-Bis-trifluoromethyl-benzyl)-(2-methyl-2H-tetrazol-5-yl)-amino]-methyl}-6-methyl-4′-trifluoromethyl-biphenyl-3-yl)-ethanone

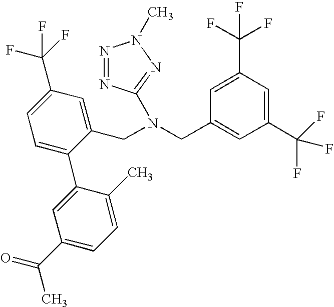

To a 10-20 ml Emrys™ Process Vial was added (3,5-bis-trifluoromethyl-benzyl)-(2-methyl-2H-tetrazol-5-yl)-[2-(4,4,5,5-tetramethyl-[1,3,2]dioxaborolan-2-yl)-5-trifluoromethyl-benzyl]-amine (2.2 g, 3.8 mmol), 1-(3-bromo-4-methyl-phenyl)-ethanone (0.988 g, 4.6 mmol), tetrakis(triphenylphosphine)palladium(0) powder (0.438 g, 0.38 mmol) followed by dioxane (14 ml) ethanol (7 ml) and aqueous sodium carbonate (2M, 7 ml), which were deoxygenated by bubbling nitrogen gas through stirred solvent for 20 minutes prior to use. The reaction vial was sealed then heated to 150° C. for 10 minutes under microwave irradiation (Emrys Optimizer™). The reaction was cooled to room temperature and partitioned between water (75 ml) and ethyl acetate (100 ml). The layers were separated and the aqueous phase was extracted with ethyl acetate (two times 50 ml). The combined organic layers were washed with saturated sodium chloride (NaCl) (100 ml), dried with sodium sulfate (Na₂SO₄), filtered and concentrated to a yellow residue, which was adsorbed onto silica gel and purified by flash chromatography (120 G, gradient from 5-20% ethyl acetate in hexanes, flow of 40 ml/min) to yield the title compound as a yellow oil (1.065 g, 44%). ¹H NMR (400 MHz, CDCL₃) δ ppm 7.83 (d, J=8 Hz, 1H) 7.72 (s, 1H) 7.64 (s, 1H) 7.61 (d, J=8 Hz, 1H) 7.55 (s, 1H) 7.51 (s, 2H) 7.33 (d, J=8 Hz, 1H) 7.28 (d, J=8 Hz, 1H) 4.56 (m, 2H) 4.44 (q, J=16 Hz, 2H) 4.10 (s, 3H) 2.53 (s, 3H) 2.09 (s, 3H); MS (ES+) Calc: 615.168. Found: 616.0 (M+1).

For examples 154-160, Analytical HPLC/MS was performed on a Waters 2795 system with Autosampler, UV detection (Waters DAD 996, Waters, Milford, Mass.) monitoring at 215 nm, ELSD detection (SEDEX 75, Sedere, Somerset, N.J.) and mass detection using a Micromass ZQ Spectrometer (Micromass, Manchester, UK). The mobile phase utilized was acetonitrile/water; containing 1% trifluoroacetic acid using a 5 minute gradient 25% to 95% (% acetonitrile) using an Atlantis dC18 4.6×50 mm, 5 um column (Waters, Milford, Mass.).

### Example 154

### (3,5-Bis-trifluoromethyl-benzyl)-[2′-methyl-5′-(1-morpholin-4-yl-ethyl)-4-trifluoromethyl-biphenyl-2-ylmethyl]-(2-methyl-2H-tetrazol-5-yl)amine

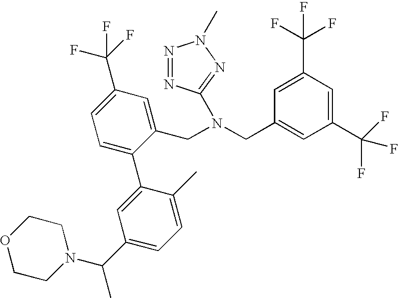

To a solution of 1-(2′-{[(3,5-Bis-trifluoromethyl-benzyl)-(2-methyl-2H-tetrazol-5-yl)-amino]-methyl}-6-methyl-4′-trifluoromethyl-biphenyl-3-yl)-ethanone (23 mg, 0.037 mmol) in titanium (IV) isopropoxide (0.5 ml) at room temperature was added morpholine (6.5 mg, 0.074 mmol) and stirred for 16 hours. Reaction was diluted with ethanol (2.0 ml) then sodium borohydride (2.8 mg, 0.074 mmol) was added in one portion and stirred for 2 hours. Reaction was diluted with dichloromethane (20 ml). To this was added aqueous ammonium hydroxide (10 ml), which formed a white precipitate. Solution was filtered through celite with dichloromethane rinse (2 times 5 ml). Organic was extracted with brine (20 ml) then dried over sodium sulfate, and concentrated to a white residue, which was dissolved in dimethyl sulfoxide (1 ml) and purified on prepHPLC [Shimadzu preparative HPLC xttera column 30×50 C18, 30-95%, 0.1% NaOH, 8 min gradient, 220UV] to yield 3.0 mg (12%) of the title compound as clear oil.

MS (ES⁺) Calc: 686.2. Found: 687.1 (M+1). LC-MS Retention time: 1.9 minutes.

### Example 155

### (3,5-Bis-trifluoromethyl-benzyl)-[5-(1-dimethylamino-ethyl)-2′-methyl-4-trifluoromethyl-biphenyl-2-ylmethyl]-(2-methyl-2H-tetrazol-5-yl)-amine

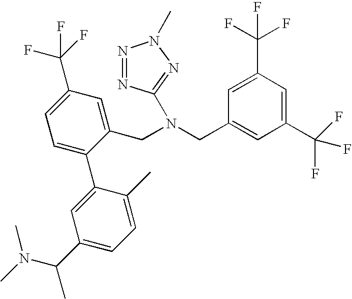

The title compound was prepared in the manner of Example 154 using the appropriate materials to yield 9.5 mg (30%) as a clear oil. MS (ES⁺) Calc: 644.2. Found: 645.1 (M+1). LC-MS Retention time: 2.5 minutes.

### Example 156

### Tert-butyl{[2′-({[3,5-bis(trifluoromethyl)benzyl](2-methyl-2H-tetrazol-5-yl)amino}methyl)-6-chloro-4′-(trifluoromethyl)biphenyl-3-yl]methyl}methylcarbamate

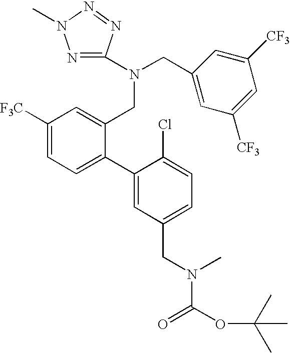

To a solution of N-(3,5-bis(trifluoromethyl)benzyl)-N-(5-(trifluoromethyl)-2-(4,4,5,5-tetramethyl-1,3,2-dioxaborolan-2-yl)benzyl)-2-methyl-2H-tetrazol-5-amine (305 mg, 0.5 mmol) in deoxygenated ethanol (5.0 mL) was added a solution of tert-butyl 3-bromo-4-chlorobenzylmethylcarbamate (167 mg, 0.5 mmol) in deoxygenated 1,4-dioxane (2.0 mL). Tetrakis(triphenylphosphine)palladium(0) (Pd(PPh₃)₄) (29 mg, 0.025 mmol) in deoxygenated 1,4-dioxane (2.0 mL) and 2 M aqueous sodium carbonate (Na₂CO₃) (1.5 mL, 3.0 mmol) were then added. The resulting mixture was stirred at 95° C. for 3 hours. The reaction mixture was concentrated, and partitioned between water and ethyl acetate. The organic layer was concentrated and the residue was purified by silica chromatography to afford the title compound (9.145 mg) as a white solid (Eluted with 5% to 30% ethyl acetate in hexane). ¹H NMR (400 MHz, CDCL₃) δ ppm 7.69 (s, 1H), 7.57 (d, 1H, J=8 Hz), 7.54 (s, 2H), 7.52 (s, 1H), 7.36 (d, 1H, J=8 Hz), 7.29 (d, 1H, J=8 Hz), 7.15 (d, 1H, J=7 Hz), 6.99 (d, 1H, J=8 Hz), 4.60 (d, 2H, J=15), 4.49 (m, 2H), 4.34 (s, 2H), 4.11 (s, 3H), 2.78 (t, 3H, J=15), 1.42 (s, 9H). MS (ES⁺) Calc: 736.2. Found: 737 (M+1).

### Example 157

### N-[3,5-bis(trifluoromethyl)benzyl]-N-({2′-chloro-5′-[(methylamino)methyl]-4-(trifluoromethyl)biphenyl-2-yl}methyl)-2-methyl-2H-tetrazol-5-amine

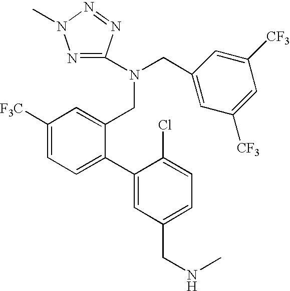

To a solution of tert-butyl {[2′-({[3,5-bis(trifluoromethyl)benzyl](2-methyl-2H-tetrazol-5-yl)amino}methyl)-6-chloro-4′-(trifluoromethyl)biphenyl-3-yl]methyl}methylcarbamate (134 mg, 0.18 mmol) in 2 mL of methylene chloride was added 2 ml trifluoroacetic acid. The resulting solution was stirred at room temperature under nitrogen for 4 hours. The solvent and excess trifluoroacetic acid were then removed under reduced pressure. The residue was dissolved in 10 mL methylene chloride, and washed with 20 mL 1N sodium hydroxide solution. The organic phase was dried over sodium sulfate and concentrated in vacuo to afford 108 mg title product as colorless oil. ¹H NMR (400 MHz, CDCL₃) δ ppm 7.70 (s, 1H), 7.58 (d, 1H, J=8 Hz), 7.52 (m, 3H), 7.35 (d, 1H, J=8 Hz), 7.29 (d, 1H, J=8 Hz), 7.24 (m, 1H), 7.10 (d, 1H, J=8 Hz), 4.54 (m, 4H), 4.12 (s, 3H), 3.70 (s, 2H), 2.43 (s, 3H). MS (ES⁺) Calc: 636.2. Found: 637 (M+1).

### Example 158

### N-[3,5-bis(trifluoromethyl)benzyl]-N-({2′-chloro-5′-[(dimethylamino)methyl]-4-(trifluoromethyl)biphenyl-2-yl}methyl)-2-methyl-2H-tetrazol-5-amine

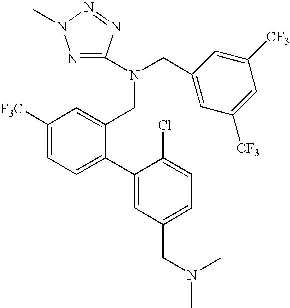

To a solution of N-[3,5-bis(trifluoromethyl)benzyl]-N-({2′-chloro-5′-[(methylamino)methyl]-4-(trifluoromethyl)biphenyl-2-yl}methyl)-2-methyl-2H-tetrazol-5-amine (10 mg, 0.016 mmol) in 2 mL of chloroform was added formaldehyde (37% aqueous solution, 4.4 uL, 0.16 mmol) and sodium triacetoxyboronhydride as a solid (22.2 mg, 0.1 mmol). The resulting mixture was stirred at room temperature overnight. 10 mL saturated sodium bicarbonate solution was added to the reaction mixture followed by 10 mL chloroform. The organic layer was collected, washed with saturated brine, and dried over sodium sulfate. The solvent was removed under reduced pressure. The residue purified on preparative TLC (developed with 50% ethyl acetate in hexane) to afford 8.4 mg title compound as colorless oil. MS (ES⁺) Calc: 650.2. Found: 651.3 (M+1). LC-MS retention time: 1.1 minutes.

### Example 159

### Methyl{[2′-({[3,5-bis(trifluoromethyl)benzyl](2-methyl-2H-tetrazol-5-yl)amino}methyl)-6-chloro-4′-(trifluoromethyl)biphenyl-3-yl]methyl}methylcarbamate

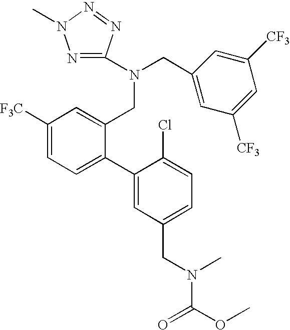

To a solution of N-[3,5-bis(trifluoromethyl)benzyl]-N-{2′-chloro-5′-[(methylamino)methyl]-4-(trifluoromethyl)biphenyl-2-yl}methyl)-2-methyl-2H-tetrazol-5-amine (10 mg, 0.016 mmol) in 4 mL methylene chloride was added triethyl amine (22 uL, 0.16 mmol) and methyl chloroformate (6.2 uL, 0.08 mmol). The resulting solution was stirred at room temperature overnight. The reaction mixture was concentrated under reduced pressure. 10 mL saturated sodium bicarbonate solution was added to the residue followed by 10 mL methylene chloride. The organic layer was washed with 10 mL water and 10 mL brine, dried over sodium sulfate, and concentrated in vacuo. The residue was purified on preparative TLC (developed with 50% ethyl acetate in hexane) to afford 8.0 mg the title compound as a colorless oil. MS (ES+) Calc: 694.2. Found: 695.3 (M+1). LC-MS retention time: 1.5 minutes.

### Example 160

### N-{[2′-({[3,5-bis(trifluoromethyl)benzyl](2-methyl-2H-tetrazol-5-yl)amino}methyl)-6-chloro-4′-(trifluoromethyl)biphenyl-3-yl]methyl}-N-methylmethanesulfonamide

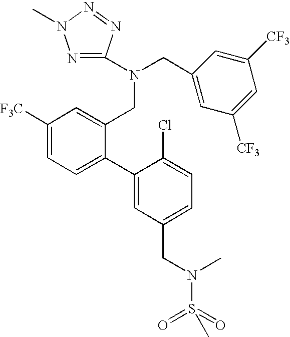

To a solution of N-[3,5-bis(trifluoromethyl)benzyl]-N-({2′-chloro-5′-[(methylamino)methyl]-4-(trifluoromethyl)biphenyl-2-yl}methyl)-2-methyl-2H-tetrazol-5-amine (10 mg, 0.016 mmol) in 4 mL methylene chloride was added triethylamine (22 uL, 0.16 mmol) and methane sulfonyl chloride (6.2 uL, 0.08 mmol). The resulting solution was stirred at room temperature overnight. The reaction mixture was concentrated under reduced pressure. 10 mL saturated sodium bicarbonate solution was added to the residue followed by 10 mL methylene chloride. The organic layer was washed with 10 mL water and 10 mL brine, dried over sodium sulfate, solvent removed. The residue was purified by preparative TLC (developed with 50% ethyl acetate in hexane) to afford 10.0 mg title compound as a colorless oil. MS (ES+) Calc: 714.3. Found: 715.3 (M+1). LC-MS retention time: 1.5 minutes.

Throughout this application, various publications are referenced. The disclosures of these publications in their entireties are hereby incorporated by reference into this application for all purposes. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound of Formula I 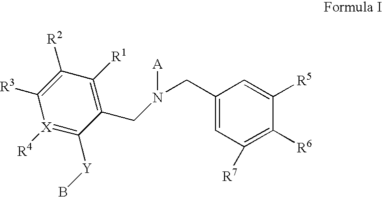
or a pharmaceutically acceptable salt of said compound; wherein
A is —COO(C₁-C₄)alkyl, cyano, —CHO, —CONH₂, —CO(C₁-C₄)alkyl, triazolyl, tetrazolyl, oxadiazolyl, isoxazolyl, pyrazolyl, or thiadiazolyl and A is optionally mono-, di- or tri-substituted with R⁰;
X is C or N, wherein if X is N, R⁴ is absent;
Y is a bond, —O—, —CR¹¹R¹²—, —CR¹¹R¹²—O—, or —O—CR¹¹R¹²—, wherein R¹¹ and R¹² are each independently hydrogen or (C₁-C₆)alkyl wherein said (C₁-C₆)alkyl is optionally substituted with one to nine halo, or R¹¹ and R¹² may be taken together to form a (C₃-C₆)cycloalkyl optionally substituted with one to nine halo;
B is aryl or heteroaryl wherein B is optionally mono-, di- or tri-substituted independently with —(C₀-C₆)alkyl-NR⁸R⁹, —(C₀-C₆)alkyl-CO—NR⁸R⁹, —(C₀-C₆)alkyl-CO—OR¹⁰, —(C₀-C₆)alkyl-NR¹³—(C₀-C₆)alkyl-CO—O—R¹⁰, —C₀-C₆)alkyl-NR¹³—(C₀-C₆)alkyl-CO—R¹⁴, —(C₀-C₆)alkyl-NR¹³—(C₀-C₆)alkyl-SO₂—R¹⁰, —(C₁-C₆)alkyl-O—CO—NR⁸R⁹, —O—(C₁-C₆)alkyl-CO—O—R¹⁰, —(C₂-C₆)alkenyl-CO—O—R¹⁰, —(C₀-C₆)alkyl-aryl, —(C₀-C₆)alkyl-heteroaryl, —O—(C₀-C₆)alkyl-aryl, —O—(C₀-C₆)alkyl-heteroaryl, —(C₀-C₆)alkyl-O-aryl, —(C₀-C₆)alkyl-O-heteroaryl, —(C₀-C₆)alkyl-heterocycle, —O—(C₀-C₆)alkyl-heterocycle, —(C₀-C₆)alkyl-(C₃-C₆)cycloalkyl, —O—(C₀-C₆)alkyl-(C₃-C₆)cycloalkyl, —(C₀-C₆)alkyl-(C₃-C₆)cycloalkenyl, halo, (C₂-C₆)alkynyl, (C₂-C₆)alkenyl, (C₁-C₆)alkyl, hydroxy, (C₁-C₆)alkoxy, (C₁-C₄)alkylthio, nitro, cyano, oxo, —CO—(C₁-C₆)alkyl, or —CO—O—(C₁-C₆)alkyl wherein said aryl, heteroaryl, heterocycle, cycloalkenyl, cycloalkyl, alkynyl, alkenyl, alkyl and alkoxy substituents are each optionally substituted independently with one to nine halo, one or two hydroxy, one or two (C₁-C₆)alkoxy, one or two amino, one or two nitro, cyano, oxo, or carboxy, wherein R⁸ and R⁹ are each independently hydrogen, (C₁-C₆)alkyl, or (C₁-C₆)alkoxy, wherein said alkyl is optionally substituted with one to nine halo; R¹⁰ is hydrogen, (C₁-C₆)alkyl, or (C₁-C₆)alkoxy, wherein said alkyl is optionally substituted with one to nine halo; R¹³ is hydrogen or (C₁-C₆)alkyl wherein said alkyl is optionally substituted with one to nine halo; and R¹⁴ is hydrogen, aryl, (C₁-C₆)alkyl, or (C₁-C₆)alkoxy wherein said alkyl is optionally substituted with one to nine halo;
each R⁰ is independently hydrogen, halo, (C₁-C₆)alkyl, hydroxy, (C₁-C₆)alkoxy, amino, amido, cyano, oxo, carboxamoyl, carboxy, or (C₁-C₆)alkyloxycarbonyl, wherein said alkyl or alkoxy substituent is optionally independently substituted with one or two oxo, one or two hydroxy, or one to nine halo; and
R¹, R², R³, R⁴, R⁵, R⁶, and R⁷ are each independently hydrogen, halo, cyano, hydroxy, (C₁-C₆)alkyl, (C₁-C₆)alkoxy, or (C₁-C₆)alkylthio wherein said alkyl, alkoxy, and alkylthio substituents are each optionally substituted independently with one to nine halo, one or two cyano or one or two hydroxy.

2. A compound of Formula I 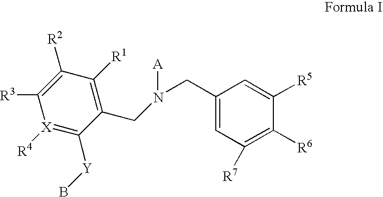
a prodrug thereof, or a pharmaceutically acceptable salt of said compound or of said prodrug; wherein
A is —COO(C₁-C₄)alkyl, cyano, —CHO, —CONH₂, —CO(C₁-C₄)alkyl, triazolyl, tetrazolyl, oxadiazolyl, isoxazolyl, pyrazolyl, or thiadiazolyl and A is mono-, di- or tri-substituted with R⁰;
X is C or N, wherein H X is N, R⁴ is absent;
Y is a bond, —O—, —CR¹¹R¹²—, —CR¹¹R¹²—O—, or —O—CR¹¹R¹²—, wherein R¹¹ and R¹² are each independently hydrogen or (C₁-C₆)alkyl wherein said (C₁-C₆)alkyl is optionally substituted with one to nine halo, or R¹¹ and R¹² may be taken together to form a (C₃-C₆)cycloalkyl optionally substituted with one to nine halo;
B is aryl or heteroaryl wherein B is optionally mono-, di- or tri-substituted independently with (C₀-C₆)alkyl-NR⁸R⁹, (C₀-C₆)alkyl-CO—NR⁸R⁹, (C₀-C₆)alkyl-CO—OR¹⁰), (C₀-C₆)alkyl-NR¹³—CO—O—R¹⁰, (C₁-C₆)alkyl-O—CO—NR⁸R⁹, O—(C₁-C₆)alkyl-CO—O—R¹⁰, (C₀-C₆)alkyl-aryl, (C₀-C₆)alkyl-heteroaryl, O—(C₀-C₆)alkyl-aryl, O—(C₀-C₆)alkyl-heteroaryl, (C₀-C₆)alkyl-O-aryl, (C₀-C₆)alkyl-O-heteroaryl, halo, (C₂-C₆)alkenyl, (C₁-C₆)alkyl, hydroxy, (C₁-C₆)alkoxy, (C₁-C₄)alkylthio, nitro, cyano, oxo, (C₁-C₆)alkylcarbonyl, or (C₁-C₆)alkyloxycarbonyl wherein said (C₁-C₆)alkyl and (C₁-C₆)alkoxy substituents are each optionally substituted independently with one to nine halo, one or two hydroxy, one or two (C₁-C₆)alkoxy, one or two amino, one or two nitro, cyano, oxo, or carboxy, wherein R⁸ and R⁹ are each independently hydrogen, (C₁-C₆)alkyl, (C₁-C₆)alkoxy, or carboxy, R¹⁰ is hydrogen, (C₁-C₆)alkyl, or (C₁-C₆)alkoxy, and R¹³ is hydrogen or (C₁-C₆)alkyl wherein said (C₁-C₆)alkyl is optionally substituted with one to nine halo;
each R⁰ is independently hydrogen, halo, (C₁-C₆)alkyl, hydroxy, (C₁-C₆)alkoxy, amino, amido, cyano, oxo, carboxamoyl, carboxy, or (C₁-C₆)alkyloxycarbonyl, wherein said (C₁-C₆)alkyl substituent is optionally independently substituted with one or two oxo, one or two hydroxy, or one to nine halo; and
R¹, R², R³, R⁴, R⁵, R⁶, and R⁷ are each independently hydrogen, halo, cyano, hydroxy, (C₁-C₆)alkyl, (C₁-C₆)alkoxy, or (C₁-C₆)alkylthio wherein said (C₁-C₆)alkyl, (C₁-C₆)alkoxy, and (C₁-C₆)alkylthio substituents are each optionally substituted independently with one to nine halo, one or two cyano or one or two hydroxy.

3. A compound according to claim 1 wherein Y is a bond.

4. A compound according to claim 2 wherein Y is a bond.

5. A compound according to claim 3 or 4, wherein R¹ and R⁶ are each hydrogen; R⁴ is absent or is hydrogen; and R², R³, R⁵, and R⁷ are each independently hydrogen, cyano, (C₁-C₆)alkyl or (C₁-C₆)alkoxy wherein said alkyl and alkoxy substituents each are optionally substituted independently with one to nine fluorines.

6. A compound according to claim 5, wherein X is C; and R², R³, R⁵, and R⁷ are each hydrogen, methyl, cyano, or CF₃.

7. A compound according to claim 1 or 3 wherein X is C; R¹, R⁴ and R⁶ are each hydrogen; R², R³, R⁵, and R⁷ are each hydrogen, methyl, cyano, or CF₃; and A is —COOCH₂CH₃, —COOCH₃, cyano, —CHO, —CONH₂, —COCH₂CH₃, —COCH₃, 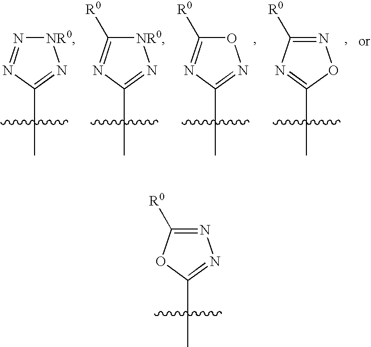
wherein each R⁰ is independently hydrogen, (C₁-C₃)alkyl, or (C₁-C₃)alkoxy, wherein the alkyl or alkoxy is optionally independently substituted with one to nine halo or hydroxyl.

8. A compound according to claim 7, wherein B is phenyl or pyridyl optionally mono- or di-substituted independently with —(C₀-C₆)alkyl-NR⁸R⁹, —(C₀-C₆)alkyl-CO—OR¹⁰, —(C₀-C₆)alkyl-NR¹³—(C₀-C₆)alkyl-CO—O—R¹⁰, —(C₁-C₆)alkyl-O—CO—NR⁸R⁹, —O—(C₁-C₆)alkyl-CO—O—R¹⁰, —(C₀-C₆)alkyl-1-tetrazolyl, halo, (C₁-C₆)alkyl, —(C₀-C₆)alkyl-heterocycle, (C₁-C₆)alkoxy, cyano, —CO—(C₁-C₆)alkyl, or —CO—O—(C₁-C₆)alkyl, wherein said alkyl and alkoxy substituents each optionally substituted independently with one to four fluorines or one or two hydroxy.

9. A compound according to claim 7, wherein each R⁰ is independently hydrogen, CH₃ or CF₃; and
B is 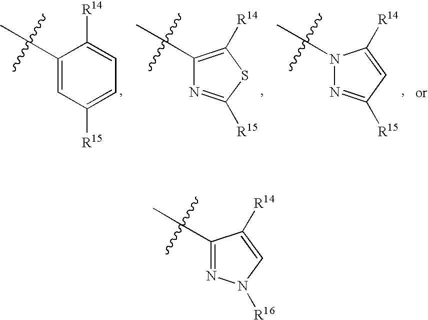
wherein R¹⁴ is halo, cyano, (C₁-C₆)alkyl or —O—(C₁-C₆)alkyl wherein said alkyl substituent is optionally substituted with one to four fluorines; R¹⁵ is —(C₀-C₆)alkyl-NR⁸R⁹, —(C₀-C₆)alkyl-CO—OR¹⁰, —(C₀-C₆)alkyl-NR¹³—(C₀-C₆)alkyl-CO—O—R¹⁰, —(C₁-C₆)alkyl-O—CO—NR⁸R⁹, —O—(C₁-C₆)alkyl-CO—O—R¹⁰, —(C₀-C₆)alkyl-heterocycle, —(C₀-C₆)alkyl-1-tetrazolyl, halo, (C₁-C₆)alkyl, (C₁-C₆)alkoxy, cyano, —CO—(C₁-C₆)alkyl, or —CO—O—(C₀-C₆)alkyl, wherein said alkyl and alkoxy substituents are each optionally substituted independently with one to four fluorines or one or two hydroxyl; and R¹⁶ is —(C₀-C₆)alkyl-CO—OR¹⁰, —(C₂-C₆)alkyl-NR¹³—CO—O—R¹⁰, —(C₂-C₆)alkyl-O—CO—NR⁸R⁹, —(C₀-C₆)alkyl-1-tetrazolyl, (C₁-C₆)alkyl, or —CO—(C₁-C₆)alkyl, wherein said alkyl substituent is optionally substituted with one to four fluorines or one or two hydroxyl.

10. A compound selected from the group consisting of:
N-[3,5-bis(trifluoromethyl)benzyl]-N-{[5′-isopropyl-2′-methoxy-4-(trifluoromethyl)biphenyl-2-yl]methyl}-2-methyl-2H-tetrazol-5-amine;
N-[3,5-bis(trifluoromethyl)benzyl]-N-{[2′-methoxy-5′-methyl(trifluoromethyl)biphenyl-2-yl]methyl}-2-methyl-2H-tetrazol-5-amine;
Methyl-[3,5-bis(trifluoromethyl)benzyl]{[5′-isopropyl-2′-methoxy-4-(trifluoromethyl)biphenyl-2-yl]methyl}carbamate;
2′-{[[3,5-bis(trifluoromethyl)benzyl](2-methyl-2H-tetrazol-5-yl)amino]methyl}-6-methoxy-4′-(trifluoromethyl)biphenyl-3-carbaldehyde;
N-[3,5-bis(trifluoromethyl)benzyl]-N-{[2′-chloro-5′-methyl-4-(trifluoromethyl)biphenyl-2-yl]methyl}-2-methyl-2H-tetrazol-5-amine;
[2′-{[[3,5-bis(trifluoromethyl)benzyl](2-methyl-2H-tetrazol-5-yl)amino]methyl}-6-methoxy-4′-(trifluoromethyl)biphenyl-3-yl]acetonitrile;
N-[3,5-bis(trifluoromethyl)benzyl]-N-{[2′,5′-dimethoxy-4-(trifluoromethyl)biphenyl-2-yl]methyl}-2-methyl-2H-tetrazol-5-amine;
2′-{[[3,5-bis(trifluoromethyl)benzyl](2-methyl-2H-tetrazol-5-yl)amino]methyl}-6-methoxy-4′-(trifluoromethyl)biphenyl-3-carbonitrile;
N-[3,5-bis(trifluoromethyl)benzyl]-N-{[5′-isopropyl-2′-methoxy-4-(trifluoromethyl)biphenyl-2-yl]methyl}acetamide;
N-[3,5-bis(trifluoromethyl)benzyl]-N-{[5′-fluoro-2′-methoxy-4-(trifluoromethyl)biphenyl-2-yl]methyl}-2-methyl-2H-tetrazol-5-amine;
N-[3,5-bis(trifluoromethyl)benzyl]-N-{[3′-isopropyl-4-(trifluoromethyl)biphenyl-2-yl]methyl}-2-methyl-2H-tetrazol-5-amine;
N-[3,5-bis(trifluoromethyl)benzyl]-N-{[2′-methoxy-4-(trifluoromethyl)biphenyl-2-yl]methyl}-2-methyl-2H-tetrazol-5-amine;
N-[3,5-bis(trifluoromethyl)benzyl]-N-{[2′-(methylthio)-4(trifluoromethyl)biphenyl-2-yl]methyl}-2-methyl-2H-tetrazol-5-amine;
N-[3,5-bis(trifluoromethyl)benzyl]-N-{[2′-(trifluoromethoxy)-4-(trifluoromethyl)biphenyl-2-yl]methyl}-2-methyl-2H-tetrazol-5-amine;
N-[3,5-bis(trifluoromethyl)benzyl]-N-{[2′-fluoro-5′-methyl-4-(trifluoromethyl)biphenyl-2-yl]methyl}-2-methyl-2H-tetrazol-5-amine;
N-[3,5-bis(trifluoromethyl)benzyl]-N-{[2′-methoxy-5′-[(4-methylpiperazin-1-yl)methyl]-4-(trifluoromethyl)biphenyl-2-yl]methyl}-2-methyl-2H-tetrazol-5-amine;
N-[3,5-bis(trifluoromethyl)benzyl]-N-[(5′-isopropyl-2′-methoxybiphenyl-2-yl)methyl]-2-methyl-2H-tetrazol-5-amine;
N-[3,5-bis(trifluoromethyl)benzyl]-N-{[5′-[(dimethylamino)methyl]-2′-methoxy-4-(trifluoromethyl)biphenyl-2-yl]methyl}-2-methyl-2H-tetrazol-5-amine;
Methyl-N-{[2′-{[[3,5-bis(trifluoromethyl)benzyl](2-methyl-2H-tetrazol-5-yl)amino]methyl}-6-methoxy-4′-(trifluoromethyl)biphenyl-3-yl]methyl}-N-methylglycinate;
N-[3,5-bis(trifluoromethyl)benzyl]-N-{[2′-ethoxy(trifluoromethyl)biphenyl-2-yl]methyl}-2-methyl-2H-tetrazol-5-amine;
1-[2′-{[[3,5-bis(trifluoromethyl)benzyl](2-methyl-2H-tetrazol-5-yl)amino]methyl}-6-fluoro-4′-(trifluoromethyl)biphenyl-3-yl]ethanone; and
4-{1-[2-{[[3,5-bis(trifluoromethyl)benzyl](2-methyl-2H-tetrazol-5-yl)amino]methyl}-4-(trifluoromethyl)phenyl]propoxy}benzamide;
or a pharmaceutically acceptable salt of said compound.

11. A method for treating atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal by administering to a mammal in need of such treatment an atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction treating amount of a compound of claim 1, 2 or 10 or a pharmaceutically acceptable salt of said compound.

12. A pharmaceutical composition which comprises a therapeutically effective amount of a compound of claim 1, 2 or 10, or a pharmaceutically acceptable salt of said compound and a pharmaceutically acceptable vehicle, diluent or carrier.

13. A pharmaceutical combination composition comprising: a therapeutically effective amount of a composition comprising
a first compound, said first compound being a compound of claim 1, 2 or 10, or a pharmaceutically acceptable salt of said compound;
a second compound, said second compound being an HMG CoA reductase inhibitor, an MTP/Apo B secretion inhibitor, a PPAR modulator, a bile acid reuptake inhibitor, a cholesterol absorption inhibitor, a cholesterol synthesis inhibitor, a fibrate, niacin, slow-release niacin, a combination of niacin and lovastatin, a combination of niacin and simvastatin, a combination of niacin and atorvastatin, a combination of amlodipine and atorvastatin, an ion-exchange resin, an antioxidant, an ACAT inhibitor or a bile acid sequestrant; and
a pharmaceutical vehicle, diluent or carrier.

14. A pharmaceutical combination composition according to claim 13 wherein the second compound is an HMG-CoA reductase inhibitor, a PPAR modulator, or niacin.

15. A pharmaceutical combination composition according to claim 14 wherein the second compound is fenofibrate, gemfibrozil, lovastatin, simvastatin, pravastatin, fluvastatin, atorvastatin, rivastatin, rosuvastatin or pitavastatin.
